SEQUENCE LISTING (22 sequences)

SEQ ID NO: 1 - protein, 10 aa, Mus musculus
GYTFTSYWMQ

SEQ ID NO: 2 - protein, 17 aa, Mus musculus
TIYPGDGDTSYAQKFKG

SEQ ID NO: 3 - protein, 9 aa, Mus musculus
WGYAYDIDN

SEQ ID NO: 4 - protein, 16 aa, Mus musculus
KSSQSLLDSDGKTYLN

SEQ ID NO: 5 - protein, 7 aa, Mus musculus
LVSKLDS

SEQ ID NO: 6 - protein, 9 aa, Mus musculus
WQGTHFPYT

SEQ ID NO: 7 - protein, 109 aa, Artificial sequence
  humanized variable heavy chain
EVQLVESGGGLVQPGGSLRLSCAASGYTFTSYWMQWVRQAPGKGLEWIGTIYPGDGDTSYAQKFKGRATLSTDKSKNTAYLQMNSLRAEDTAVYYCARWGYAYDIDNWG

SEQ ID NO: 8 - protein, 112 aa, Artificial sequence
  humanized variable light chain
DIQMTQSPSSLSASVGDRVTITCKSSQSLLDSDGKTYLNWLQQKPGKAPKRLIYLVSKLDSGVPSRFSGSGSGTDFTLTISSLQPEDFATYYCWQGTHFPYTFGQGTKVEIK

SEQ ID NO: 9 - protein, 109 aa, Artificial sequence
  humanized variable heavy chain.
EVQLVQSGAEVKKPGASVKVSCKASGYTFTSYWMQWVRQAPGQGLEWIGTIYPGDGDTSYAQKFKGRVTITRDTSTSTAYLELSSLRSEDTAVYYCARWGYAYDIDNWG

SEQ ID NO: 10 - protein, 442 aa, Homo sapiens
MQPPPSLCGRALVALVLACGLSRIWGEERGFPPDRATPLLQTAEIMTPPTKTLWPKGSNASLARSLAPAEVPKGDRTAGSPPRTISPPPCQGPIEIKETFKYINTVVSCLVFVLGIIGNSTLLRIIYKNKCMRNGPNILIASLALGDLLHIVIDIPINVYKLLAEDWPFGAEMCKLVPFIQKASVGITVLSLCALSIDRYRAVASWSRIKGIGVPKWTAVEIVLIWVVSVVLAVPEAIGFDIITMDYKGSYLRICLLHPVQKTAFMQFYKTAKDWWLFSFYFCLPLAITAFFYTLMTCEMLRKKSGMQIALNDHLKQRREVAKTVFCLVLVFALCWLPLHLSRILKLTLYNQNDPNRCELLSFLLVLDYIGINMASLNSCINPIALYLVSKRFKNCFKSCLCCWCQSFEEKQSLEEKQSCLKFKANDHGYDNFRSSNKYSSS

SEQ ID NO: 11 - DNA, 19 bp, Homo sapiens
agcggattctcatggaaca

SEQ ID NO: 12 - DNA, 18 bp, Homo sapiens
ctggtcagccaggagctt

SEQ ID NO: 13 - DNA, 24 bp, Homo sapiens
tccacaagctgaaggcagacaagg

SEQ ID NO: 14 - DNA, 22 bp, Homo sapiens
tcactgaattcctgcattaacc

SEQ ID NO: 15 - DNA, 24 bp, Homo sapiens
gcataagcatgacttaaagcagtt

SEQ ID NO: 16 - DNA, 33 bp, Homo sapiens
aattgctctgtatttggtgagcaaaagattcaa

SEQ ID NO: 17 - DNA, 24 bp, Homo sapiens
cacatcaaagacagcatctaagaa

SEQ ID NO: 18 - DNA, 25 bp, Homo sapiens
caagttggaaaatacagtcaacatt

SEQ ID NO: 19 - DNA, 37 bp, Homo sapiens
ttttgttctgtcctggaattattttagtagtgtttca

SEQ ID NO: 20 - DNA, 20 bp, Homo sapiens
gaagatggtgatgggatttc

SEQ ID NO: 21 - DNA, 19 bp, Homo sapiens
gaaggtgaaggtcggagtc

SEQ ID NO: 22 - DNA, 20 bp, Homo sapiens
caagcttcccgttctcagcc

(19) United States
(12) Patent Application Publication
(10) Pub. No.: US 2014/0341916 A1
(43) Pub. Date: Nov. 20, 2014

(54) THERAPEUTIC COMBINATIONS AND METHODS OF TREATING MELANOMA

(71) Applicant: Genentech, Inc., South San Francisco, CA (US)

(72) Inventors: Paul Polakis, Mill Valley, CA (US); Jyoti Asundi, Foster City, CA (US); Suzanna Clark, Pacifica, CA (US)

(73) Assignee: GENENTECH, INC., South San Francisco, CA (CA)

(21) Appl. No.: 14/354,362

(22) PCT Filed: Oct. 24, 2012
(86) PCT No.: PCT/US2012/061533

§ 371 Date: Apr. 25, 2014

Related U.S. Application Data
(60) Provisional application No. 61/552,893, filed on Oct. 28, 2011.
(60) Provisional application No. 61/678,987, filed on Aug. 2, 2012.

(51) Int. Cl. C07K 16/28; A61K 39/395; C07K 16/30; A61K 31/437; A61K 31/4523

(52) U.S. Cl. CPC C07K 16/2869; A61K 31/437; A61K 31/4523; C07K 16/3053; A61K 39/39558
USPC 424/139.1; 424/178.1

(57) ABSTRACT

The invention provides therapeutic combinations of anti-ETBR antibodies and MAP kinase inhibitors and methods of using the same to treat melanoma.

### RELATED APPLICATIONS

This application claims the benefit under 35 USC 119(e) of U.S. Provisional Application No. 61/552,893 filed 28 Oct. 2011 and U.S. Provisional Application No. 61/678,978 filed 2 Aug. 2012, the contents of which are incorporated herein by reference.

### FIELD OF THE INVENTION

This invention concerns in general, a treatment of melanoma by using certain antibody and small molecule drug combinations.

### BACKGROUND

Melanoma is an aggressive form of skin cancer that has recently undergone an alarming increase in incidence (Thompson J F et al., Cutaneous melanoma in the era of molecular profiling. Lancet 2009; 374:362-5). Although cures can be achieved with surgical resection of localized lesions, the advanced stages of melanoma are only poorly responsive to currently approved therapies. The 5-year survival rate for stage IV metastatic melanoma is approximately 10% (Thompson, Lancet 2009). New therapeutic approaches, including antisense to Bcl2, antibodies to CTLA4, small molecule RAF kinase inhibitors, and adoptive immunotherapy, are currently in clinical testing for metastatic melanoma (Ascierto P A et al., Melanoma: a model for testing new agents in combination therapies. J Transl Med 2010; 8:38-45). The results from some of these recent studies seem to be encouraging, but a durable impact on overall survival may require therapeutic combinations including additional new agents.

However, it is recognized that melanomas can demonstrate molecular variations, for example in certain signal transduction pathways necessary for cell responsiveness to growth factors. Therefore, rather than treating melanoma as a single disease, at have been made to stratify it into molecular subtypes in order to treat each subtype with the most appropriate therapies.

One subtype of melanoma harbors aberrations in the MAP kinase pathway. The MAPK pathway is a phosphorylation-driven signal transduction cascade that couples intracellular responses to the binding of growth factors to cell surface receptors. This pathway regulates several processes including cell proliferation and differentiation, and is often dysregulated in a variety of cancers. (Sebolt-Leopold J S, Herrera R. Targeting the mitogen-activated protein kinase cascade to treat cancer. Nat Rev Cancer. 2004; 4:937-947). The classical MAPK pathway consists of RAS, RAF, MEK and ERK, where RAS triggers the formation of a RAF/MEK/ERK kinase complex which then drives transcription of key regulators through protein phosphorylation. The inhibition of MAPK signaling with agents targeted toward critical proteins in the pathway has the potential to inhibit growth in a variety of tumor types (Wong K-K et al., Recent developments in anti-cancer agents targeting the Ras/Raf/MEK/ERK pathway. Recent Pat Anticancer Drug Discov. 2009; 4:28-35.

Inappropriate activation of the MEK/ERK pathway promotes cell growth in the absence of exogenous growth factors. GDC-0973 (a.k.a. XL518) is a potent and highly selective small molecule inhibitor of MEK1/2, a MAPK kinase that activates ERK1/2 (Johnston S. XL518, a potent, selective, orally bioavailable MEK1 inhibitor, downregulates the Ras/Raf/MEK/ERK pathway in vivo, resulting in tumor growth inhibition and regression in preclinical models. Presented at: AACR-NCI-EORTC Symposium on Molecular Targets and Cancer Therapeutics; Oct. 22, 2007; San Francisco, Calif. Abstract C209). As a consequence, the oncogenic signal from cell surface, Ras and Raf, to ERK is interrupted. Sustained inhibition of ERK activation translates into decreased proliferation and induction of apoptosis. In multiple preclinical studies, GDC-0973 has been shown to inhibit cell growth and induce tumor regression.

Recently, vemurafenib, also known as Zelboraf®, which is a B-Raf enzyme inhibitor, was approved by the U.S. Food and Drug Administration for the treatment of late-stage melanoma. Vemurafenib has been shown to cause programmed cell death in melanoma cell lines (Sala E, et al., BRAF silencing by short hairpin RNA or chemical blockade by PLX4032 leads to different responses in melanoma and thyroid carcinoma cells. Mol. Cancer Res. 6 (5): 751-9 (May 2008). Vemurafenib interrupts the B-Raf/MEK step on the B-Raf/MEK/ERK pathway if the B-Raf has the common V600E mutation. Vemurafenib is effective in melanoma patients whose cancer has a V600E BRAF mutation (that is, at amino acid position number 600 on the B-RAF protein, the normal valine is replaced by glutamic acid). About 60% of melanomas have the V600E BRAF mutation. Melanoma cells without this mutation do not appear to be inhibited by vemurafenib; it paradoxically stimulates normal BRAF and may promote tumor growth (Hatzivassiliou G et al., RAF inhibitors prime wild-type RAF to activate the MAPK pathway and enhance growth. Nature 464 (7287): 431-5; Halaban R et al., PLX4032, a Selective BRAF (V600E) Kinase Inhibitor, Activates the ERK Pathway and Enhances Cell Migration and Proliferation of BRAF(WT) Melanoma Cells. Pigment Cell Melanoma Res 23 (2): 190-200 (February 2010). While clinical trials revealed an improved survival, improved objective response rate, and improved progression-free survival for those patients treated with vemurafenib as compared to DTIC, disease recurrence is likely (Nazarian R. et al., Melanomas acquire resistance to B-RAF(V600E) inhibition by RTK or N-RAS upregulation. Nature Vol: 468, Pages: 973-977 (16 Dec. 2010)).

Despite advances in melanoma cancer therapy, there is a great need for additional therapeutic treatments capable of effectively inhibiting neoplastic cell growth. Accordingly, it is an objective of the present invention to identify combinations of therapeutic agents to produce compositions of matter useful in the therapeutic treatment of melanoma cancers.

### SUMMARY

The present invention contemplates a method of tumor growth inhibition (TGI) in a subject suffering from melanoma comprising administering to the subject an effective amount of an anti-endothelin B receptor (ETBR) antibody drug conjugate in combination with an effective amount of a MAP kinase inhibitor.

In one aspect, the combination of an anti-ETBR antibody drug conjugate and a MAP kinase inhibitor is synergistic. In another aspect, with respect to the synergistic combination, the TGI is greater than the TGI seen using an anti-ETBR antibody drug conjugate alone or greater than the TGI seen using a MAP kinase inhibitor alone. In yet a further aspect, with respect to the synergistic combination, the TGI is about 10% greater, or about 15% greater, or about 20% greater, or about 25% greater, or about 30% greater, or about 35% greater, or about 40% greater, or about 45% greater, or about 50% greater, or about 55% greater, or about 60% greater, or about 65% greater, or about 70% greater than use of an anti-ETBR antibody drug conjugate alone or the TGI is about 10% greater, or about 15% greater, or about 20% greater, or about 25% greater, or about 30% greater, or about 35% greater, or about 40% greater, or about 45% greater, or about 50% greater, or about 55% greater, or about 60% greater, or about 65% greater, or about 70% greater than use of a MAP kinase inhibitor alone.

In another aspect of the invention described above, the anti-ETBR antibody specifically binds an ETBR epitope consisting of amino acids number 64 to 101 of SEQ ID NO:10. In yet another aspect of the claimed method, the anti-ETBR antibody has three variable heavy chain CDRs and three variable light chain CDRs wherein VH CDR1 is SEQ ID NO:1, VH CDR2 is SEQ ID NO:2, VH CDR3 is SEQ ID NO:3 and wherein VL CDR1 is SEQ ID NO:4, VL CDR2 is SEQ ID NO:5, VL CDR3 is SEQ ID NO:6. In still another aspect of the claimed method, the anti-ETBR antibody has a variable heavy chain and a variable light chain, wherein said VH is SEQ ID NO:7 or 9. A further aspect of this method also includes an anti-ETBR antibody also having a VL which is SEQ ID NO:8.

In one aspect of the claimed method described above, the anti-ETBR antibody is conjugated to a cytotoxin, wherein said cytotoxin is a cytotoxic agent that is selected from the group consisting of toxins, antibiotics, radioactive isotopes and nucleolytic enzymes, and wherein said cytotoxin is a toxin. In another aspect of the claimed invention, the toxin is selected from the group consisting of maytansinoid, calicheamicin and auristatin. In yet another aspect of the invention, the toxin is a maytansinoid.

In one aspect of the claimed method described above, the MAP kinase inhibitor is a BRAF inhibitor. In yet another aspect of the claimed method described above, the BRAF inhibitor is propane-1-sulfonic acid {3-[5-(4-chlorophenyl)-1H-pyrrolo[2,3-b]pyridine-3-carbonyl]-2,4-difluoro-phenyl}-amide. In another aspect, the BRAF inhibitor has the following chemical structure:

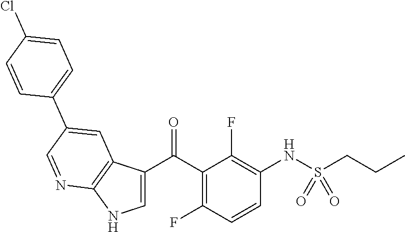 [SMILES: CCCS(=O)(=O)Nc1ccc(F)c(C(=O)c2c[nH]c3ncc(-c4ccc(Cl)cc4)cc23)c1F]

In another aspect of the claimed method described above, the MAP kinase inhibitor is a MEK inhibitor. In yet another aspect of the claimed method described above, the MEK inhibitor is (S)-(3,4-difluoro-2-((2fluoro-4-iodophenyl)amino)phenyl)(3-hydroxy-3-(piperidin-2yl)azetidin-1-yl)methanone. In still another aspect of the claimed method described above, the MEK inhibitor has the following chemical structure:

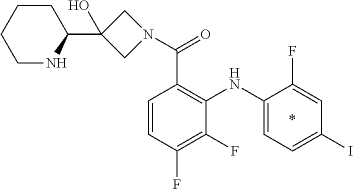 [SMILES: O=C(c1ccc(F)c(F)c1Nc1ccc(I)cc1F)N1CC(O)([C@@H]2CCCCN2)C1]

In one aspect of the invention, it is contemplated that a method of treating melanoma comprising administering to a subject in need thereof a therapeutically effective amount of a MAP kinase inhibitor and an anti-ETBR antibody is described. In another aspect of the claimed method described above, said melanoma is ETBR positive. In yet another aspect of the claimed method, said melanoma is metastatic. In still a further aspect of the claimed method, said subject has not had prior therapy with a MAP kinase inhibitor. In yet a further aspect of the claimed method, said subject has a V600E BRAF gene mutation or said subject is BRAF wildtype, having no V600E BRAF mutation. In still a further aspect of the claimed method, the subject has not had prior therapy with a MAP kinase inhibitor.

In another aspect of the claimed method described above, the combination of an anti-ETBR antibody and a MAP kinase inhibitor is synergistic. In another aspect, with respect to the synergistic combination, the TGI is greater than the TGI seen using an anti-ETBR antibody alone or greater than the TGI seen using a MAP kinase inhibitor alone. In yet a further aspect, with respect to the synergistic combination, the TGI is about 10% greater, or about 15% greater, or about 20% greater, or about 25% greater, or about 30% greater, or about 35% greater, or about 40% greater, or about 45% greater, or about 50% greater, or about 55% greater, or about 60% greater, or about 65% greater, or about 70% greater than use of an anti-ETBR antibody alone or the TGI is about 10% greater, or about 15% greater, or about 20% greater, or about 25% greater, or about 30% greater, or about 35% greater, or about 40% greater, or about 45% greater, or about 50% greater, or about 55% greater, or about 60% greater, or about 65% greater, or about 70% greater than use of a MAP kinase inhibitor alone.

In another aspect of the invention described above, the anti-ETBR antibody specifically binds an ETBR epitope consisting of amino acids number 64 to 101 of SEQ ID NO:10. In yet another aspect of the claimed method, the anti-ETBR antibody has three variable heavy chain CDRs and three variable light chain CDRs wherein VH CDR1 is SEQ ID NO:1, VH CDR2 is SEQ ID NO:2, VH CDR3 is SEQ ID NO:3 and wherein VL CDR1 is SEQ ID NO:4, VL CDR2 is SEQ ID NO:5, VL CDR3 is SEQ ID NO:6. In still another aspect of the claimed method, the anti-ETBR antibody has a variable heavy chain and a variable light chain, wherein said VH is SEQ ID NO:7 or 9. A further aspect of this method also includes an anti-ETBR antibody also having a VL which is SEQ ID NO:8.

In one aspect of the claimed method described above, the anti-ETBR antibody is conjugated to a cytotoxin, wherein said cytotoxin is cytotoxic agent is selected from the group consisting of toxins, antibiotics, radioactive isotopes and nucleolytic enzymes, and wherein said cytotoxin is a toxin. In another aspect of the claimed invention, the toxin is selected from the group consisting of maytansinoid, calicheamicin and auristatin. In yet another aspect of the invention, the toxin is a maytansinoid.

In one aspect of the claimed method described above, the MAP kinase inhibitor is a BRAF inhibitor. In yet another aspect of the method described above, the BRAF inhibitor is propane-1-sulfonic acid {3-[5-(4-chlorophenyl)-1H-pyrrolo[2,3-b]pyridine-3-carbonyl]-2,4-difluoro-phenyl}-amide. In another aspect, the BRAF inhibitor has the following chemical structure:

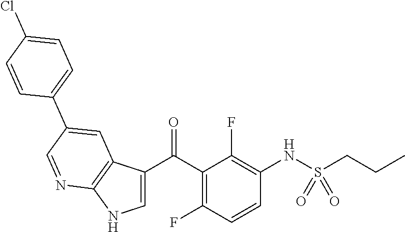 [SMILES: CCCS(=O)(=O)Nc1ccc(F)c(C(=O)c2c[nH]c3ncc(-c4ccc(Cl)cc4)cc23)c1F]

In another aspect of the claimed method described above, the MAP kinase inhibitor is a MEK inhibitor. In yet another aspect of the claimed method described above, the MEK inhibitor is (S)-(3,4-difluoro-2-((2fluoro-4-iodophenyl)amino)phenyl)(3-hydroxy-3-(piperidin-2yl) azetidin-1-yl)methanone. In still another aspect of the claimed method described above, the MEK inhibitor has the following chemical structure:

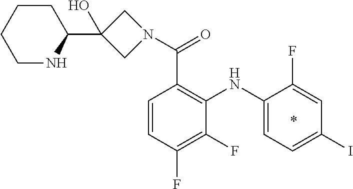 [SMILES: O=C(c1ccc(F)c(F)c1Nc1ccc(I)cc1F)N1CC(O)([C@@H]2CCCCN2)C1]

In one aspect of the invention, it is contemplated that a method of treating melanoma comprising administering to a subject in need thereof a therapeutically effective amount of a MAP kinase inhibitor and an anti-ETBR antibody drug conjugate is described, wherein the MAP kinase inhibitor is administered first to said subject in need thereof. In another aspect of the invention, said anti-ETBR antibody drug conjugate is administered after administration of said MAP kinase inhibitor. Alternatively, contemplated methods of the invention include where the anti-ETBR antibody and the MAP kinase inhibitor are administered simultaneously.

In one aspect of the invention, it is contemplated that a method of treating melanoma comprising administering to a subject in need thereof a therapeutically effective amount of a MAP kinase inhibitor and an anti-ETBR antibody drug conjugate is described, wherein the anti-ETBR antibody drug conjugate and the MAP kinase inhibitor are administered sequentially, wherein the anti-ETBR antibody drug conjugate is administered to the subject first and the MAP kinase inhibitor is administered to the subject after administration of the anti-ETBR antibody drug conjugate.

In one aspect of the invention, it is contemplated that a method of treating melanoma comprising administering to a subject in need thereof a therapeutically effective amount of a MAP kinase inhibitor and an anti-ETBR antibody drug conjugate is described, wherein the MAP kinase inhibitor is administered to the subject first and the anti-ETBR antibody drug conjugate is administered to the subject after administration of the MAP kinase inhibitor.

In furtherance of the above contemplated aspects of the invention, it is further contemplated that a method of treating melanoma comprising administering to a subject in need thereof a therapeutically effective amount of a MAP kinase inhibitor and an anti-ETBR antibody drug conjugate is described, wherein said anti-ETBR antibody drug conjugate is administered intraveneously. It is also contemplated that the anti-ETBR antibody drug conjugate is dosed at about 0.1 mpk, or about 0.2 mpk, or about 0.3 mpk, or about 0.5 mpk, or about 1 mpk, or about 5 mpk, or about 10 mpk, or about 15 mpk, or about 20 mpk, or about 25 mpk, or about 30 mpk in the claimed methods of the invention.

In furtherance of the above contemplated aspects of the invention, it is further contemplated that a method of treating melanoma comprising administering to a subject in need thereof a therapeutically effective amount of a MAP kinase inhibitor and an anti-ETBR antibody drug conjugate is described, wherein the MAP kinase inhibitor is administered orally. It is also contemplated that the BRAF inhibitor is dosed at about 1 mpk, or about 2 mpk, or about 3 mpk, or about 4 mpk, or about 5 mpk, or about 6 mpk, or about 7 mpk, or about 8 mpk, or about 9 mpk, or about 10 mpk, or about 11 mpk, or about 12 mpk, or about 15 mpk, or about 20 mpk or about 30 mpk in the claimed methods of the invention.

In one aspect of the invention, it is contemplated that an article of manufacture is used for TGI in a subject suffering from melanoma comprising a package comprising an anti-ETBR antibody drug conjugate composition and a MAP kinase inhibitor composition. It is further contemplated that said anti-ETBR antibody drug conjugate specifically binds an ETBR epitope consisting of amino acids number 64 to 101 of SEQ ID NO:10. Alternatively, it is also contemplated that said anti-ETBR antibody has three variable heavy chain CDRs and three variable light chain CDRs wherein VH CDR1 is SEQ ID NO:1, VH CDR2 is SEQ ID NO:2, VH CDR3 is SEQ ID NO:3 and wherein VL CDR1 is SEQ ID NO:4, VL CDR2 is SEQ ID NO:5, VL CDR3 is SEQ ID NO:6. A further alternative that is contemplated is an anti-ETBR antibody has a variable heavy chain and a variable light chain, wherein said VH is SEQ ID NO:7 or 9. In yet another alternative, an anti-ETBR antibody has a variable heavy chain and a variable light chain, wherein said VH is SEQ ID NO:7 or 9 and the VL is SEQ ID NO:8. In another aspect of the article of manufacture, the anti-ETBR antibody is conjugated to a cytotoxin, wherein said cytotoxin is cytotoxic agent is selected from the group consisting of toxins, antibiotics, radioactive isotopes and nucleolytic enzymes. In one further aspect, the cytotoxin is a toxin wherein said toxin is selected from the group consisting of maytansinoid, calicheamicin and auristatin. In one aspect, the toxin is a maytansinoid.

In one aspect of the method of treating melanoma described above, it is also contemplated that the MAP kinase inhibitor is a BRAF inhibitor. In yet another aspect of the method described above, the BRAF inhibitor is propane-1-sulfonic acid {3-[5-(4-chlorophenyl)-1H-pyrrolo[2,3-b]pyridine-3-carbonyl]-2,4-difluoro-phenyl}-amide. Further, it is contemplated that the BRAF inhibitor has the following chemical structure:

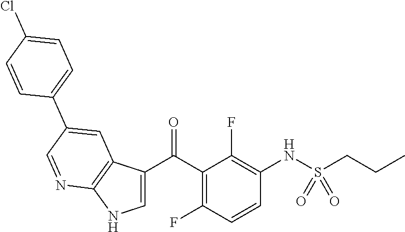 [SMILES: CCCS(=O)(=O)Nc1ccc(F)c(C(=O)c2c[nH]c3ncc(-c4ccc(Cl)cc4)cc23)c1F]

In another aspect of the claimed method described above, the MAP kinase inhibitor is a MEK inhibitor. In yet another aspect of the claimed method described above, the MEK inhibitor is (S)-(3,4-difluoro-2-((2fluoro-4-iodophenyl)amino)phenyl)(3-hydroxy-3-(piperidin-2yl) azetidin-1-yl)methanone. In still another aspect of the claimed method described above, the MEK inhibitor has the following chemical structure:

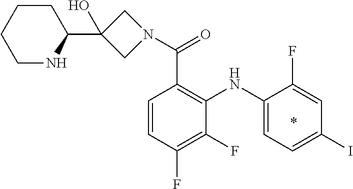 [SMILES: O=C(c1ccc(F)c(F)c1Nc1ccc(I)cc1F)N1CC(O)([C@@H]2CCCCN2)C1]

In one aspect of the invention, it is contemplated that an article of manufacture for treating melanoma in a subject comprising a package comprising an anti-ETBR antibody drug conjugate composition and a MAP kinase inhibitor composition. It is further contemplated that said anti-ETBR antibody specifically binds an ETBR epitope consisting of amino acids number 64 to 101 of SEQ ID NO:10. Alternatively, it is also contemplated that said anti-ETBR antibody has three variable heavy chain CDRs and three variable light chain CDRs wherein VH CDR1 is SEQ ID NO:1, VH CDR2 is SEQ ID NO:2, VH CDR3 is SEQ ID NO:3 and wherein VL CDR1 is SEQ ID NO:4, VL CDR2 is SEQ ID NO:5, VL CDR3 is SEQ ID NO:6. A further alternative that is contemplated is an anti-ETBR antibody has a variable heavy chain and a variable light chain, wherein said VH is SEQ ID NO:7 or 9. In yet another alternative, an anti-ETBR antibody has a variable heavy chain and a variable light chain, wherein said VH is SEQ ID NO:7 or 9 and the VL is SEQ ID NO:8. In another aspect of the article of manufacture, the anti-ETBR antibody is conjugated to a cytotoxin, wherein said cytotoxin is cytotoxic agent is selected from the group consisting of toxins, antibiotics, radioactive isotopes and nucleolytic enzymes. In one further aspect, the cytotoxin is a toxin wherein said toxin is selected from the group consisting of maytansinoid, calicheamicin and auristatin. In one aspect, the toxin is a maytansinoid.

In one aspect of the article of manufacture described above, it is also contemplated that the MAP kinase inhibitor is a BRAF inhibitor. In yet another aspect of the article of manufacture described above, the BRAF inhibitor is propane-1-sulfonic acid {3-[5-(4-chlorophenyl)-1H-pyrrolo[2,3-b]pyridine-3-carbonyl]-2,4-difluoro-phenyl}-amide. Further, it is contemplated that the BRAF inhibitor has the following chemical structure:

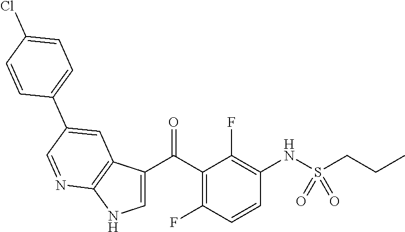 [SMILES: CCCS(=O)(=O)Nc1ccc(F)c(C(=O)c2c[nH]c3ncc(-c4ccc(Cl)cc4)cc23)c1F]

In another aspect of the article of manufacture described above, the MAP kinase inhibitor is a MEK inhibitor. In yet another aspect of the article of manufacture described above, the MEK inhibitor is (S)-(3,4difluoro-2-((2fluoro-4-iodophenyl)amino)phenyl)(3-hydroxy-3-(piperidin-2yl)azetidin-1-yl)methanone. In still another aspect of the article of manufacture described above, the MEK inhibitor has the following chemical structure:

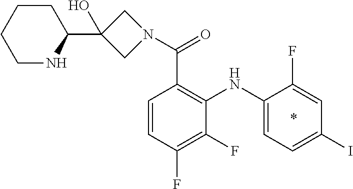 [SMILES: O=C(c1ccc(F)c(F)c1Nc1ccc(I)cc1F)N1CC(O)([C@@H]2CCCCN2)C1]

It is contemplated that one aspect of the invention is use of an anti-ETBR antibody drug conjugate and a MAP kinase inhibitor in the preparation of a medicament for TGI of a melanoma. It is further contemplated that said anti-ETBR antibody specifically binds an ETBR epitope consisting of amino acids number 64 to 101 of SEQ ID NO:10. Alternatively, it is also contemplated that said anti-ETBR antibody has three variable heavy chain CDRs and three variable light chain CDRs wherein VH CDR1 is SEQ ID NO:1, VH CDR2 is SEQ ID NO:2, VH CDR3 is SEQ ID NO:3 and wherein VL CDR1 is SEQ ID NO:4, VL CDR2 is SEQ ID NO:5, VL CDR3 is SEQ ID NO:6. A further alternative that is contemplated is an anti-ETBR antibody has a variable heavy chain and a variable light chain, wherein said VH is SEQ ID NO:7 or 9. In yet another alternative, an anti-ETBR antibody has a variable heavy chain and a variable light chain, wherein said VH is SEQ ID NO:7 or 9 and the VL is SEQ ID NO:8. In another aspect of the article of manufacture, the anti-ETBR antibody is conjugated to a cytotoxin, wherein said cytotoxin is cytotoxic agent is selected from the group consisting of toxins, antibiotics, radioactive isotopes and nucleolytic enzymes. In one further aspect, the cytotoxin is a toxin wherein said toxin is selected from the group consisting of maytansinoid, calicheamicin and auristatin. In one aspect, the toxin is a maytansinoid.

In one aspect of the use of the medicament described above, it is also contemplated that the MAP kinase inhibitor is a BRAF inhibitor. In yet another aspect of the use of the medicament described above, the BRAF inhibitor is propane-1-sulfonic acid {3-[5-(4-chlorophenyl)-1H-pyrrolo[2,3-b]pyridine-3-carbonyl]-2,4-difluoro-phenyl}-amide. Further, it is contemplated that the BRAF inhibitor has the following chemical structure:

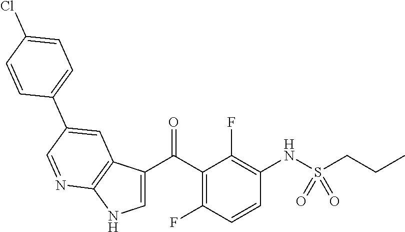 [SMILES: CCCS(=O)(=O)Nc1ccc(F)c(C(=O)c2c[nH]c3ncc(-c4ccc(Cl)cc4)cc23)c1F]

In another aspect of the use of the medicament described above, the MAP kinase inhibitor is a MEK inhibitor. In yet another aspect of the use of the medicament described above, the MEK inhibitor is (S)-(3,4difluoro-2-((2fluoro-4-iodophenyl)amino)phenyl)(3-hydroxy-3-(piperidin-2yl)azetidin-1-yl)methanone. In still another aspect of the use of the medicament described above, the MEK inhibitor has the following chemical structure:

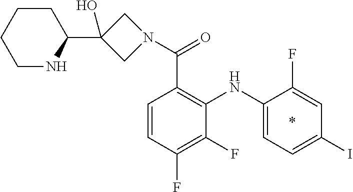 [SMILES: O=C(c1ccc(F)c(F)c1Nc1ccc(I)cc1F)N1CC(O)([C@@H]2CCCCN2)C1]

It is contemplated that one aspect of the invention is use of an article of manufacture comprising an anti-ETBR antibody drug conjugate composition and a MAP kinase inhibitor composition in the preparation of a medicament for TGI of a melanoma. It is further contemplated that said anti-ETBR antibody specifically binds an ETBR epitope consisting of amino acids number 64 to 101 of SEQ ID NO:10. Alternatively, it is also contemplated that said anti-ETBR antibody has three variable heavy chain CDRs and three variable light chain CDRs wherein VH CDR1 is SEQ ID NO:1, VH CDR2 is SEQ ID NO:2, VH CDR3 is SEQ ID NO:3 and wherein VL CDR1 is SEQ ID NO:4, VL CDR2 is SEQ ID NO:5, VL CDR3 is SEQ ID NO:6. A further alternative that is contemplated is an anti-ETBR antibody has a variable heavy chain and a variable light chain, wherein said VH is SEQ ID NO:7 or 9. In yet another alternative, an anti-ETBR antibody has a variable heavy chain and a variable light chain, wherein said VH is SEQ ID NO:7 or 9 and the VL is SEQ ID NO:8. In another aspect of the use of the article of manufacture, the anti-ETBR antibody is conjugated to a cytotoxin, wherein said cytotoxin is cytotoxic agent is selected from the group consisting of toxins, antibiotics, radioactive isotopes and nucleolytic enzymes. In one further aspect, the cytotoxin is a toxin wherein said toxin is selected from the group consisting of maytansinoid, calicheamicin and auristatin. In one aspect, the toxin is a maytansinoid.

In one aspect of the use of the article of manufacture described above, it is also contemplated that the MAP kinase inhibitor is a BRAF inhibitor. In yet another aspect of the use of the article of manufacture described above, the BRAF inhibitor is propane-1-sulfonic acid {3-[5-(4-chlorophenyl)-1H-pyrrolo[2,3-b]pyridine-3-carbonyl]-2,4-difluoro-phenyl}-amide. Further, it is contemplated that the BRAF inhibitor has the following chemical structure:

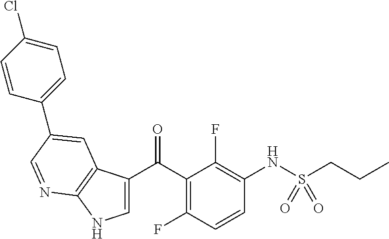 [SMILES: CCCS(=O)(=O)Nc1ccc(F)c(C(=O)c2c[nH]c3ncc(-c4ccc(Cl)cc4)cc23)c1F]

In another aspect of the use of the article of manufacture described above, the MAP kinase inhibitor is a MEK inhibitor. In yet another aspect of the use of the article of manufacture described above, the MEK inhibitor is (S)-(3,4-difluoro-2-((2-fluoro-4-iodophenyl)amino)phenyl)(3-hydroxy-3-(piperidin-2yl)azetidin-1-yl)methanone. In still another aspect of the use of the article of manufacture described above, the MEK inhibitor has the following chemical structure:

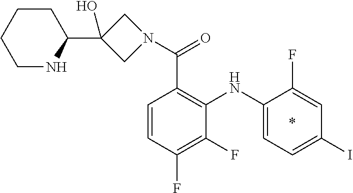 [SMILES: O=C(c1ccc(F)c(F)c1Nc1ccc(I)cc1F)N1CC(O)([C@@H]2CCCCN2)C1]

### BRIEF DESCRIPTION OF THE FIGURES

FIG. 1 is a schematic of the MAP kinase pathway.

FIG. 2 demonstrates the relationship of receptor level to ADC cell killing in vitro. The indicated number of receptor copies/cell was estimated by Scatchard analysis. Panel A shows cell killing by anti-ET_{B}R ADC titration for the melanoma cell line UACC-257X2.2 and panel B for melanoma cell line A2058. The indicated concentrations of anti-ET_{B}R ADC (Hu5E9v1-vc-MMAE), control IgG-vc-MMAE, or equivalent amount of PBS vehicle control were incubated with cells for 5 days and relative cell viability (y-axis) assessed using CellTiter-Glo.

FIG. 3 shows the in vivo efficacy of anti-ETBR ADC in xenografts mouse models. Subcutaneous tumors were established in mice inoculated with UACC-257X2.2 (Panel A) or A2058 (Panel B) cells. When tumor volumes reached approximately 200 mm³ (day 0), animals were given a single IV injection of either control ADC (Control-vc-MMAE) or anti-ETBR ADC (Hu5E9v1-vc-MMAE) at the indicated doses. Average tumor volumes with standard deviations were determined from 10 animals per groups (indicated on graph).

FIG. 4 shows ET_{B}R expression in UACC-257X2.2 melanoma cells treated for 24 h with varying concentrations of BRAFi-945. Panel A shows ETBR transcript normalized to RPL19 transcript. Panel B shows the expression of total ETBR and GAPDH (Control) protein in 50 μg whole cell lysates. Panel C shows surface ETBR protein expression in live cells as seen by flow cytometry, where the first peak indicates cells treated to secondary detection reagent alone, the middle peak indicates cells untreated with BRAF inhibitor, and the last peak indicates BRAF inhibitor treated cells.

FIG. 5 shows in vivo combination efficacy of anti-ET_{B}R ADC (Hu5E9v1-vc-MMAE) and BRAFi-945 against UACC-257X2.2 melanoma xenograft mouse models at varying doses. Subcutaneous tumors were established in mice inoculated with UACC-257X2.2 cell lines. When tumor volumes reached approximately 200 mm³ (day 0), animals were dosed orally once a day for 21 days with BRAFi-945 or vehicle control. On day 1 (after two doses of BRAFi-945), animals were given a single IV injection of either vehicle or anti-ET_{B}R ADC at the indicated doses. Average tumor volumes with standard deviations were determined from 10 animals per group. Drug and dosage information are as indicated: Panel A shows a 1 mpk BRAFi-945 and 1 mpk anti-ET_{B}R-ADC (Hu5E9v1-vc-MMAE) combination; panel B shows a 1 mpk BRAFi-945 and 3 mpk anti-ET_{B}R-ADC (Hu5E9v1-vc-MMAE) combination; panel C shows a 6 mpk BRAFi-945 and 1 mpk anti-ET_{B}R-ADC (Hu5E9v1-vc-MMAE) combination; panel D shows a 6 mpk BRAFi-945 and 3 mpk anti-ET_{B}R-ADC (Hu5E9v1-vc-MMAE) combination; and panel E shows a 20 mpk BRAFi-945 and 3 mpk anti-ET_{B}R-ADC (Hu5E9v1-vc-MMAE) combination.

FIG. 6 shows ETBR expression in COLO 829 melanoma cells treated for 24 h with varying concentrations of BRAF inhibitor RG7204. Panel A shows ETBR transcript normalized to RPL19 transcript. Panel B shows expression of total ETBR and GAPDH (Control) protein in 50 μg whole cell lysates. Panel C shows surface ETBR protein expression in live cells as seen by flow cytometry, where the first peak indicates cells treated to secondary detection reagent alone, the second peak indicates cells untreated with BRAF inhibitor and the third peak indicates BRAF inhibitor treated cells.

FIG. 7 demonstrates the in vivo combination efficacy of anti-ET_{B}R ADC and BRAF inhibitor RG7204 against COLO 829 melanoma xenografts mouse model. Subcutaneous tumors were established in mice inoculated with COLO 829 melanoma cell lines. When tumor volumes reached approximately 200 mm³ (day 0), animals were dosed orally twice a day for 21 days with RG7204. On day 1 (after three doses of RG7204), animals were given a single IV injection of either vehicle or anti-ET_{B}R ADC (Hu5E9v1-vc-MMAE) at the indicated doses. Average tumor volumes with standard deviations were determined from 9 animals per group. Drug and dosage information are as indicated: panel A shows 3 mpk of anti-ET_{B}R-ADC (Hu5E9v1-vc-MMAE) in combination with 30 mpk of RG7204; panel B shows 1 mpk of anti-ET_{B}R-ADC (Hu5E9v1-vc-MMAE) in combination with 30 mpk of RG7204; panel C shows 1 mpk of anti-ET_{B}R-ADC (Hu5E9v1-vc-MMAE) in combination with 10 mpk of RG7204; and panel D shows 3 mpk of anti-ET_{B}R-ADC (Hu5E9v1-vc-MMAE) in combination with 10 mpk of RG7204.

FIG. 8 shows ETBR expression in A2058 melanoma cells treated for 24 h with varying concentrations of BRAF inhibitor RG7204. Panel A shows ETBR transcript normalized to RPL19 transcript; panel B shows expression of total ETBR and GAPDH (Control) protein in 100 μg whole cell lysates; and panel C shows surface ETBR protein expression in live cells as seen by flow cytometry. The first peak indicates cells treated to secondary detection reagent alone, the second peak indicates cells untreated with BRAF inhibitor, and the third peak indicates BRAF inhibitor treated cells.

FIG. 9 demonstrates in vivo combination efficacy of anti-ET_{B}R ADC (Hu5E9v1-vc-MMAE) and BRAF inhibitor RG7204 against A2058 melanoma xenograft mouse models. Subcutaneous tumors were established in mice inoculated with A2058 melanoma cell lines. When tumor volumes reached approximately 200 mm³ (day 0), animals were dosed orally twice a day for 21 days with RG7204. On day 1 (after three doses of RG7204), animals were given a single IV injection of either vehicle or anti-ET_{B}R ADC (Hu5E9v1-vc-MMAE) at the indicated doses. Average tumor volumes with standard deviations were determined from 10 animals per group. Drug and dosage information indicated on each graph as follows: Panel A shows 6 mpk of anti-ET_{B}R-ADC (Hu5E9v1-vc-MMAE) in combination with 10 mpk of RG7204; panel B shows 6 mpk of anti-ET_{B}R-ADC (Hu5E9v1-vc-MMAE) in combination with 30 mpk of RG7204; panel C shows 3 mpk of anti-ET_{B}R-ADC (Hu5E9v1-vc-MMAE) in combination with 10 mpk of RG7204; and panel D shows 3 mpk of anti-ET_{B}R-ADC (Hu5E9v1-vc-MMAE) in combination with 30 mpk of RG7204.

FIG. 10 shows Western blot experiments performed with BRAFi RG7204 showing expression of total ETBR, Perk and erk proteins and control proteins GAPDH and β-tubulin in 25 to 100 μg whole cell lysates from IPC-298 melanoma cells.

FIG. 11 shows surface ETBR protein expression in IPC-298 live cells as seen by flow cytometry after incubation with 0.1 μM, 1 μM and 10 μM of BRAFi RG7204 (panels A, B and C respectively). The first peak indicates cells treated to secondary detection reagent alone, the second peak indicates BRAF inhibitor treated cells and the third peak indicates cells untreated with BRAF inhibitor.

FIG. 12 shows Western blot experiments performed with MEKi-623 (panel A) and MEKi-973 (panel B) at concentrations of 0 μM, 0.01 μM, 0.1 μM and 1 μM showing expression of total ETBR, Perk and erk proteins and control proteins GAPDH and β-tubulin in 50 μg whole cell lysates from COLO829 melanoma cells.

FIG. 13 shows surface ETBR protein expression in COLO 829 live cells as seen by flow cytometry after incubation with 0.01 μM (panels A and D), 0.1 μM (panels B and E) and 1 μM (panel C and F) of MEKi-623 (panels A, B and C respectively) or MEKi-973 (panels D, E and F respectively). The first peak indicates cells treated to secondary detection reagent alone, the second peak indicates cells untreated with MEK inhibitor and the third peak indicates MEK inhibitor treated cells.

FIG. 14 shows ETBR mRNA expression in A2058 melanoma cells treated for 24 h with varying concentrations of MEKi-623 (panel A) or MEKi-973 (panel B), normalized to RPL19 transcript.

FIG. 15 shows Western blot experiments performed with MEKi-623 (panel A) and MEKi-973 (panel B) at concentrations of 0 μM, 0.01 μM, 0.1 μM and 1 μM showing expression of total ETBR, Perk and erk proteins and control proteins GAPDH and β-tubulin in 50-100 μg whole cell lysates from A2058 melanoma cells.

FIG. 16 shows surface ETBR protein expression in A2058 live cells as seen by flow cytometry after incubation with 0.01 μM (panels A and D), 0.1 μM (panels B and E) and 1 μM (panel C and F) of MEKi-623 (panels A, B and C respectively) or MEKi-973 (panels D, E and F respectively). The first peak indicates cells treated to secondary detection reagent alone, the second peak indicates cells untreated with MEK inhibitor and the third peak indicates MEK inhibitor treated cells.

FIG. 17 demonstrates in vivo combination efficacy of anti-ET_{B}R ADC (Hu5E9v1-vc-MMAE) and MEKi-973 against A2058 melanoma xenograft mouse models. Subcutaneous tumors were established in mice inoculated with A2058 melanoma cell lines. When tumor volumes reached approximately 200 mm³ (day 0), animals were dosed orally once a day for 21 days with MEKi-973. On day 1 (after two doses of MEKi-973), animals were given a single IV injection of either vehicle or anti-ET_{B}R ADC (Hu5E9v1-vc-MMAE) at the indicated doses. Average tumor volumes with standard deviations were determined from 9 animals per group. Drug and dosage information indicated on each graph as follows: Panel A shows 7.5 mpk of anti-gD ADC (control) in combination with 7.5 mpk of MEKi-973 as compared to a vehicle control and anti-gD ADC alone; panel B shows 6 mpk of anti-ET_{B}R-ADC (Hu5E9v1-vc-MMAE) in combination with 7.5 mpk of MEKi-973 as compared to a vehicle control and 7.5 mpk MEKi-973 alone (GDC-0973) or 6 mpk of anti-ET_{B}R-ADC alone.

FIG. 18 shows ET_{B}R transcript expression in SK23-MEL melanoma cells treated for 24 h with varying concentrations of MEKi-623 (panel A) or MEKi-973 (panel B), which were normalized to RPL19 transcript.

FIG. 19 shows Western blot experiments performed with MEKi-623 (panel A) and MEKi-973 (panel B) at concentrations of 0 μM, 0.01 μM, 0.1 μM and 1 μM showing expression of total ETBR, Perk and erk proteins and control proteins GAPDH and β-tubulin in 50 μg whole cell lysates from SK23-MEL melanoma cells.

FIG. 20 shows surface ETBR protein expression in live SK23-MEL cells as seen by flow cytometry after incubation with 0.01 μM (panels A and D), 0.1 μM (panels B and E) and 1 μM (panel C and F) of MEKi-623 (panels A, B and C respectively) or MEKi-973 (panels D, E and F respectively). The first peak indicates cells treated to secondary detection reagent alone, the second peak indicates cells untreated with MEK inhibitor and the third peak indicates MEK inhibitor treated cells.

FIG. 21 demonstrates in vivo combination efficacy of anti-ET_{B}R ADC (Hu5E9v1-vc-MMAE) and MEKi-973 against SK23-MEL melanoma xenograft mouse models. Drug and dosage information indicated on each graph as follows: Panel A shows 6 mpk of anti-gD ADC (control) in combination with 7.5 mpk of MEKi-973 as compared to a vehicle control, 7.5 mpk MEKi-973 and 6 mpk anti-gD ADC alone; panel B shows 6 mpk of anti-ET_{B}R-ADC (Hu5E9v1-vc-MMAE) in combination with 7.5 mpk of MEKi-973 (“Combination”) as compared to a vehicle control and 7.5 mpk MEKi-973 alone or 6 mpk of anti-ET_{B}R-ADC alone. Panel C shows 3 mpk of anti-ET_{B}R-ADC (Hu5E9v1-vc-MMAE) in combination with 3 mpk of MEKi-973 (“Combination”), as compared to a vehicle control, 3 mpk of anti-ET_{B}R-ADC (Hu5E9v1-vc-MMAE) or 3 mpk of MEKi-973. Panel D shows 3 mpk of anti-ET_{B}R-ADC (Hu5E9v1-vc-MMAE) in combination with 7.5 mpk of MEKi-973 (“Combination”) as compared to a vehicle control and 7.5 mpk MEKi-973 alone or 3 mpk of anti-ET_{B}R-ADC alone. Panel E shows 6 mpk of anti-ET_{B}R-ADC (Hu5E9v1-vc-MMAE) in combination with 3 mpk of MEKi-973 (“Combination”) as compared to a vehicle control and 3 mpk MEKi-973 alone or 6 mpk of anti-ET_{B}R-ADC alone.

FIG. 22 shows Western blot experiments performed with MEKi-623 (panel A) and MEKi-973 (panel B) at concentrations of 0 μM, 0.01 μM, 0.1 μM and 1 μM showing expression of total ETBR, Perk and erk proteins and control proteins GAPDH and β-tubulin in 25-100 μg whole cell lysates from IPC-298 melanoma cells.

FIG. 23 shows surface ETBR protein expression in live IPC-298 cells as seen by flow cytometry after incubation with 0.01 μM (panels A and D), 0.1 μM (panels B and E) and 1 μM (panel C and F) of MEKi-623 (panels A, B and C respectively) or MEKi-973 (panels D, E and F respectively). The first peak indicates cells treated to secondary detection reagent alone, the second peak indicates cells untreated with MEK inhibitor and the third peak indicates MEK inhibitor treated cells.

FIG. 24 demonstrates in vivo combination efficacy of anti-ET_{B}R ADC (Hu5E9v1-vc-MMAE) and MEKi-623 against IPC-298 melanoma xenograft mouse models. Drug and dosage information indicated on each graph as follows: Panel A shows 6 mpk of anti-gD ADC (control) in combination with 1 mpk of MEKi-623 as compared to a vehicle control, 1 mpk MEKi-623 and 6 mpk anti-gD ADC alone; panel B shows 6 mpk of anti-ET_{B}R-ADC (Hu5E9v1-vc-MMAE) in combination with 1 mpk of MEKi-623 (“Combination”) as compared to a vehicle control and 1 mpk MEKi-623 alone or 6 mpk of anti-ET_{B}R-ADC alone.

FIG. 25 in vivo combination efficacy of anti-ET_{B}R ADC (Hu5E9v1-vc-MMAE) and MEKi-973 against IPC-298 melanoma xenograft mouse models. Drug and dosage information indicated on each graph as follows: Panel A shows 6 mpk of anti-gD ADC (control) in combination with 7.5 mpk of MEKi-973 as compared to a vehicle control, 7.5 mpk MEKi-973 and 6 mpk anti-gD ADC alone; panel B shows 6 mpk of anti-ET_{B}R-ADC (Hu5E9v1-vc-MMAE) in combination with 7.5 mpk of MEKi-973 (“Combination”) as compared to a vehicle control and 7.5 mpk MEKi-973 alone or 6 mpk of anti-ET_{B}R-ADC alone.

FIG. 26 depicts expression of phosphorylated erk and total erk protein in COLO 829 tumors treated with either vehicle or 30 mpk BRAFi RG7204.

FIG. 27 depicts ETBR transcript expression in COLO 829 tumors treated with BRAFi RG7204 (panel A) and in A2058 tumors treated with MEKi-973 for 3 days (panel B). Panel A shows ETBR transcript normalized to control GAPDH in COLO 829 cell line, in COLO 892 tumors treated with either vehicle control or 10 mpk or 30 mpk RG7204. Panel B shows ETBR transcript normalized to control Hprt1 in A2058 tumors treated with either vehicle control or 5 or 10 mpk of MEKi-973.

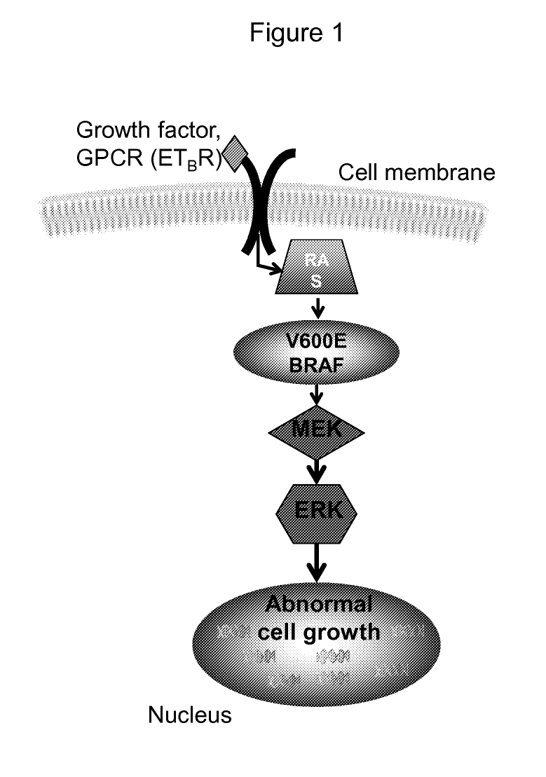

### DETAILED DESCRIPTION OF EMBODIMENTS OF THE INVENTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-ETBR antibody” and “an antibody that binds to ETBR” refer to an antibody that is capable of binding the endothelin B receptor (ETBR) with sufficient affinity such that the antibody is useful as a diagnostic and/or therapeutic agent in targeting ETBR. In one embodiment, the extent of binding of an anti-ETBR antibody to an unrelated, non-ETBR protein is less than about 10% of the binding of the antibody to ETBR as measured, e.g., by a radioimmunoassay (RIA). In certain embodiments, an antibody that binds to ETBR has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M). In certain embodiments, an anti-ETBR antibody binds to an epitope of ETBR that is conserved among ETBR from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “BRAF” as used herein refers to a serine/threonine-protein kinase B-Raf, also known as proto-oncogene B-Raf or v-Raf murine sarcoma viral oncogene homolog B1, which is a protein that in humans is encoded by the BRAF gene. The B-Raf protein is involved in sending signals in cells and in cell growth.

The term “BRAF inhibitor” or “BRAFi” as used herein refers to any number of known small molecule drug compounds which can inhibit or interrupt the B-Raf/MEK step on the B-Raf/MEK/ERK pathway. Examples of suitable BRAFi may include, but are not limited to, those described in International Patent Application PCT/US2010/047007 filed Aug. 27, 2010, in International Patent Application PCT/US2010/046975 filed Aug. 27, 2010; in International Patent Application PCT/US2010/046952 filed Aug. 27, 2010; in International Patent Application PCT/US2010/046955 filed Aug. 27, 2010; and in International Patent Application PCT/US2006/024361 filed Jun. 21, 2006. Another example may be, but is not limited to, GSK 2118436, having a CAS registry number 405554-55-4, which is also known as 5-[2-[4-[2-(Dimethylamino)ethoxy]phenyl]-5-(4-pyridinyl)-1H-imidazol-4-yl]-2,3-dihydro-1H-inden-1-one oxime.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², P²¹², Zr⁸⁹ and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “ETBR,” as used herein, refers to any native endothelin B receptor (ETBR) from any vertebrate source, including mammals such as primates (e.g. humans) and rodents (e.g., mice and rats), unless otherwise indicated. The term encompasses “full-length,” unprocessed ETBR as well as any form of ETBR that results from processing in the cell. The term also encompasses naturally occurring variants of ETBR, e.g., splice variants or allelic variants. The amino acid sequence of an exemplary human ETBR is shown in SEQ ID NO:10 (see Nakamuta M et al., Cloning and Sequence Analysis of a cDNA encoding Human non-selective type of endothelin receptor, Biochem Biophys Res Commun. 1991 May 31:177(1):34-9).

The term “anti-ETBR antibody—ADC” as used herein, refers to any anti-ETBR antibody described herein that is conjugated to a toxin. Such toxins include, but are not limited to, maytansinoids or specifically monomethylauristatin (MMAE). An anti-ETBR antibody-ADC is contemplated as a species of “anti-ETBR antibodies of the invention”.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3 (L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The term “945” or BRAFi-945” as used herein refers to a B-Raf enzyme inhibitor that is 4-amino-N-(6-chloro-2-fluoro-3-(3-fluoro propyl sulfonamido) phenyl)thieno[3,2-d]pyrimidine-7-carboxamide and has a structure having the following formula as disclosed in Example 15 of International Patent Application PCT/US2010/046955 filed Aug. 27, 2010 which is incorporated herein by reference in its entirety:

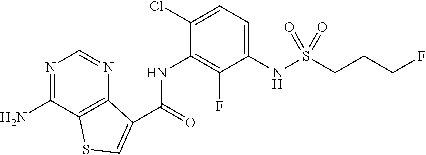 [SMILES: Nc1ncnc2c(C(=O)Nc3c(Cl)ccc(NS(=O)(=O)CCCF)c3F)csc12]

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-ETBR antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “mitogen-activated protein kinase” (MAP kinase) as used herein refers to the serine/threonine-specific protein kinases belonging to the CMGC (CDK/MAPK/GSK3/CLK) kinase group. The ERK1/2 pathway of mammals is probably the best characterized MAPK system. The most important upstream activators of this pathway are the Raf proteins (A-Raf, B-Raf or c-Raf), the key mediators of response to growth factors (EGF, FGF, PDGF, etc.).

The term “MAPK/ERK kinase” (MEK) as used herein refers to a tyrosine kinase which occupies a central role in the MAPK pathway. Expression of constitutively active forms of MEK leads to transformation of cell lines.

The term “MEK inhibitor” (MEKi) as used herein refers to any number of known small molecule drug compounds which can inhibit or interrupt the MEK step on the MAP kinase pathway. Examples of suitable MEKi may include, but are not limited to, those described as MEKi-623, MEKi-973, or GSK1120212.

The term “MEKi-973” as used herein refers to a MEK inhibitor (S)-(3,4-difluoro-2-((2-fluoro-4-iodophenyl)amino)phenyl)(3-hydroxy-3-(piperidin-2yl)azetidin-1-yl)methanone, having the structure:

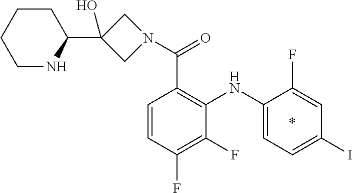 [SMILES: O=C(c1ccc(F)c(F)c1Nc1ccc(I)cc1F)N1CC(O)([C@@H]2CCCCN2)C1]

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “RG7204” refers to a B-Raf enzyme inhibitor that has a molecular formula of C₂₃H₁₈CIF₂N₃O₃S and the following structure:

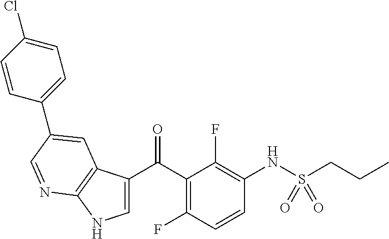 [SMILES: CCCS(=O)(=O)Nc1ccc(F)c(C(=O)c2c[nH]c3ncc(-c4ccc(Cl)cc4)cc23)c1F]

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. Compositions and Methods

In one aspect, the invention is based, in part, on antibodies that bind to ETBR. Antibodies of the invention are useful, e.g., for the treatment of melanoma.

Exemplary Anti-ETBR Antibodies

In one aspect, the invention provides isolated antibodies that bind to ETBR. In certain embodiments, an anti-ETBR antibody comprises at least one, two, three, four, five, or six CDRs selected from (a) CDR-L1 (KSSQSLLDSDGKTYLN, SEQ ID NO:7), (b) CDR-L2 (LVSKLDS, SEQ ID NO:8), (c) CDR-L3 (WQGTHFPYT; SEQ ID NO:9), (d) CDR-H1 (GYTFTSYWMQ; SEQ ID NO:1), (e) CDR-H2 (TIYPGDGDTSYAQKFKG; SEQ ID NO:2), and (f) CDR-H3 (WGYAYDIDN; SEQ ID NO:3).

In any of the above embodiments, an anti-ETBR antibody is humanized. In one embodiment, an anti-ETBR antibody comprises CDRs as in any of the above embodiments, and further comprises an acceptor human framework, e.g. a human immunoglobulin framework or a human consensus framework. In another aspect, the invention provides an isolated anti-ETBR antibody having the VL amino acid sequence of SEQ ID NO:8, and the VH amino acid sequence of SEQ ID NO:7. In yet another aspect, the invention provides an anti-ETBR antibody having a VL sequence of SEQ ID NO:8 and a VH amino acid sequence of SEQ ID NO:9.

In another aspect, an anti-ETBR antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:7 or 9. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-ETBR antibody comprising that sequence retains the ability to bind to ETBR. In certain embodiments, a total of 1 to 10 amino acids have been substituted, inserted and/or deleted in SEQ ID NO:7 or 9. In certain embodiments, substitutions, insertions, or deletions occur in regions outside the CDRs (i.e., in the FRs).

In another aspect, an anti-ETBR antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:8. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-ETBR antibody comprising that sequence retains the ability to bind to ETBR. In certain embodiments, a total of 1 to 10 amino acids have been substituted, inserted and/or deleted in SEQ ID NO:8. In certain embodiments, the substitutions, insertions, or deletions occur in regions outside the HVRs (i.e., in the FRs).

In another aspect, an anti-ETBR antibody is provided, wherein the antibody comprises a VH as in any of the embodiments provided above, and a VL as in any of the embodiments provided above. In one embodiment, the antibody comprises the VH and VL sequences in SEQ ID NO:7 or 9 and SEQ ID NO:8, respectively, including post-translational modifications of those sequences.

In a further aspect, the invention provides an antibody that binds to the same epitope as an anti-ETBR antibody provided herein. For example, in certain embodiments, an antibody is provided that binds to the same epitope as an anti-ETBR antibody comprising a VH sequence of SEQ ID NO:7 or 9 and a VL sequence of SEQ ID NO:8. In certain embodiments, an anti-ETBR antibody is provided that binds to an epitope within an N-terminal extracellular domain #1 fragment of ETBR consisting of amino acids number 64 to 101 of SEQ ID NO:10.

In a further aspect of the invention, an anti-ETBR antibody according to any of the above embodiments is a monoclonal antibody, including a chimeric, humanized or human antibody. In one embodiment, an anti-ETBR antibody is an antibody fragment, e.g., a Fv, Fab, Fab′, scFv, diabody, or F(ab′)₂ fragment. In another embodiment, the antibody is a full length antibody, e.g., an intact IgG1 antibody or other antibody class or isotype as defined herein.

In a further aspect, an anti-ETBR antibody according to any of the above embodiments may incorporate any of the features, singly or in combination, as described in Sections 1-7 below:

Antibody Affinity

In certain embodiments, an antibody provided herein has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al. Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for ETBR and the other is for any other antigen. In certain embodiments, bispecific antibodies may bind to two different epitopes of ETBR. Bispecific antibodies may also be used to localize cytotoxic agents to cells which express ETBR. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to ETBR as well as another, different antigen (see, US 2008/0069820, for example).

Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties: hydrophobic:

### Norleucine, Met, Ala, Val, Leu, Ile;

neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

Recombinant Methods and Compositions

Antibodies may be produced using recombinant methods and compositions, e.g., as described in U.S. Pat. No. 4,816,567. In one embodiment, isolated nucleic acid encoding an anti-ETBR antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-ETBR antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-ETBR antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Assays

Anti-ETBR antibodies provided herein may be identified, screened for, or characterized for their physical/chemical properties and/or biological activities by various assays known in the art.

Binding Assays and Other Assays

In one aspect, an antibody of the invention is tested for its antigen binding activity, e.g., by known methods such as ELISA, Western blot, etc.

In another aspect, competition assays may be used to identify an antibody that competes with, for example, Hu5E9v.1 or Hu5E9v.2 for binding to ETBR. In certain embodiments, such a competing antibody binds to the same epitope (e.g., a linear or a conformational epitope) that is bound by Hu5E9v.1 or Hu5E9v.2. Detailed exemplary methods for mapping an epitope to which an antibody binds are provided in Morris (1996) “Epitope Mapping Protocols,” in Methods in Molecular Biology vol. 66 (Humana Press, Totowa, N.J.). In one aspect of the invention, anti-ETBR antibodies described herein specifically bind an ETBR epitope consisting of amino acids number 64 to 101 of SEQ ID NO:10.

In an exemplary competition assay, immobilized ETBR is incubated in a solution comprising a first labeled antibody that binds to ETBR (e.g., Hu5E9v.1 or Hu5E9v.2) and a second unlabeled antibody that is being tested for its ability to compete with the first antibody for binding to ETBR. The second antibody may be present in a hybridoma supernatant. As a control, immobilized ETBR is incubated in a solution comprising the first labeled antibody but not the second unlabeled antibody. After incubation under conditions permissive for binding of the first antibody to ETBR, excess unbound antibody is removed, and the amount of label associated with immobilized ETBR is measured. If the amount of label associated with immobilized ETBR is substantially reduced in the test sample relative to the control sample, then that indicates that the second antibody is competing with the first antibody for binding to ETBR. See Harlow and Lane (1988) Antibodies: A Laboratory Manual ch. 14 (Cold Spring Harbor Laboratory, Cold Spring Harbor, N.Y.).

Activity Assays

In one aspect, assays are provided for identifying whether anti-ETBR antibodies and/or BRAFi compounds have biological activity. Biological activity may include those described in the Examples, e.g., in vitro melanoma cell survival assays or in vivo xenograft models in which melanoma cell lines are transplanted into nude mice and tumor growth inhibition (TGI) is assessed.

Immunoconjugates

The invention also provides immunoconjugates comprising an anti-ETBR antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

The invention also provides immunoconjugates (interchangeably referred to as “antibody-drug conjugates,” or “ADCs”) comprising an antibody conjugated to one or more cytotoxic agents, such as a chemotherapeutic agent, a drug, a growth inhibitory agent, a toxin (e.g., a protein toxin, an enzymatically active toxin of bacterial, fungal, plant, or animal origin, or fragments thereof), or a radioactive isotope (i.e., a radioconjugate).

Immunoconjugates have been used for the local delivery of cytotoxic agents, i.e., drugs that kill or inhibit the growth or proliferation of cells, in the treatment of cancer (Xie et al (2006) Expert. Opin. Biol. Ther. 6(3):281-291; Kovtun et al (2006) Cancer Res. 66(6):3214-3121; Law et al (2006) Cancer Res. 66(4):2328-2337; Lambert, J. (2005) Curr. Opinion in Pharmacology 5:543-549; Wu et al (2005) Nature Biotechnology 23(9):1137-1146; Payne, G. (2003) i 3:207-212; Syrigos and Epenetos (1999) Anticancer Research 19:605-614; Niculescu-Duvaz and Springer (1997) Adv. Drug Deliv. Rev. 26:151-172; U.S. Pat. No. 4,975,278) Immunoconjugates allow for the targeted delivery of a drug moiety to a tumor, and intracellular accumulation therein, where systemic administration of unconjugated drugs may result in unacceptable levels of toxicity to normal cells as well as the tumor cells sought to be eliminated (Baldwin et al., Lancet (Mar. 15, 1986) pp. 603-05; Thorpe (1985) “Antibody Carriers Of Cytotoxic Agents In Cancer Therapy: A Review,” in Monoclonal Antibodies '84: Biological And Clinical Applications (A. Pinchera et al., eds) pp. 475-506. Both polyclonal antibodies and monoclonal antibodies have been reported as useful in these strategies (Rowland et al., (1986) Cancer Immunol. Immunother. 21:183-87). Drugs used in these methods include daunomycin, doxorubicin, methotrexate, and vindesine (Rowland et al., (1986) supra). Toxins used in antibody-toxin conjugates include bacterial toxins such as diphtheria toxin, plant toxins such as ricin, small molecule toxins such as geldanamycin (Mandler et al (2000) J. Nat. Cancer Inst. 92(19):1573-1581; Mandler et al (2000) Bioorganic & Med. Chem. Letters 10:1025-1028; Mandler et al (2002) Bioconjugate Chem. 13:786-791), maytansinoids (EP 1391213; Liu et al., (1996) Proc. Natl. Acad. Sci. USA 93:8618-8623), and calicheamicin (Lode et al (1998) Cancer Res. 58:2928; Hinman et al (1993) Cancer Res. 53:3336-3342). Efforts to design and refine ADC have focused on the selectivity of monoclonal antibodies (mAbs) as well as drug mechanism of action, drug-linking, drug/antibody ratio (loading), and drug-releasing properties (McDonagh (2006) Protein Eng. Design & Sel.; Doronina et al (2006) Bioconj. Chem. 17:114-124; Erickson et al (2006) Cancer Res. 66(8):1-8; Sanderson et al (2005) Clin. Cancer Res. 11:843-852; Jeffrey et al (2005) J. Med. Chem. 48:1344-1358; Hamblett et al (2004) Clin. Cancer Res. 10:7063-7070). The toxins may exert their cytotoxic effects by mechanisms including tubulin binding, DNA binding, or topoisomerase inhibition. Some cytotoxic drugs tend to be inactive or less active when conjugated to large antibodies or protein receptor ligands.

ZEVALIN® (ibritumomab tiuxetan, Biogen/Idec) is an antibody-radioisotope conjugate composed of a murine IgG1 kappa monoclonal antibody directed against the CD20 antigen found on the surface of normal and malignant B lymphocytes and 111In or 90Y radioisotope bound by a thiourea linker-chelator (Wiseman et al (2000) Eur. Jour. Nucl. Med. 27(7):766-77; Wiseman et al (2002) Blood 99(12):4336-42; Witzig et al (2002) J. Clin. Oncol. 20(10):2453-63; Witzig et al (2002) J. Clin. Oncol. 20(15):3262-69). Although ZEVALIN has activity against B-cell non-Hodgkin's Lymphoma (NHL), administration results in severe and prolonged cytopenias in most patients. MYLOTARG™ (gemtuzumab ozogamicin, Wyeth Pharmaceuticals), an antibody-drug conjugate composed of a huCD33 antibody linked to calicheamicin, was approved in 2000 for the treatment of acute myeloid leukemia by injection (Drugs of the Future (2000) 25(7):686; U.S. Pat. Nos. 4,970,198; 5,079,233; 5,585,089; 5,606,040; 5,693,762; 5,739,116; 5,767,285; 5,773,001). Antibody-drug conjugates (ADCs) composed of the maytansinoid, DM1, linked to trastuzumab show potent anti-tumor activity in HER2-overexpressing trastuzumab-sensitive and -resistant tumor cell lines and xenograft models of human cancer. Trastuzumab-MCC-DM1 (T-DM1) is currently undergoing evaluation in phase II clinical trials in patients whose disease is refractory to HER2-directed therapies (Beeram et al (2007) “A phase I study of trastuzumab-MCC-DM1 (T-DM1), a first-in-class HER2 antibody-drug conjugate (ADC), in patients (pts) with HER2+ metastatic breast cancer (BC)”, American Society of Clinical Oncology 43rd: June 2 (Abs 1042; Krop et al, European Cancer Conference ECCO, Poster 2118, Sep. 23-27, 2007, Barcelona; U.S. Pat. No. 7,097,840; US 2005/0276812; US 2005/0166993). The auristatin peptides, auristatin E (AE) and monomethylauristatin (MMAE), synthetic analogs of dolastatin, were conjugated to chimeric monoclonal antibodies cBR96 (specific to Lewis Y on carcinomas) and cAC10 (specific to CD30 on hematological malignancies) (Doronina et al (2003) Nature Biotechnol. 21(7):778-784) and are under therapeutic development.

In certain embodiments, an immunoconjugate comprises an antibody and a chemotherapeutic agent or other toxin. Chemotherapeutic agents useful in the generation of immunoconjugates are described herein (e.g., above). Enzymatically active toxins and fragments thereof can also be used and are described herein.

In certain embodiments, an immunoconjugate comprises an antibody and one or more small molecule drug moieties (toxins), including, but not limited to, small molecule drugs such as a calicheamicin, maytansinoid, dolastatin, auristatin, anthracycline, taxane, trichothecene, and CC1065, and the derivatives of these drugs that have cytotoxic activity. Examples of such immunoconjugates are discussed in further detail below.

Exemplary Immunoconjugates

An immunoconjugate (or “antibody-drug conjugate” (“ADC”)) of the invention may be of Formula I, below, wherein an antibody is conjugated (i.e., covalently attached) to one or more drug moieties (D) through an optional linker (L).

Ab-(L-D)ₚ I

Accordingly, the antibody may be conjugated to the drug either directly or via a linker. In Formula I, p is the average number of drug moieties per antibody, which can range, e.g., from about 1 to about 20 drug moieties per antibody, and in certain embodiments, from 1 to about 8 drug moieties per antibody.

Exemplary Linkers

A linker may comprise one or more linker components. Exemplary linker components include 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate (“SPP”), N-succinimidyl 4-(N-maleimidomethyl)cyclohexane-1 carboxylate (“SMCC”), and N-Succinimidyl (4-iodo-acetyl)aminobenzoate (“SIAB”). Various linker components are known in the art, some of which are described below.

A linker may be a “cleavable linker,” facilitating release of a drug in the cell. For example, an acid-labile linker (e.g., hydrazone), protease-sensitive (e.g., peptidase-sensitive) linker, photolabile linker, dimethyl linker or disulfide-containing linker (Chari et al., Cancer Research 52:127-131 (1992); U.S. Pat. No. 5,208,020) may be used.

In certain embodiments, a linker is as shown in the following Formula II:

-Aₐ-W_{w}-Y_{y}- II

wherein A is a stretcher unit, and a is an integer from 0 to 1; W is an amino acid unit, and w is an integer from 0 to 12; Y is a spacer unit, and y is 0, 1, or 2; and Ab, D, and p are defined as above for Formula I. Exemplary embodiments of such linkers are described in US 2005-0238649 A1, which is expressly incorporated herein by reference.

In some embodiments, a linker component may comprise a “stretcher unit” that links an antibody to another linker component or to a drug moiety. Exemplary stretcher units are shown below (wherein the wavy line indicates sites of covalent attachment to an antibody):

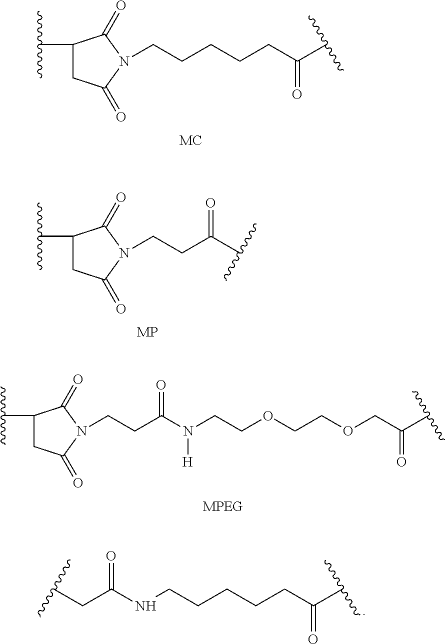 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

In some embodiments, a linker component may comprise an amino acid unit. In one such embodiment, the amino acid unit allows for cleavage of the linker by a protease, thereby facilitating release of the drug from the immunoconjugate upon exposure to intracellular proteases, such as lysosomal enzymes. See, e.g., Doronina et al. (2003) Nat. Biotechnol. 21:778-784. Exemplary amino acid units include, but are not limited to, a dipeptide, a tripeptide, a tetrapeptide, and a pentapeptide. Exemplary dipeptides include: valine-citrulline (vc or val-cit), alanine-phenylalanine (af or ala-phe); phenylalanine-lysine (fk or phe-lys); or N-methyl-valine-citrulline (Me-val-cit). Exemplary tripeptides include: glycine-valine-citrulline (gly-val-cit) and glycine-glycine-glycine (gly-gly-gly). An amino acid unit may comprise amino acid residues that occur naturally, as well as minor amino acids and non-naturally occurring amino acid analogs, such as citrulline Amino acid units can be designed and optimized in their selectivity for enzymatic cleavage by a particular enzyme, for example, a tumor-associated protease, cathepsin B, C and D, or a plasmin protease.

In some embodiments, a linker component may comprise a “spacer” unit that links the antibody to a drug moiety, either directly or by way of a stretcher unit and/or an amino acid unit. A spacer unit may be “self-immolative” or a “non-self-immolative.” A “non-self-immolative” spacer unit is one in which part or all of the spacer unit remains bound to the drug moiety upon enzymatic (e.g., proteolytic) cleavage of the ADC. Examples of non-self-immolative spacer units include, but are not limited to, a glycine spacer unit and a glycine-glycine spacer unit. Other combinations of peptidic spacers susceptible to sequence-specific enzymatic cleavage are also contemplated. For example, enzymatic cleavage of an ADC containing a glycine-glycine spacer unit by a tumor-cell associated protease would result in release of a glycine-glycine-drug moiety from the remainder of the ADC. In one such embodiment, the glycine-glycine-drug moiety is then subjected to a separate hydrolysis step in the tumor cell, thus cleaving the glycine-glycine spacer unit from the drug moiety.

A “self-immolative” spacer unit allows for release of the drug moiety without a separate hydrolysis step. In certain embodiments, a spacer unit of a linker comprises a p-aminobenzyl unit. In one such embodiment, a p-aminobenzyl alcohol is attached to an amino acid unit via an amide bond, and a carbamate, methylcarbamate, or carbonate is made between the benzyl alcohol and a cytotoxic agent. See, e.g., Hamann et al. (2005) Expert Opin. Ther. Patents (2005) 15:1087-1103. In one embodiment, the spacer unit is p-aminobenzyloxycarbonyl (PAB). In certain embodiments, the phenylene portion of a p-amino benzyl unit is substituted with Qm, wherein Q is —C₁-C₈ alkyl, —O—(C₁-C₈ alkyl), -halogen, -nitro or -cyano; and m is an integer ranging from 0-4. Examples of self-immolative spacer units further include, but are not limited to, aromatic compounds that are electronically similar to p-aminobenzyl alcohol (see, e.g., US 2005/0256030 A1), such as 2-aminoimidazol-5-methanol derivatives (Hay et al. (1999) Bioorg. Med. Chem. Lett. 9:2237) and ortho- or para-aminobenzylacetals. Spacers can be used that undergo cyclization upon amide bond hydrolysis, such as substituted and unsubstituted 4-aminobutyric acid amides (Rodrigues et al., Chemistry Biology, 1995, 2, 223); appropriately substituted bicyclo[2.2.1] and bicyclo[2.2.2] ring systems (Storm, et al., J. Amer. Chem. Soc., 1972, 94, 5815); and 2-aminophenylpropionic acid amides (Amsberry, et al., J. Org. Chem., 1990, 55, 5867). Elimination of amine-containing drugs that are substituted at the a-position of glycine (Kingsbury, et al., J. Med. Chem., 1984, 27, 1447) are also examples of self-immolative spacers useful in ADCs.

In one embodiment, a spacer unit is a branched bis(hydroxymethyl)styrene (BHMS) unit as depicted below, which can be used to incorporate and release multiple drugs.

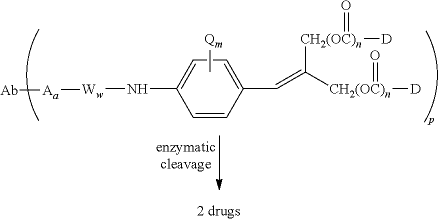

wherein Q is —C₁-C₈ alkyl, —O—(C₁-C₈ alkyl), -halogen, -nitro or -cyano; m is an integer ranging from 0-4; n is 0 or 1; and p ranges raging from 1 to about 20.

In another embodiment, linker L may be a dendritic type linker for covalent attachment of more than one drug moiety through a branching, multifunctional linker moiety to an antibody (Sun et al (2002) Bioorganic & Medicinal Chemistry Letters 12:2213-2215; Sun et al (2003) Bioorganic & Medicinal Chemistry 11:1761-1768). Dendritic linkers can increase the molar ratio of drug to antibody, i.e. loading, which is related to the potency of the ADC. Thus, where a cysteine engineered antibody bears only one reactive cysteine thiol group, a multitude of drug moieties may be attached through a dendritic linker.

Exemplary linker components and combinations thereof are shown below in the context of ADCs of Formula II:

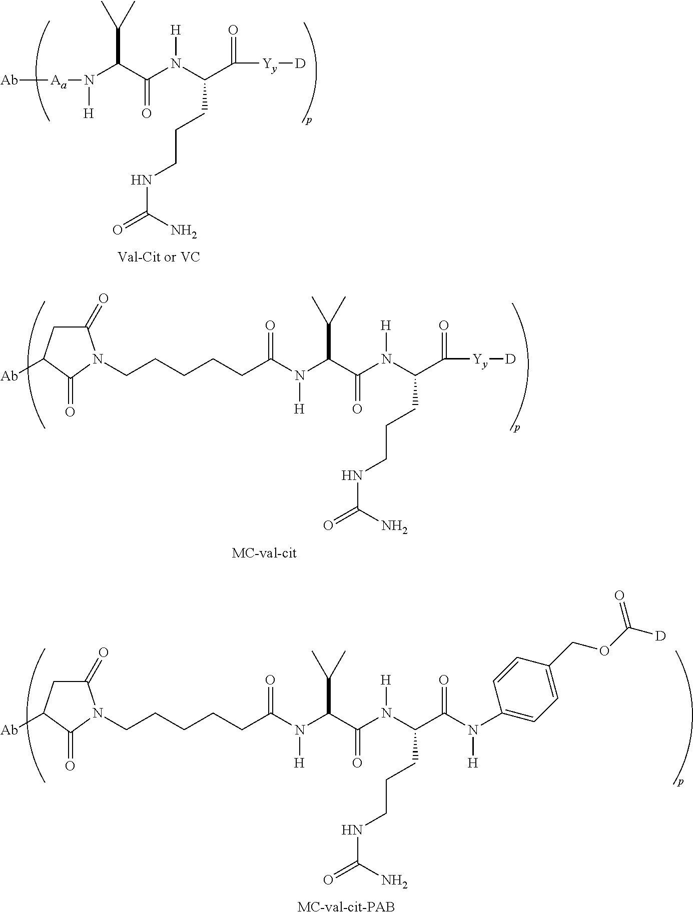

Linkers components, including stretcher, spacer, and amino acid units, may be synthesized by methods known in the art, such as those described in US 2005-0238649 A1.

Exemplary Drug Moieties

Maytansine and Maytansinoids

In some embodiments, an immunoconjugate comprises an antibody conjugated to one or more maytansinoid molecules. Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

Maytansinoid drug moieties are attractive drug moieties in antibody-drug conjugates because they are: (i) relatively accessible to prepare by fermentation or chemical modification or derivatization of fermentation products, (ii) amenable to derivatization with functional groups suitable for conjugation through non-disulfide linkers to antibodies, (iii) stable in plasma, and (iv) effective against a variety of tumor cell lines.

Maytansine compounds suitable for use as maytansinoid drug moieties are well known in the art and can be isolated from natural sources according to known methods or produced using genetic engineering techniques (see Yu et al (2002) PNAS 99:7968-7973). Maytansinol and maytansinol analogues may also be prepared synthetically according to known methods.

Exemplary maytansinoid drug moieties include those having a modified aromatic ring, such as: C-19-dechloro (U.S. Pat. No. 4,256,746) (prepared by lithium aluminum hydride reduction of ansamytocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides). and those having modifications at other positions.

Exemplary maytansinoid drug moieties also include those having modifications such as: C-9-SH (U.S. Pat. No. 4,424,219) (prepared by the reaction of maytansinol with H₂S or P₂S₅); C-14-alkoxymethyl(demethoxy/CH₂OR) (U.S. Pat. No. 4,331,598); C-14-hydroxymethyl or acyloxymethyl (CH₂OH or CH₂OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudlflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

Many positions on maytansine compounds are known to be useful as the linkage position, depending upon the type of link. For example, for forming an ester linkage, the C-3 position having a hydroxyl group, the C-14 position modified with hydroxymethyl, the C-15 position modified with a hydroxyl group and the C-20 position having a hydroxyl group are all suitable.

Maytansinoid drug moieties include those having the structure:

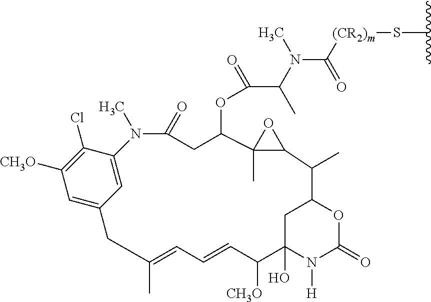

where the wavy line indicates the covalent attachment of the sulfur atom of the maytansinoid drug moiety to a linker of an ADC. R may independently be H or a C₁-C₆ alkyl. The alkylene chain attaching the amide group to the sulfur atom may be methanyl, ethanyl, or propyl, i.e., m is 1, 2, or 3 (U.S. Pat. No. 6,334,10; U.S. Pat. No. 5,208,020; Chari et al (1992) Cancer Res. 52:127-131; Liu et al (1996) Proc. Natl. Acad. Sci USA 93:8618-8623).

All stereoisomers of the maytansinoid drug moiety are contemplated for the compounds of the invention, i.e. any combination of R and S configurations at the chiral carbons of D (U.S. Pat. No. 7,276,497; U.S. Pat. No. 6,913,748; U.S. Pat. No. 6,441,163; U.S. Pat. No. 6,334,10 (RE39151); U.S. Pat. No. 5,208,020; Widdison et al (2006) J. Med. Chem. 49:4392-4408, which are incorporated by reference in their entirety). In one embodiment, the maytansinoid drug moiety will have the following stereochemistry:

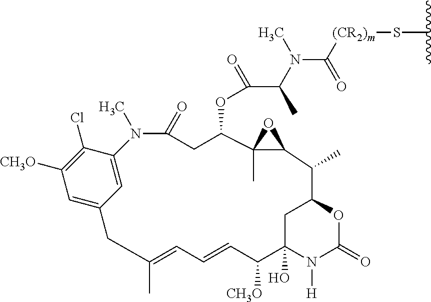

Exemplary embodiments of maytansinoid drug moieties include: DM1; DM3; and DM4, having the structures:

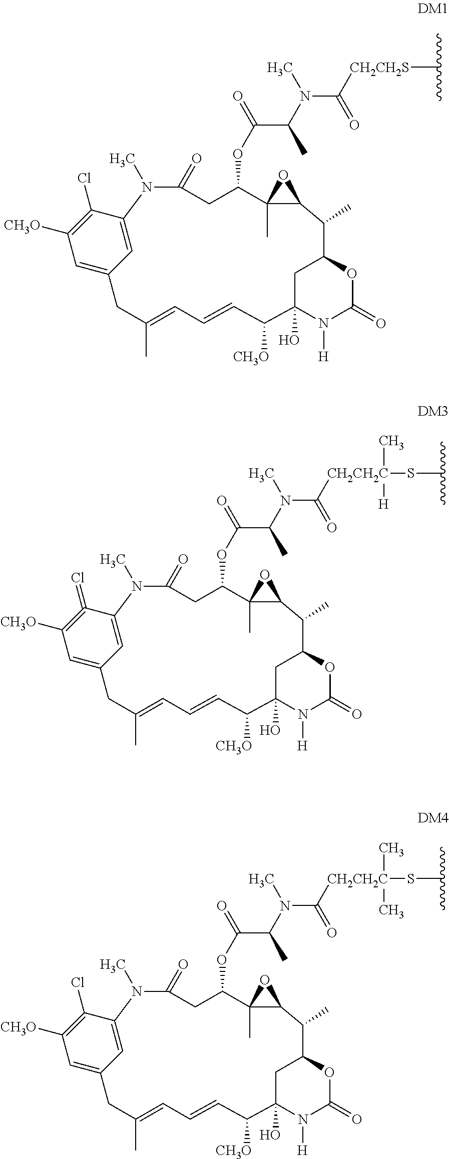

wherein the wavy line indicates the covalent attachment of the sulfur atom of the drug to a linker (L) of an antibody-drug conjugate. HERCEPTIN® (trastuzumab) linked by SMCC to DM1 has been reported (WO 2005/037992; US 2005/0276812; US 2005/016993).

Other exemplary maytansinoid antibody-drug conjugates have the following structures and abbreviations, (wherein Ab is antibody and p is 1 to about 8):

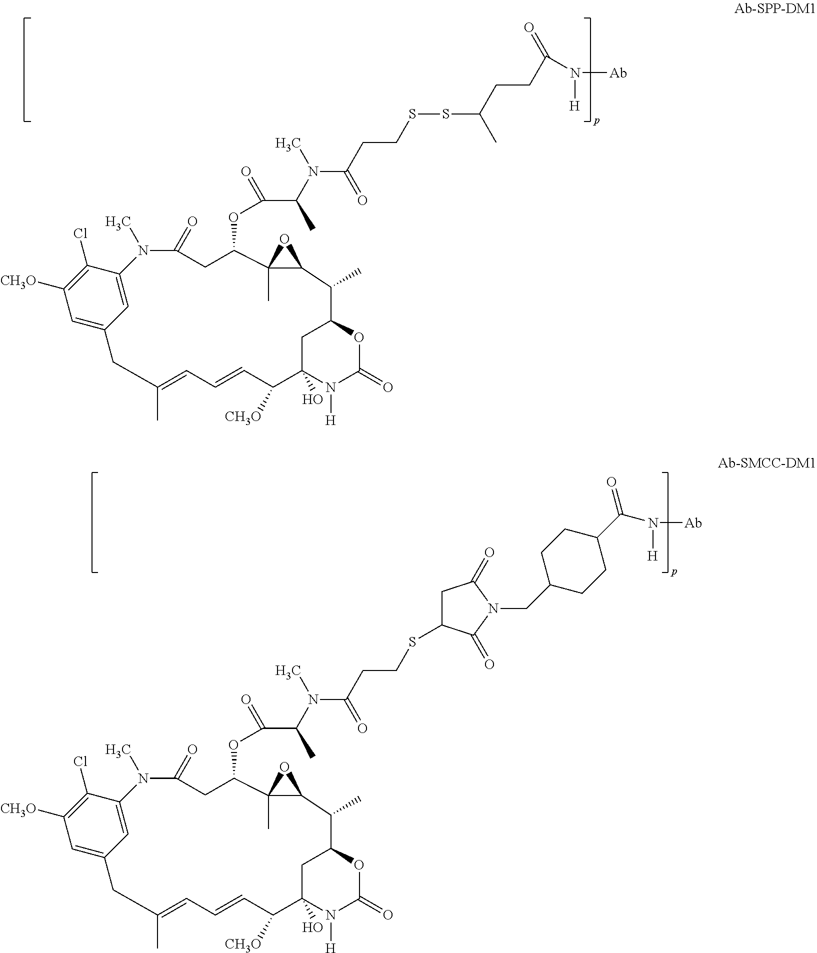

Exemplary antibody-drug conjugates where DM1 is linked through a BMPEO linker to a thiol group of the antibody have the structure and abbreviation:

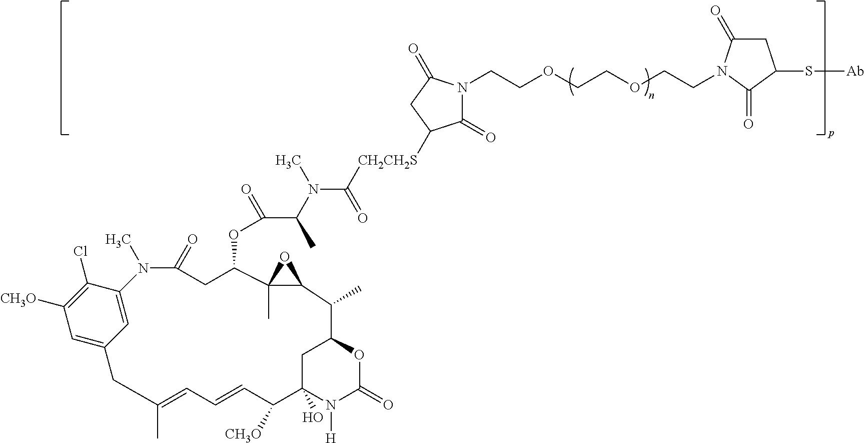

where Ab is antibody; n is 0, 1, or 2; and p is 1, 2, 3, or 4.

Immunoconjugates containing maytansinoids, methods of making the same, and their therapeutic use are disclosed, for example, in U.S. Pat. Nos. 5,208,020, 5,416,064, US 2005/0276812 A1, and European Patent EP 0 425 235 B1, the disclosures of which are hereby expressly incorporated by reference. Liu et al. Proc. Natl. Acad. Sci. USA 93:8618-8623 (1996) describe immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al. Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene. The cytotoxicity of the TA.1-maytansonoid conjugate was tested in vitro on the human breast cancer cell line SK-BR-3, which expresses 3×10⁵ HER-2 surface antigens per cell. The drug conjugate achieved a degree of cytotoxicity similar to the free maytansinoid drug, which could be increased by increasing the number of maytansinoid molecules per antibody molecule. The A7-maytansinoid conjugate showed low systemic cytotoxicity in mice.

Antibody-maytansinoid conjugates are prepared by chemically linking an antibody to a maytansinoid molecule without significantly diminishing the biological activity of either the antibody or the maytansinoid molecule. See, e.g., U.S. Pat. No. 5,208,020 (the disclosure of which is hereby expressly incorporated by reference). An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Preferred maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1; Chari et al. Cancer Research 52:127-131 (1992); and US 2005/016993 A1, the disclosures of which are hereby expressly incorporated by reference. Antibody-maytansinoid conjugates comprising the linker component SMCC may be prepared as disclosed in US 2005/0276812 A1, “Antibody-drug conjugates and Methods.” The linkers comprise disulfide groups, thioether groups, acid labile groups, photolabile groups, peptidase labile groups, or esterase labile groups, as disclosed in the above-identified patents. Additional linkers are described and exemplified herein.

Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In certain embodiments, the coupling agent is N-succinimidyl-3-(2-pyridyldithio) propionate (SPDP) (Carlsson et al., Biochem. J. 173:723-737 (1978)) or N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. In one embodiment, the linkage is formed at the C-3 position of maytansinol or a maytansinol analogue.

Auristatins and Dolastatins

In some embodiments, an immunoconjugate comprises an antibody conjugated to dolastatin or a dolastatin peptidic analog or derivative, e.g., an auristatin (U.S. Pat. Nos. 5,635,483; 5,780,588). Dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin or auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF (U.S. Pat. No. 7,498,298).

A peptidic drug moiety may be selected from Formulas D_{E} and D_{F} below:

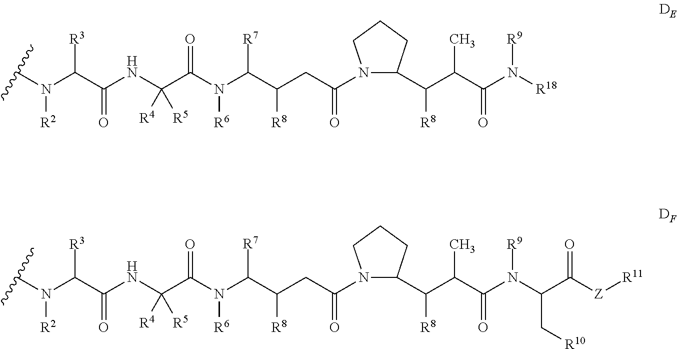

wherein the wavy line of D_{E} and D_{F} indicates the covalent attachment site to an antibody or antibody-linker component, and independently at each location:

R² is selected from H and C₁-C₈ alkyl;

R³ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);

R⁴ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);

R⁵ is selected from H and methyl; or R⁴ and R⁵ jointly form a carbocyclic ring and have the formula —(CR^{a}R^{b})ₙ— wherein R^{a} and R^{b} are independently selected from H, C₁-C₈ alkyl and C₃-C₈ carbocycle and n is selected from 2, 3, 4, 5 and 6;

R⁶ is selected from H and C₁-C₈ alkyl;

R⁷ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);

each R⁸ is independently selected from H, OH, C₁-C₈ alkyl, C₃-C₈ carbocycle and O—(C₁-C₈ alkyl);

R⁹ is selected from H and C₁-C₈ alkyl;

R¹⁰ is selected from aryl or C₃-C₈ heterocycle;

Z is O, S, NH, or NR¹², wherein R¹² is C₁-C₈ alkyl;

R¹¹ is selected from H, C₁-C₂₀ alkyl, aryl, C₃-C₈ heterocycle, —(R¹³O)ₘ—R¹⁴, or —(R¹³O)ₘ—CH(R¹⁵)₂;

m is an integer ranging from 1-1000;

R¹³ is C₂-C₈ alkyl;

R¹⁴ is H or C₁-C₈ alkyl;

each occurrence of R¹⁵ is independently H, COOH, —(CH₂)ₙ—N(R¹⁶)₂, —(CH₂)ₙ—SO₃H, or —(CH₂)ₙ—SO₃—C₁-C₈ alkyl;

each occurrence of R¹⁶ is independently H, C₁-C₈ alkyl, or —(CH₂)ₙ—COOH;

R¹⁸ is selected from —C(R⁸)₂—C(R⁸)₂-aryl, —C(R⁸)₂—C(R⁸)₂—(C₃-C₈ heterocycle), and —C(R⁸)₂—C(R⁸)₂—(C₃-C₈ carbocycle); and n is an integer ranging from 0 to 6.

In one embodiment, R³, R⁴ and R⁷ are independently isopropyl or sec-butyl and R⁵ is —H or methyl. In an exemplary embodiment, R³ and R⁴ are each isopropyl, R⁵ is —H, and R⁷ is sec-butyl. In yet another embodiment, R² and R⁶ are each methyl, and R⁹ is —H. In still another embodiment, each occurrence of R⁸ is —OCH₃. In an exemplary embodiment, R³ and R⁴ are each isopropyl, R² and R⁶ are each methyl, R⁵ is —H, R⁷ is sec-butyl, each occurrence of R⁸ is —OCH₃, and R⁹ is —H. In one embodiment, Z is —O— or —NH—. In one embodiment, R¹⁰ is aryl. In an exemplary embodiment, R¹⁰ is -phenyl. In an exemplary embodiment, when Z is —O—, R¹¹ is —H, methyl or t-butyl. In one embodiment, when Z is —NH, R¹¹ is —CH(R¹⁵)₂, wherein R¹⁵ is —(CH₂)ₙ—N(R¹⁶)₂, and R¹⁶ is —C₁-C₈ alkyl or —(CH₂)ₙ—COOH. In another embodiment, when Z is —NH, R¹¹ is —CH(R¹⁵)₂, wherein R¹⁵ is —(CH₂)ₙ—SO₃H.

An exemplary auristatin embodiment of formula D_{E} is MMAE, wherein the wavy line indicates the covalent attachment to a linker (L) of an antibody-drug conjugate:

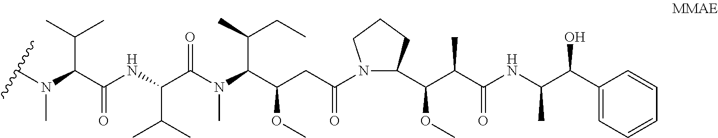 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

An exemplary auristatin embodiment of formula D_{F} is MMAF, wherein the wavy line indicates the covalent attachment to a linker (L) of an antibody-drug conjugate (see U.S. Pat. No. 7,498,298 and Doronina et al. (2006) Bioconjugate Chem. 17:114-124):

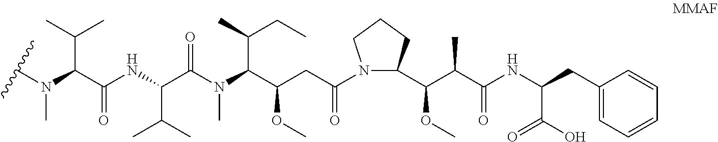 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

Other exemplary embodiments include monomethylvaline compounds having phenylalanine carboxy modifications at the C-terminus of the pentapeptide auristatin drug moiety (WO 2007/008848) and monomethylvaline compounds having phenylalanine sidechain modifications at the C-terminus of the pentapeptide auristatin drug moiety (WO 2007/008603).

Other drug moieties include the following MMAF derivatives, wherein the wavy line indicates the covalent attachment to a linker (L) of an antibody-drug conjugate:

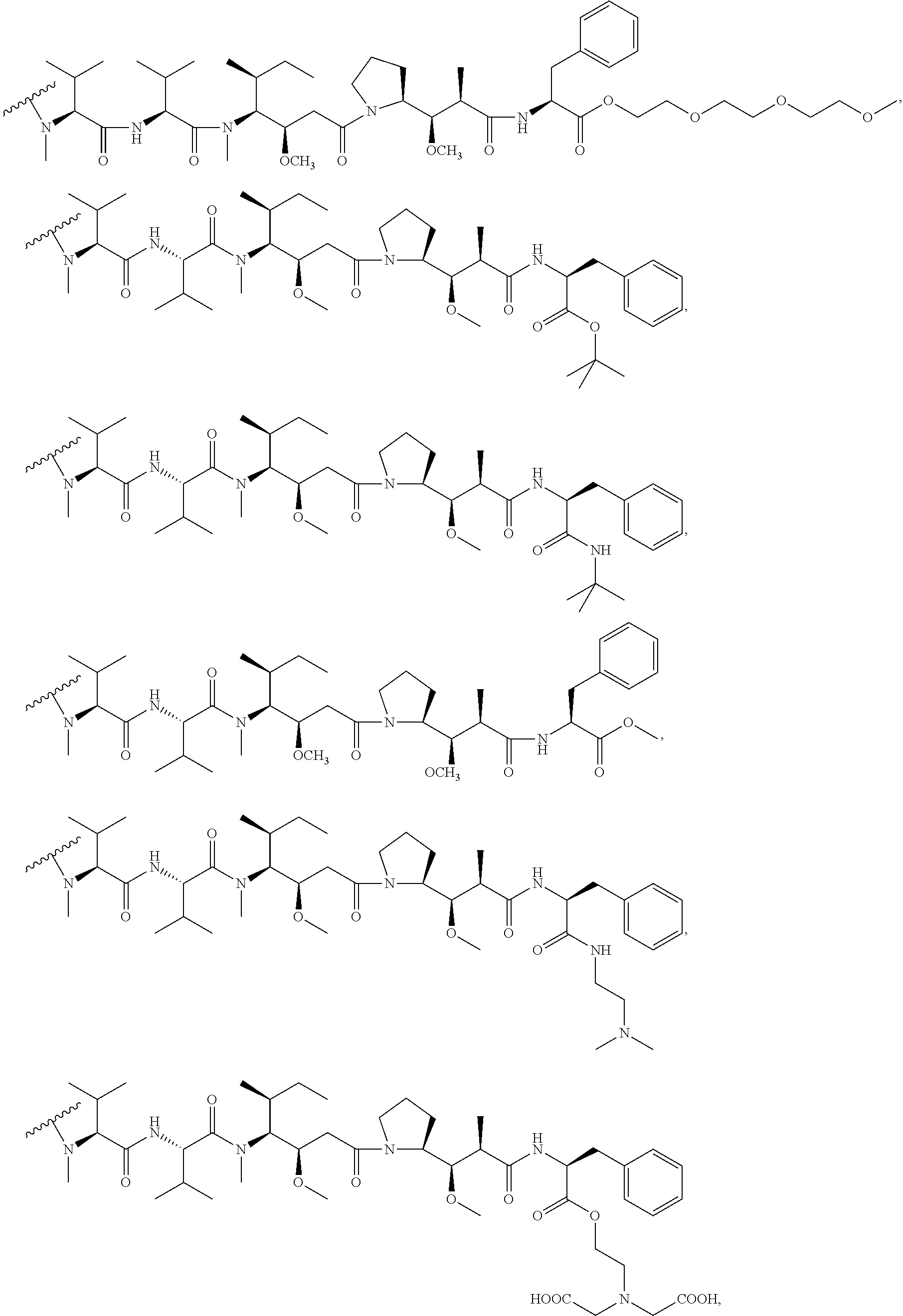

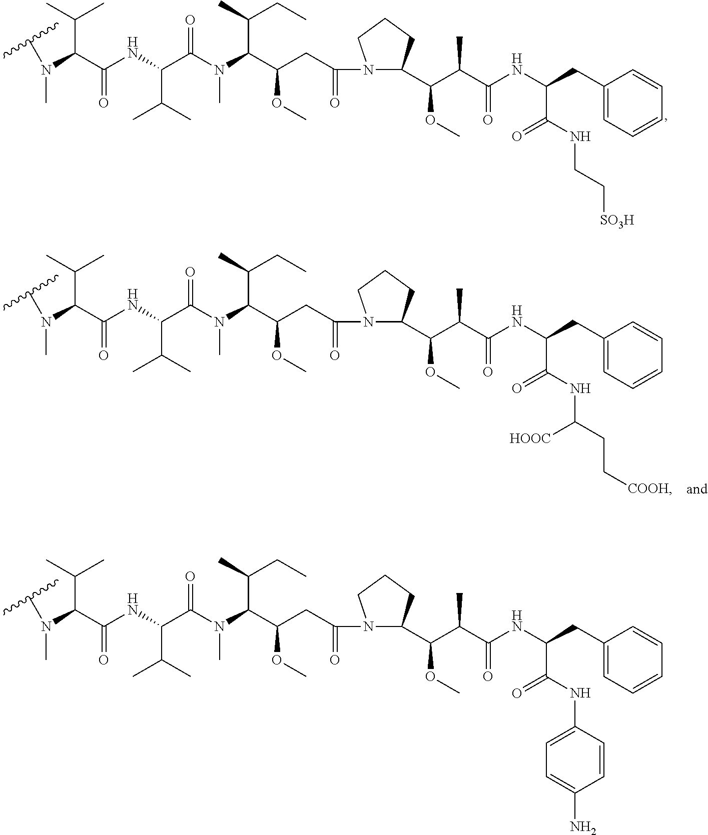 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)NCCS(=O)(=O)O)OC)C(C)C)C(C)C ; *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)NC(CCC(=O)O)C(=O)O)OC)C(C)C)C(C)C ; "and" ; *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)Nc1ccc(N)cc1)OC)C(C)C)C(C)C]

In one aspect, hydrophilic groups including but not limited to, triethylene glycol esters (TEG), as shown above, can be attached to the drug moiety at R¹¹. Without being bound by any particular theory, the hydrophilic groups assist in the internalization and non-agglomeration of the drug moiety.

Exemplary embodiments of ADCs of Formula I comprising an auristatin/dolastatin or derivative thereof are described in U.S. Pat. No. 7,498,298 and Doronina et al. (2006) Bioconjugate Chem. 17:114-124, which is expressly incorporated herein by reference. Exemplary embodiments of ADCs of Formula I comprising MMAE or MMAF and various linker components have the following structures and abbreviations (wherein “Ab” is an antibody; p is 1 to about 8, “Val-Cit” is a valine-citrulline dipeptide; and “S” is a sulfur atom:

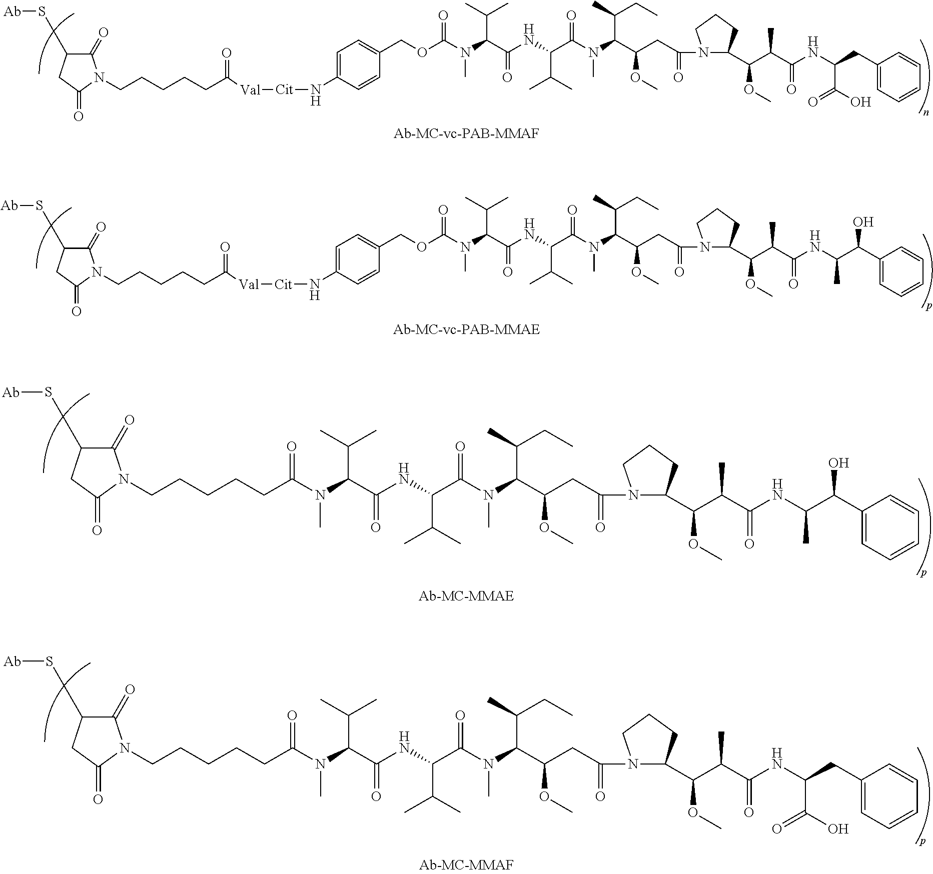

Exemplary embodiments of ADCs of Formula I comprising MMAF and various linker components further include Ab-MC-PAB-MMAF and Ab-PAB-MMAF. Interestingly, immunoconjugates comprising MMAF attached to an antibody by a linker that is not proteolytically cleavable have been shown to possess activity comparable to immunoconjugates comprising MMAF attached to an antibody by a proteolytically cleavable linker. See, Doronina et al. (2006) Bioconjugate Chem. 17:114-124. In such instances, drug release is believed to be effected by antibody degradation in the cell. Id.

Typically, peptide-based drug moieties can be prepared by forming a peptide bond between two or more amino acids and/or peptide fragments. Such peptide bonds can be prepared, for example, according to the liquid phase synthesis method (see E. Schröder and K. Lübke, “The Peptides”, volume 1, pp 76-136, 1965, Academic Press) that is well known in the field of peptide chemistry. Auristatin/dolastatin drug moieties may be prepared according to the methods of: US 2005-0238649 A1; U.S. Pat. No. 5,635,483; U.S. Pat. No. 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat. Biotechnol. 21(7):778-784.

In particular, auristatin/dolastatin drug moieties of formula D_{F}, such as MMAF and derivatives thereof, may be prepared using methods described in U.S. Pat. No. 7,498,298 and Doronina et al. (2006) Bioconjugate Chem. 17:114-124. Auristatin/dolastatin drug moieties of formula D_{E}, such as MMAE and derivatives thereof, may be prepared using methods described in Doronina et al. (2003) Nat. Biotech. 21:778-784. Drug-linker moieties MC-MMAF, MC-MMAE, MC-vc-PAB-MMAF, and MC-vc-PAB-MMAE may be conveniently synthesized by routine methods, e.g., as described in Doronina et al. (2003) Nat. Biotech. 21:778-784, and U.S. Pat. No. 7,498,298, and then conjugated to an antibody of interest.

Calicheamicin

In other embodiments, the immunoconjugate comprises an antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ^{I}₁ (Hinman et al., Cancer Research 53:3336-3342 (1993), Lode et al., Cancer Research 58:2925-2928 (1998), and the aforementioned U.S. patents to American Cyanamid). Another anti-tumor drug to which the antibody can be conjugated is QFA, which is an antifolate. Both calicheamicin and QFA have intracellular sites of action and do not readily cross the plasma membrane. Therefore, cellular uptake of these agents through antibody-mediated internalization greatly enhances their cytotoxic effects.

Other Cytotoxic Agents

Other antitumor agents that can be conjugated to an antibody include anthracyclines (Kratz et al (2006) Current Med. Chem. 13:477-523; Jeffrey et al (2006) Bioorganic & Med. Chem. Letters 16:358-362; Torgov et al (2005) Bioconj. Chem. 16:717-721; Nagy et al (2000) Proc. Natl. Acad. Sci. 97:829-834; Dubowchik et al (2002) Bioorg. & Med. Chem. Letters 12:1529-1532; King et al (2002) J. Med. Chem. 45:4336-4343; U.S. Pat. No. 6,630,579), BCNU, streptozocin, vincristine and 5-fluorouracil, the family of agents known collectively as the LL-E33288 complex, described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an antibody and a compound with nucleolytic activity (e.g., a ribonuclease or a DNA endonuclease such as a deoxyribonuclease; DNase).

In certain embodiments, an immunoconjugate may comprise a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², P²¹², Zr⁸⁹ and radioactive isotopes of Lu. When the immunoconjugate is used for detection, it may comprise a radioactive atom for scintigraphic studies, for example tc^{99m} or I¹²³, or a spin label for nuclear magnetic resonance (NMR) imaging (also known as magnetic resonance imaging, mri), such as iodine-123, iodine-131, indium-111, fluorine-19, carbon-13, nitrogen-15, oxygen-17, gadolinium, manganese or iron.

The radio- or other labels may be incorporated in the immunoconjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶, Re¹⁸⁸Zr⁸⁹ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

In certain embodiments, an immunoconjugate may comprise an antibody conjugated to a prodrug-activating enzyme that converts a prodrug (e.g., a peptidyl chemotherapeutic agent, see WO 81/01145) to an active drug, such as an anti-cancer drug. Such immunoconjugates are useful in antibody-dependent enzyme-mediated prodrug therapy (“ADEPT”). Enzymes that may be conjugated to an antibody include, but are not limited to, alkaline phosphatases, which are useful for converting phosphate-containing prodrugs into free drugs; arylsulfatases, which are useful for converting sulfate-containing prodrugs into free drugs; cytosine deaminase, which is useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. Enzymes may be covalently bound to antibodies by recombinant DNA techniques well known in the art. See, e.g., Neuberger et al., Nature 312:604-608 (1984).

Drug Loading

Drug loading is represented by p, the average number of drug moieties per antibody in a molecule of Formula I. Drug loading may range from 1 to 20 drug moieties (D) per antibody. ADCs of Formula I include collections of antibodies conjugated with a range of drug moieties, from 1 to 20. The average number of drug moieties per antibody in preparations of ADC from conjugation reactions may be characterized by conventional means such as mass spectroscopy, ELISA assay, and HPLC. The quantitative distribution of ADC in terms of p may also be determined. In some instances, separation, purification, and characterization of homogeneous ADC where p is a certain value from ADC with other drug loadings may be achieved by means such as reverse phase HPLC or electrophoresis.

For some antibody-drug conjugates, p may be limited by the number of attachment sites on the antibody. For example, where the attachment is a cysteine thiol, as in the exemplary embodiments above, an antibody may have only one or several cysteine thiol groups, or may have only one or several sufficiently reactive thiol groups through which a linker may be attached. In certain embodiments, higher drug loading, e.g. p>5, may cause aggregation, insolubility, toxicity, or loss of cellular permeability of certain antibody-drug conjugates. In certain embodiments, the drug loading for an ADC of the invention ranges from 1 to about 8; from about 2 to about 6; or from about 3 to about 5. Indeed, it has been shown that for certain ADCs, the optimal ratio of drug moieties per antibody may be less than 8, and may be about 2 to about 5. See US 2005-0238649 A1.

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, and (iii) partial or limiting reductive conditions for cysteine thiol modification.

It is to be understood that where more than one nucleophilic group reacts with a drug-linker intermediate or linker reagent followed by drug moiety reagent, then the resulting product is a mixture of ADC compounds with a distribution of one or more drug moieties attached to an antibody. The average number of drugs per antibody may be calculated from the mixture by a dual ELISA antibody assay, which is specific for antibody and specific for the drug. Individual ADC molecules may be identified in the mixture by mass spectroscopy and separated by HPLC, e.g. hydrophobic interaction chromatography (see, e.g., McDonagh et al (2006) Prot. Engr. Design & Selection 19(7):299-307; Hamblett et al (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value may be isolated from the conjugation mixture by electrophoresis or chromatography.

Certain Methods of Preparing Immunconjugates

An ADC of Formula I may be prepared by several routes employing organic chemistry reactions, conditions, and reagents known to those skilled in the art, including: (1) reaction of a nucleophilic group of an antibody with a bivalent linker reagent to form Ab-L via a covalent bond, followed by reaction with a drug moiety D; and (2) reaction of a nucleophilic group of a drug moiety with a bivalent linker reagent, to form D-L, via a covalent bond, followed by reaction with a nucleophilic group of an antibody. Exemplary methods for preparing an ADC of Formula I via the latter route are described in US 2005-0238649 A1, which is expressly incorporated herein by reference.

Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent such as DTT (dithiothreitol) or tricarbonylethylphosphine (TCEP), such that the antibody is fully or partially reduced. Each cysteine bridge will thus form, theoretically, two reactive thiol nucleophiles. Additional nucleophilic groups can be introduced into antibodies through modification of lysine residues, e.g., by reacting lysine residues with 2-iminothiolane (Traut's reagent), resulting in conversion of an amine into a thiol. Reactive thiol groups may be introduced into an antibody by introducing one, two, three, four, or more cysteine residues (e.g., by preparing variant antibodies comprising one or more non-native cysteine amino acid residues).

Antibody-drug conjugates of the invention may also be produced by reaction between an electrophilic group on an antibody, such as an aldehyde or ketone carbonyl group, with a nucleophilic group on a linker reagent or drug. Useful nucleophilic groups on a linker reagent include, but are not limited to, hydrazide, oxime, amino, hydrazine, thiosemicarbazone, hydrazine carboxylate, and arylhydrazide. In one embodiment, an antibody is modified to introduce electrophilic moieties that are capable of reacting with nucleophilic substituents on the linker reagent or drug. In another embodiment, the sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody that can react with appropriate groups on the drug (Hermanson, Bioconjugate Techniques). In another embodiment, antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a drug moiety or linker nucleophile.

Nucleophilic groups on a drug moiety include, but are not limited to amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

The compounds of the invention expressly contemplate, but are not limited to, ADC prepared with the following cross-linker reagents: BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A.

Immunoconjugates comprising an antibody and a cytotoxic agent may also be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Alternatively, a fusion protein comprising an antibody and a cytotoxic agent may be made, e.g., by recombinant techniques or peptide synthesis. A recombinant DNA molecule may comprise regions encoding the antibody and cytotoxic portions of the conjugate either adjacent to one another or separated by a region encoding a linker peptide which does not destroy the desired properties of the conjugate.

In yet another embodiment, an antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Pharmaceutical Formulations

In one aspect, the invention further provides pharmaceutical formulations comprising at least one antibody of the invention and/or at least one immunoconjugate thereof. In some embodiments, a pharmaceutical formulation comprises 1) an antibody of the invention and/or an immunoconjugate thereof, and 2) a pharmaceutically acceptable carrier. In some embodiments, a pharmaceutical formulation comprises 1) an antibody of the invention and/or an immunoconjugate thereof, and optionally, 2) at least one additional therapeutic agent. Additional therapeutic agents include, but are not limited to, those described below.

Pharmaceutical formulations comprising an antibody or immunoconjugate of the invention are prepared for storage by mixing the antibody or immunoconjugate having the desired degree of purity with optional physiologically acceptable carriers, excipients or stabilizers (Remington's Pharmaceutical Sciences 16th edition, Osol, A. Ed. (1980)) in the form of aqueous solutions or lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride); phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Pharmaceutical formulations to be used for in vivo administration are generally sterile. This is readily accomplished by filtration through sterile filtration membranes.

Active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or immunoconjugate of the invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies or immunoconjugates remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

An antibody may be formulated in any suitable form for delivery to a target cell/tissue. For example, antibodies may be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA 82:3688 (1985); Hwang et al., Proc. Natl. Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257:286-288 (1982) via a disulfide interchange reaction. A chemotherapeutic agent is optionally contained within the liposome. See Gabizon et al., J. National Cancer Inst. 81(19):1484 (1989).

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include Zr⁸⁹, At²¹¹, I¹³¹, I¹²⁵, Y⁹⁰, Re¹⁸⁶, Re¹⁸⁸, Sm¹⁵³, Bi²¹², P³², P²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio)propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

Pharmaceutical Formulations

Pharmaceutical formulations of an anti-ETBR antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH2O, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide a BRAF inhibitor, a MEK inhibitor or an anti-CTLA-4 antibody, ipilimumab. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

Therapeutic Methods and Compositions

Any of the anti-ETBR antibodies provided herein may be used in therapeutic methods.

In one aspect, an anti-ETBR antibody for use as a medicament is provided. In another aspect, the methods provide for an anti-ETBR antibody in combination with a BRAF inhibitor as useful as a medicament. In further aspects, such a combination is useful in treating melanoma and/or metastatic melanoma. In certain embodiments, an anti-ETBR antibody in combination with a BRAF inhibitor for use in a method of treatment is provided. In certain embodiments, the invention provides an anti-ETBR antibody for use in a method of treating an individual having melanoma and/or metastatic melanoma comprising administering to the individual an effective amount of the anti-ETBR antibody and an effective amount of a BRAF inhibitor. In one such embodiment, the method further comprises administering to the individual an effective amount of at least one additional therapeutic agent, e.g., as described below, to the combination described. In further embodiments, the invention provides an anti-ETBR antibody in combination with a BRAF inhibitor for use in tumor growth inhibition (TGI). In certain embodiments, the invention provides an anti-ETBR antibody in combination with a BRAF inhibitor for use in a method of inhibiting tumor growth in a subject comprising administering to the subject an effective of the anti-ETBR antibody in combination with a BRAF inhibitor to inhibit tumor growth. A “subject” according to any of the above embodiments is preferably a human.

In a further aspect, the invention provides for the use of an anti-ETBR antibody in combination with a BRAF inhibitor in the manufacture or preparation of a medicament. In one embodiment, the medicament is for treatment of melanoma and/or metastatic melanoma. In a further embodiment, the medicament is for use in a method of treating melanoma and/or metastatic melanoma comprising administering to an individual having melanoma and/or metastatic melanoma an effective amount of the medicament. In one such embodiment, the method further comprises administering to the individual an effective amount of at least one additional therapeutic agent, e.g., as described below. In a further embodiment, the medicament is for tumor growth inhibition. In a further embodiment, the medicament is for use in a method of tumor growth inhibition in an individual comprising administering to the individual an amount effective of the medicament to inhibit tumor growth. An “individual” according to any of the above embodiments may be a human.

In a further aspect, the invention provides pharmaceutical formulations comprising any of the anti-ETBR antibodies provided herein, e.g., in combination with a BRAF inhibitor for use in any of the above therapeutic methods. In one embodiment, a pharmaceutical formulation comprises any of the anti-ETBR antibodies provided herein in combination with a BRAF inhibitor and a pharmaceutically acceptable carrier. In another embodiment, a pharmaceutical formulation comprises any of the anti-ETBR antibodies provided herein in combination with a BRAF inhibitor and at least one additional therapeutic agent, e.g., as described below.

Antibodies of the invention can be used either alone or in combination with other agents in a therapy. For instance, an antibody of the invention may be co-administered with at least one additional therapeutic agent. In certain non-limiting embodiments, an additional therapeutic agent is a BRAF inhibitor, a MEK inhibitor, or an anti-CTLA-4 antibody, such as, for example, ipilimumab.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent and/or adjuvant. Antibodies of the invention can also be used in combination with radiation therapy.

An antibody of the invention (and any additional therapeutic agent, such as, for example, a BRAF inhibitor) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. Alternatively, the BRAF inhibitor may be administered orally, in either tablet or capsule or liquid form.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE 2
SEQUENCES

| SEQ | | |
|---|---|---|
| ID NO: | Amino Acid Sequence | Structure |
| 1 | GYTFTSYWMQ | hu5E9.v1 and |
| | | hu5E9v.2 CDR- |
| | | H1 |
| 2 | TIYPGDGDTSYAQKFKG | hu5E9.v1 and |
| | | hu5E9v.2 CDR- |
| | | H2 |
| 3 | WGYAYDIDN | hu5E9.v1 and |
| | | hu5E9v.2 CDR- |
| | | H3 |
| 4 | KSSQSLLDSDGKTYLN | hu5E9.v1 and |
| | | hu5E9v.2 CDR- |
| | | L1 |
| 5 | LVSKLDS | hu5E9.v1 and |
| | | hu5E9v.2 CDR- |
| | | L2 |
| 6 | WQGTHFPYT | hu5E9.v1 and |
| | | hu5E9v.2 CDR- |
| | | L3 |
| 7 | EVQLVESGGGLVQPGGSLRLSCAASGYTFTSYWMQ | hu5E9.v1 |
| | WVRQAPGKGLEWIGTIYPGDGDTSYAQKFKGRATL | variable |
| | STDKSKNTAYLQMNSLRAEDTAVYY CARWGYAYDIDNWG | heavy chain |
| 8 | DIQMTQSPSSLSASVGDRVTITCKSSQSLLDSDGKTY | hu5E9.v1 and |
| | LNWLQQKPGKAPKRLIYLVSKLDSGVPSRFSGSG | hu5E9v.2 vari- |
| | SGTDFTLTISSLQPEDFATYYCWQGTHFPYTFGQGTKVEIK | able light |
| | | chains |
| 9 | EVQLVQSGAEVKKPGASVKVSCKASGYTFTSYWM | hu5E9.v2 |
| | QWVRQAPGQGLEWIGTIYPGDGDTSY | variable |
| | AQKFKGRVTITRDTSTSTAY | heavy chain |
| | LELSSLRSEDTAVYYCARWG | |
| | YAYDIDNWG | |
| 10 | MQPPPSLCGRALVALVLACGLSRIWGEERGFPPDRATPLL | Endothelin |
| | QTAEIMTPPTKTLWPKGSNASLARSLAPAEVPKGDRTAGS | receptor type B |
| | PPRTISPPPCQGPIEIKETFKYINTVVSCLVFVLGIIGNS | isoform 1 |
| | TLLRIIYKNKCMRNGPNILIASLALGDLLHIVIDIPINVY | variant |
| | KLLAEDWPFGAEMCKLVPFIQKASVGITVLSLCALSIDRY | [Homo sapiens] |
| | RAVASWSRIKGIGVPKWTAVEIVLIWVVSVVLAVPEAIGF | |
| | DIITMDYKGSYLRICLLHPVQKTAFMQFYKTAKDWWLFSF | |
| | YFCLPLAITAFFYTLMTCEM | |
| | LRKKSGMQIALNDHLKQRREVAKTVFCLVLVFALCWLPL | |
| | HLSRILKLTLYNQNDPNRCEL | |
| | LSFLLVLDYIGINMASLNSCINPIALYLVSKRFKNCFKSC | |
| | LCCWCQSFEEKQSLEEKQSCLKFKANDHGYDNFRSSNKYSSS | |

### EXAMPLES

The following are examples of methods and compositions of the invention. It is understood that various other embodiments may be practiced, given the general description provided above.

### Example 1

### In Vitro Evaluations of Specific Cell Killing by an Anti-ETBR ADC

The anti-ETBR antibody-ADC candidate Hu5E9v1-ADC was evaluated in vitro on melanoma cell lines expressing either relatively low ETBR copy number, in the case of cell line A2058 (Obtained from American Type Culture Collection) or high ETBR copy number, in the case of cell line UACC-257X2.2. The UACC-257X2.2 cell line is a derivative of the parental UACC-257 cell line (NCI-Frederick Cancer DCT Tumor Repository) optimized for growth in vivo. Parental UACC-257 cells were injected subcutaneously in the right flank of female NCr nude mice, one tumor was harvested and dissociated grown in vitro resulting in the UACC-257X1.2 cell line. The UACC-257X1.2 line was injected again subcutaneously in the right flank of female NCr nude mice in an effort to improve the growth of the cell line. A tumor from this study was collected and again adapted for in vitro growth to generate the UACC-257X2.2 cell line. This cell line expresses high levels of ET_{B}R as determined by flow cytometry. The relationship of receptor levels to Hu5E9v1-vc-MMAE cell killing in these cell lines was evaluated as follows.

The melanoma cell lines A2058 and UACC-257X2.2 were grown in appropriate media at 37° C. and 5% CO₂. To assess the effects of Hu5E9v1-ADC on cell viability, cells were plated at 1,500 per well in 50 μL, of normal growth medium in 96-well clear-bottom black plates. Twenty-four hours later, an additional 50 μL, of culture medium with serial dilutions of Hu5E9v1-ADC concentrations was added to triplicate wells. Five days later, cell survival was determined using CellTiter-Glo Luminescent Cell Viability Reagent (G7572; Promega Corporation) and with an EnVision 2101 Mutilabel Reader (Perkin-Elmer).

A determination of antibody binding sites per cell was performed (Scatchard analysis): The affinity constant and the number of cell surface binding sites for each antibody were estimated by incubating the melanoma cells for 4 h on ice with a fixed concentration of ¹²⁵I-labeled Hu5E9v1-ADC combined with increasing concentrations of unlabeled Hu5E9v1-ADC. The data were analyzed by nonlinear curve fitting using an analysis program method.

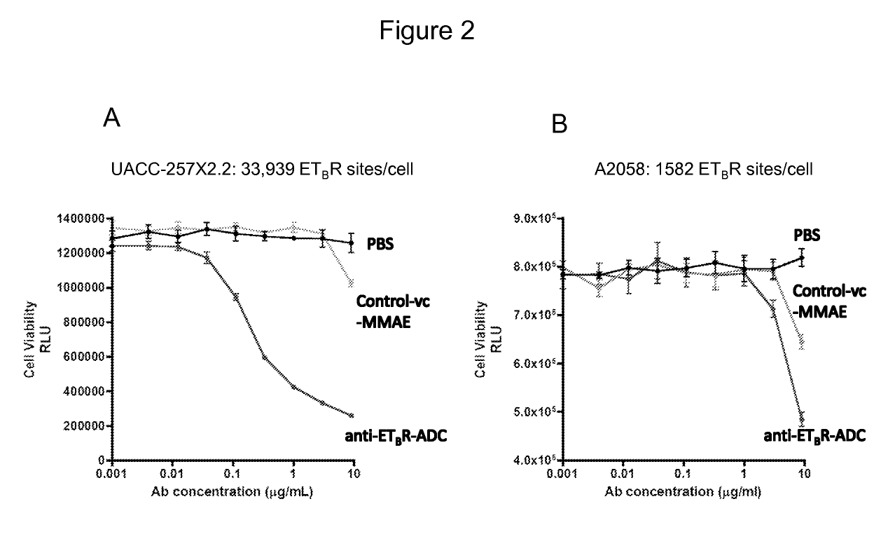

As shown in FIGS. 2A and 2B, by Scatchard analysis, the number of Hu5E9v1-ADC binding sites on A2058 and UACC-257X2.2 was estimated at 1,582 sites and 33,939 sites per cell, respectively. Titration of these cell lines with the anti-ETBR ADC candidate showed specific cell killing relative to control ADC that was generally proportional to the level of ETBR expression.

### Example 2

### In Vivo Evaluations of Specific Tumor Killing by an Anti-ETBR ADC

Based on the studies described in Example 1 above, melanoma cell lines A2058 and UACC-257X2.2 were selected as suitable models for in vivo anti-tumor activity studies that represent a wide range of ETBR expression. The UACC-257X2.2 melanoma cell line is a derivative of the parental UACC-257 melanoma cell line (National Cancer Institute (NCI)) optimized for growth in vivo. Specifically, parental UACC-257 cells were injected subcutaneously in the right flank of female NCr nude mice, one tumor was harvested and grown in vitro resulting in the UACC-257X1.2 cell line. The UACC-257X1.2 line was injected again subcutaneously in the right flank of female NCr nude mice in an effort to improve the growth of the cell line. A tumor from this study was collected and again adapted for in vitro growth to generate the UACC-257X2.2 cell line. This cell line and tumors derived from this line express ETBR comparable to the parental cell line UACC-257 (data not shown).

Next, efficacy studies were performed using the melanoma cells lines in the xenografts mouse models described above. All studies were conducted in accordance with the Guide for the Care and Use of Laboratory Animals (Ref: Institute of Laboratory Animal Resources (NIH publication no. 85-23), Washington, D.C.: National Academies Press; 1996). 10- to 14-week-old female CRL Nu/Nu or NCr nude mice from Charles River Laboratories were inoculated subcutaneously in the dorsal right flank with either 5×10⁶ UACC-257X2.2 cells in HBSS with Matrigel or 5×10⁶ A2058 cells in HBSS with Matrigel. When tumor volumes reached approximately 200 mm³ (day 0), animals were randomized into groups of 10 each.

For single agent efficacy studies, the anti-ETBR ADC candidate Hu5E9v1-ADC was administered as a single intravenous (IV) injection on day 0 at 1 mpk, 3 mpk, or 6 mpk (mg/kg). A control ADC antibody and vehicle control were also administered. Average tumor volumes with standard deviations were determined from 10 animals per group. Tumor volumes were measured twice per week until study end.

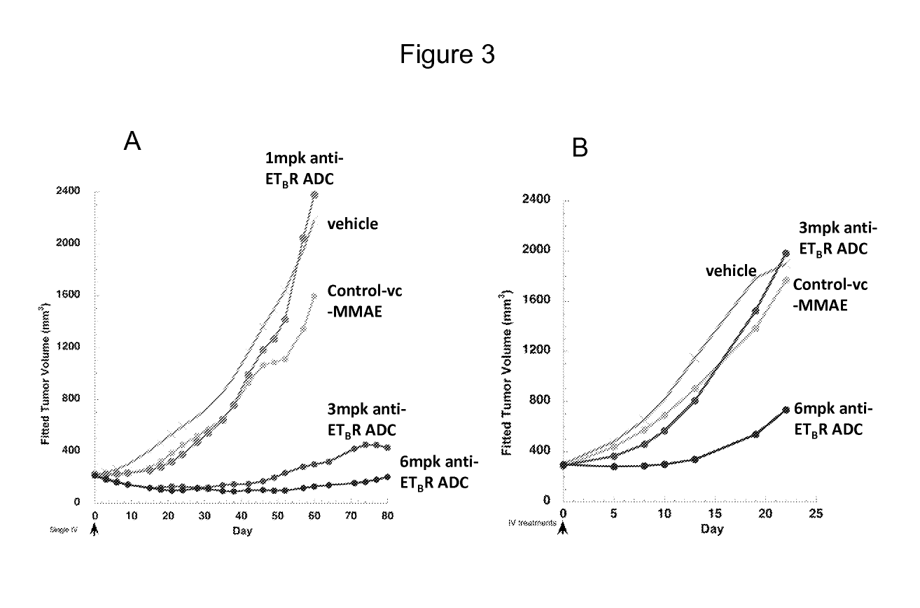

The results are shown in FIG. 3A for the high ETBR copy number UACC-257X2.2 cell line and 3B for the low ETBR copy number cell line A2058. Consistent with the in vitro cell killing experiments described in Example 1, the UACC-257X2.2 xenograft tumors were more responsive to the Hu5E9v1-ADC. While efficacy was not apparent for the group dosed at 1 mg/kg, sustained tumor regression was observed in response to single dose of 3 and 6 mg/kg Hu5E9v1-ADC (FIG. 3A).

Doses of 3 and 6 mg/kg of Hu5E9v1-ADC, 6 mg/kg control ADC or vehicle control were administered to animals bearing the low ETBR copy number A2058 tumors. A partial reduction in tumor burden was observed at the high dose of 6 mpk of the Hu5E9v1-ADC relative to the matching dose of control ADC or vehicle. Efficacy was not apparent for the group dosed at 3 mg/kg of Hu5E9v1-ADC. Thus, a reduction of tumor burden in the A2058 xenograft model that represents the low end of the spectrum of ETBR expression in human melanomas (Asundi et al, 2011) suggests that efficacy can be achieved with the candidate Hu5E9v1-ADC as a single agent in tumors that correspond to the full expression range of ETBR encountered in human melanomas.

### Example 3

### Effect of BRAF Inhibitor Drugs on the Expression Levels of ETBR

The effect of BRAF inhibitor drugs on the expression level of ETBR transcript and protein (total protein and cell surface protein) was evaluated in a variety of melanoma cells representing various genetic backgrounds of melanoma, such as mutant for BRAF(V600E), wild-type for BRAF and mutant for RAS (Q61L).

Melanoma cell lines UACC-257X2.2, A2058, COLO 829, IPC-298 (ATCC) were treated with a BRAF inhibitor drug (“BRAFi”), specifically RG7204 at varying concentrations by adding the appropriate drug volume to cells in culture for 24 h on four-well dishes.

To determine the effect of BRAFi on ETBR and control ribosomal protein L19 (RPL19) transcript levels, the following experiments were performed. Cells treated with RG7204 for 24 h were harvested from plates by scraping and processed for total RNA using Qiashredder and RNeasy mini kits (79654, 74104 from Qiagen, Valencia, Calif.). Taqman assays were set up using reagents from Applied Biosystems (ABI, Foster City, Calif.) and assayed using 7500 Real Time PCR machine and software from ABI. Primer-probe sets were designed with primers flanking a fluorogenic probe dual labeled with Reporter dye FAM and quencher dye TAMRA.

The primer-probe set for RPL19 is as follows:

(SEQ ID NO: 11)
Forward primer-5′ AGC GGA TTC TCA TGG AAC A;
(SEQ ID NO: 12)
Reverse primer-5′ CTG GTC AGC CAG GAG CTT
and
(SEQ ID NO: 13)
probe-5′ TCC ACA AGC TGA AGG CAG ACA AGG.

The primer-probe set for ETBR is as follows:

Forward primer-5′
(SEQ ID NO: 14)
TCA CTG AAT TCC TGC ATT AAC C,
reverse primer-5′
(SEQ ID NO: 15)
GCA TAA GCA TGA CTT AAA GCA GTT
and
probe-5′
(SEQ ID NO: 16)
AAT TGC TCT GTA TTT GGT GAG CAA AAG ATT CAA.

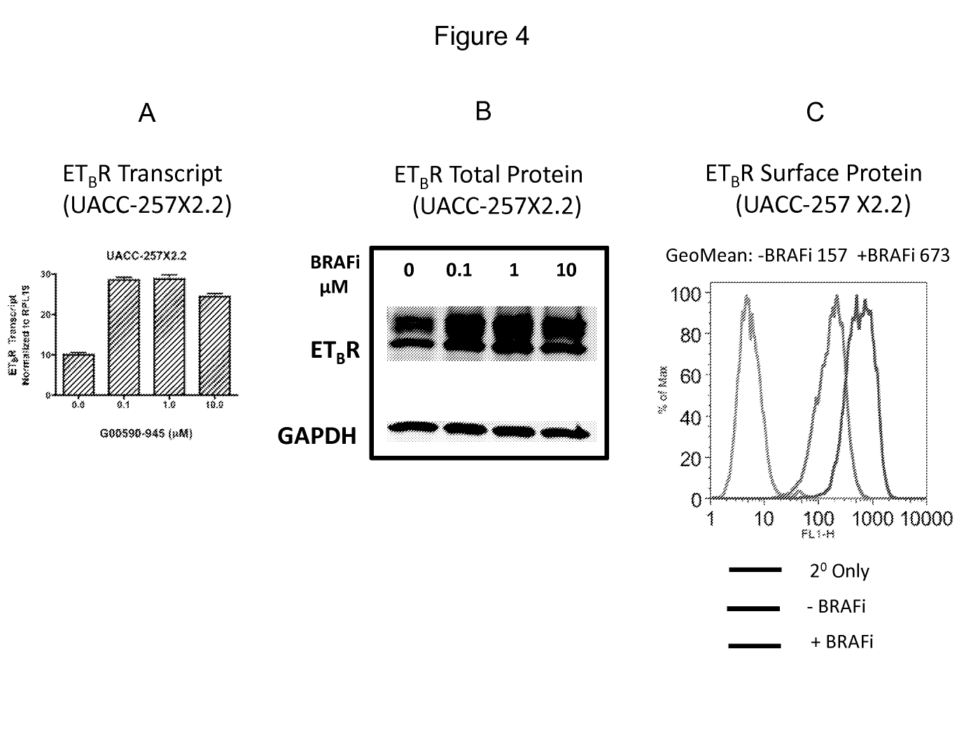

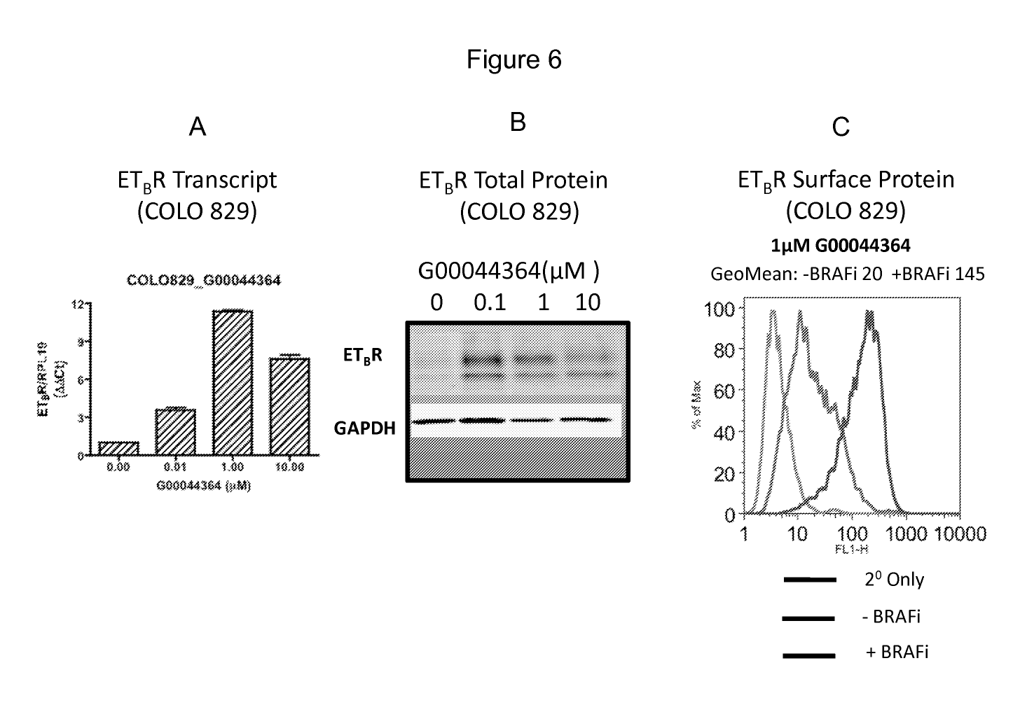

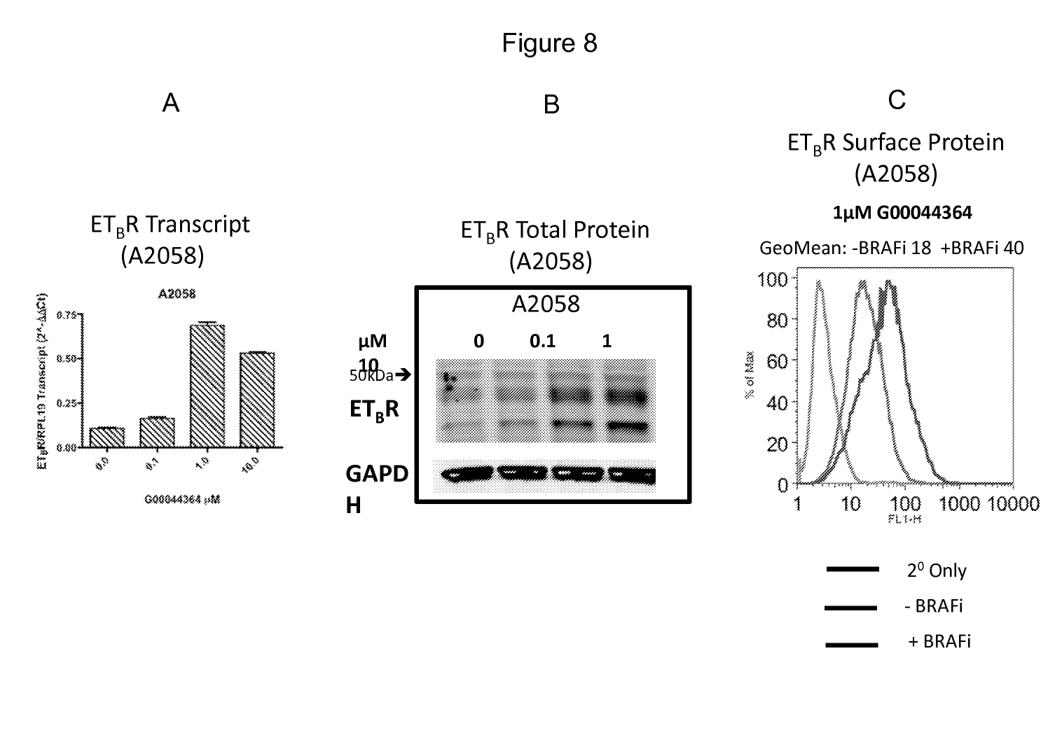

The results for UACC-257X2.2 are shown in FIG. 4A, the results for A2058 are shown in FIG. 8A, and the results for COLO 829 are shown in FIG. 6A. These results demonstrate that treatment with BRAFi RG7204 for 24 hours appears to increase the ETBR transcripts in all cell lines tested, as compared to control cells to which no BRAFi RG7204 was added.

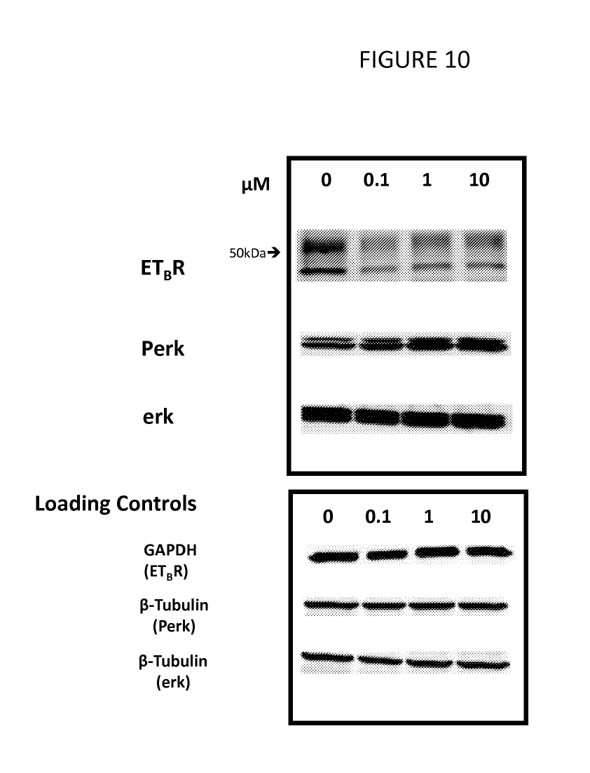

To test whether the increase in ETBR transcripts due to BRAFi treatment also results in any changes in ETBR total protein levels, Western blot experiments were performed on the same cell lines treated with BRAFi RG7204 as described above. For Western blotting, the following reagents were used: for detection of proteins: an anti-ETBR in-house generated monoclonal antibody 1H1.8.5, anti-Phospho-p44/42 MAPK (Erk1/2) (Thr202/Tyr204) antibody (9101, Cell Signaling Technology), anti-p44/42 MAPK (Erk1/2) antibody (9102, Cell Signaling Technology) and as controls, a rabbit polyclonal anti-GAPDH (glyceraldehyde-3-phosphate dehydrogenase) antibody (PA1-987; Affinity Bioreagents) and mouse monoclonal anti-β-Tubulin antibody (556321, BD Pharmingen). The results for UACC-257X2.2 are shown in FIG. 4B, the results for A2058 are shown in FIG. 8B, the results for COLO 829 are shown in FIG. 6B and the results for IPC-298 are shown in FIG. 10. These results demonstrate that treatment with BRAFi RG7204 for 24 hours appears to increase the ETBR total protein levels in the UACC-257X2.2, the A2058, and COLO 829 cell lines tested, as compared to control cells to which no BRAFi was added. However, with respect to the cell line IPC-298 which is wild-type for BRAF and mutant for RAS (Q61L), BRAFi does not appear to increase ETBR levels as compared over the various BRAFi dose levels, rather it appears to activate the levels of phosphor-ERK, as shown in FIG. 10.

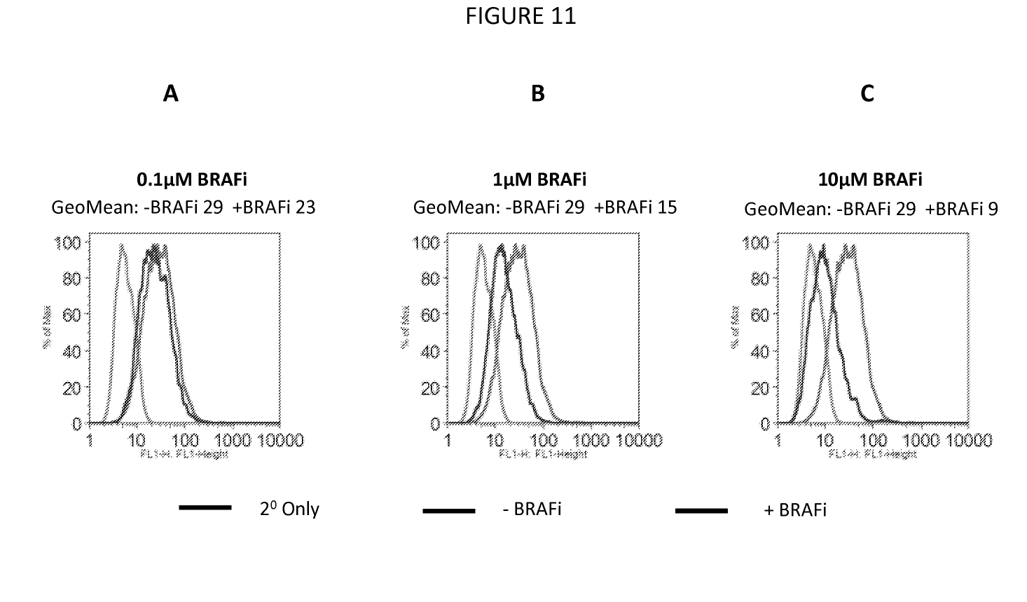

To determine whether the observed increases in total ET_{B}R protein level due to BRAFi treatment also results in an increase of ET_{B}R surface protein levels, a fluorescence-activated cell sorting (FACS) analysis was performed. Cells were harvested in PBS with 2.5 mmol/L EDTA and washed in PBS buffer containing 1% FBS. All subsequent steps were carried out at 4° C. Cells were incubated for 1 hour with 3 μg/mL anti ET_{B}R antibody Hu5E9v1, followed by anti-human IgG fluorescent detection reagent (A11013; Invitrogen). Cells were then analyzed with a FACS Calibur flow cytometer (BD Biosciences). The results for UACC-257X2.2 are shown in FIG. 4C, the results for A2058 are shown in FIG. 8C, and the results for COLO 829 are shown in FIG. 6C. These results demonstrate that treatment with BRAFi RG7204 for 24 h appears to increase the surface levels of ET_{B}R protein expressed in all cell lines tested, as compared to control cells to which no BRAFi was added. However, with respect to cell line IPC-298, BRAFi RG7204 appears to reduce ETBR levels at all dose levels tested, as shown in FIG. 11A-C.

### Example 4

### Effect of BRAF Inhibitor Drugs on In Vivo Efficacy of Anti-ETBR ADC

Given the results demonstrated in Example 3 above, the impact of the BRAF inhibitor drug on the in vivo efficacy of an anti-ET_{B}R ADC was tested. To do this, the in vivo efficacy for various combinations of Hu5E9v1-ADC and BRAFi-945 were evaluated against the UACC-257X2.2 melanoma model described above. Tumors were grown to an average size of approximately 200 mm³, whereupon animals were randomized into groups of 10 each. An appropriate vehicle control (Klucel LF) or BRAFi-945 at doses of 1 mpk, 6 mpk or 20 mpk were administered orally once a day×21 days beginning on study Day 0. A single 1 mpk or 3 mpk dose of Hu5E9v1-ADC or control, a histidine buffer #8, was administered intraveneously (after two doses of 945) via tail vein at study Day 1.

The average tumor volumes were determined from 10 animals per group. Tumor volumes were measured twice per week until study end. Tumor volumes were measured in two dimensions (length and width) using UltraCal IV calipers (Model 54 10 111, Fred V. Fowler Company; Newton, Mass.). The following formula was used with Excel, version 12.2.8 (Microsoft; Redmond, Wash.) to calculate tumor volume: Tumor Volume (mm3)=(length×width)×0.5

To analyze the repeated measurement of tumor volumes from the same animals over time, a mixed-modeling Linear Mixed Effects (LME) approach was used (Pinheiro et al. 2009). This approach can address both repeated measurements and a modest drop-out rate due to non-treatment-related termination of animals prior to study end. Cubic regression splines were used to fit a non-linear profile to the time courses of log 2 tumor volume at each dose level. These non-linear profiles were then related to dose within the mixed model. Tumor growth inhibition (TGI) as a percentage of vehicle was calculated as percent area under the fitted curve (AUC) per day in relation to the vehicle, using the following formula:

$$\% TGI = 100 \times [ 1 - ( \frac{{AUC}_{treatment} \text{/} day}{{AUC}_{vehicle} \text{/} day} ) ]$$

Using this formula, a TGI value of 100% indicates tumor stasis, of >1% but <100% indicates tumor growth delay, and of >100% indicates tumor regression. To get uncertainty intervals (UIs) for % TGI, the fitted curve and the fitted covariance matrix were used to generate a random sample as an approximation to the distribution of % TGI. The random sample is composed of 1000 simulated realizations of the fitted-mixed model, where the % TGI has been recalculated for each realization. Here, in the reported UI is the value for which 95% of the time, the recalculated values of % TGI will fall in this region given the fitted model. The 2.5 and 97.5 percentiles of the simulated distribution were used as the upper and lower UIs.

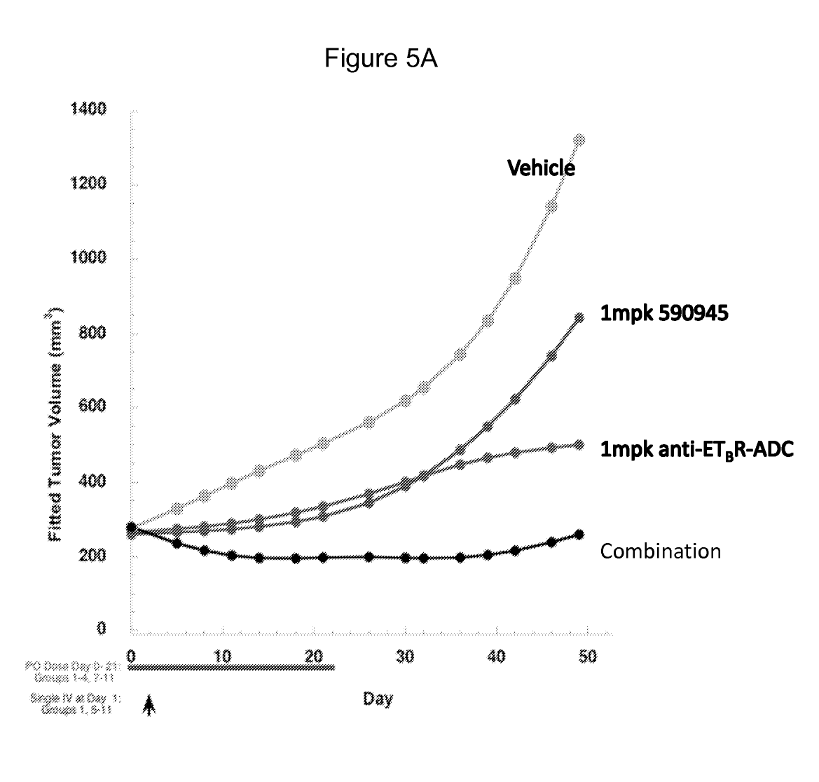

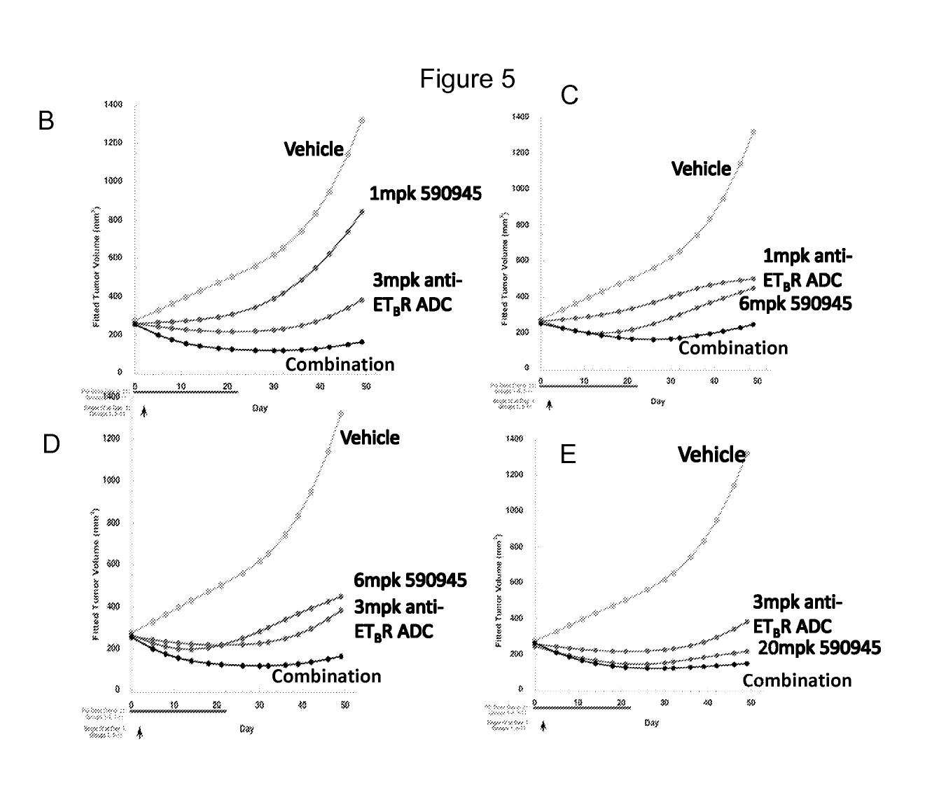

The results are shown in FIGS. 5A, 5B, 5C, 5D and 5E. All combinations of the Hu5E9v1-ADC and BRAFi-945 demonstrated better efficacy than either drug as a single agent alone. The two drugs combined at the lowest levels tested to give combination efficacy that was almost indistinguishable from the combination efficacy achieved at the highest dose levels tested.

### Example 5

### Dose Testing Anti-ETBR ADC and BRAFi Combinations In Vivo in COLO 829 Xenografts

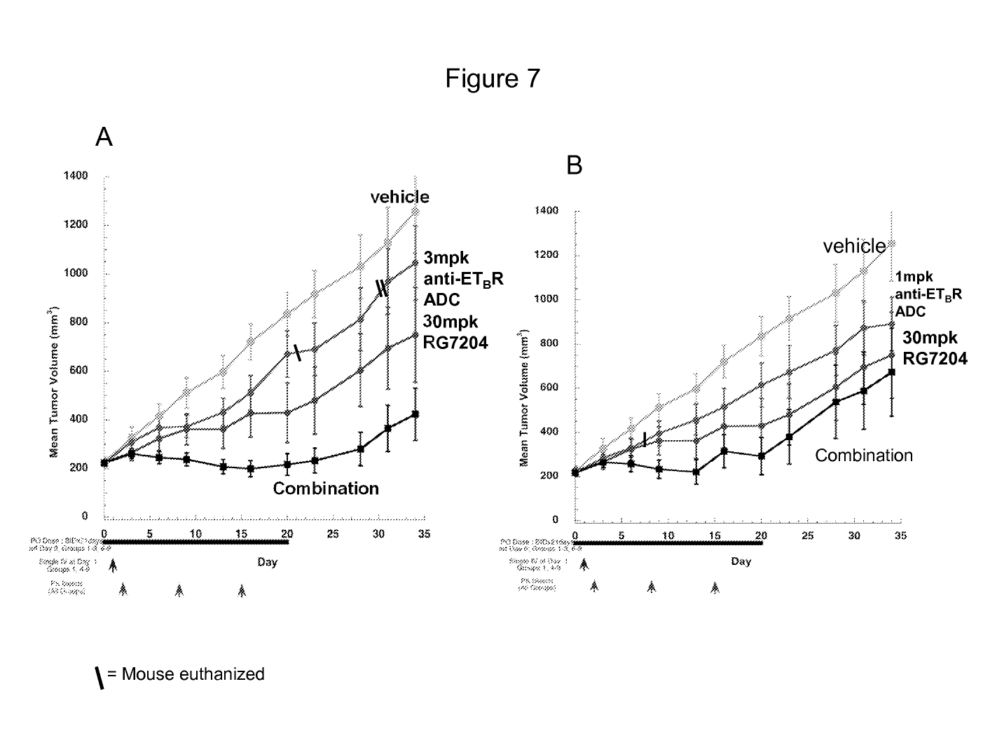

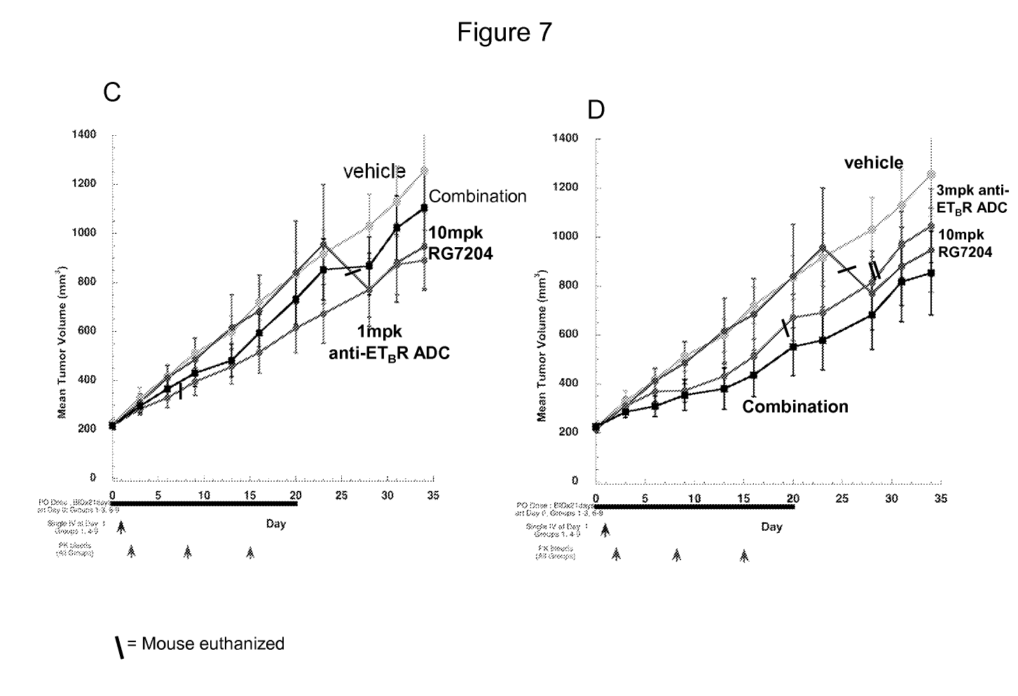

The study described in the example above allowed a refinement of the evaluation of in vivo combination efficacy of Hu5E9v1-ADC with the BRAF inhibitor drug RG7204. A lack of antagonism between the drugs was anticipated, thus the combination efficacy of the drugs were tested at lower doses. The COLO 829 xenograft model was chosen as representative of medium levels of ET_{B}R expression, further increasing the stringency of the combination studies. Tumors were grown to an average size of approximately 200 mm³, whereupon animals were randomized into groups of 9 each. An appropriate vehicle control (Klucel LF) or G00044364.1-12 (RG7204) at doses of 10 mpk or 30 mpk were administered orally twice a day for 21 days starting on day 0. A single dose of Hu5E9v1-ADC at either 1 mpk or 3 mpk or control, a histidine buffer #8, was administered intravenously on day 1 (after three doses of RG7204). The results are shown in FIGS. 7A, 7B, 7C and 7D.

Mid range doses of both drugs (30 mg/kg RG7204 and 3 mg/kg of Hu5E9v1-ADC) combined well together to give combination efficacy greater than either drug alone. Other combinations of the two drugs at lower doses trended similarly, with the sole exception of the lowest doses tested in combination.

### Example 6

### Dose Testing Anti-ETBR ADC and BRAFi Combinations In Vivo in A2058 Xenografts

The efficacy of Hu5E9v1-ADC and RG7204 in the A2058 xenograft model was tested. This model is of particular interest due to the high level of stringency it represents. The A2058 xenograft model represents the lower end of the ETBR expression spectrum found in melanoma patients, thereby making it a challenging model for achieving anti-ETBR ADC efficacy. Further, in spite of its BRAF V600E mutational status, this model has been demonstrated to be non-responsive to RG7204 with an in vitro killing efficacy of >20 μM (data not shown).

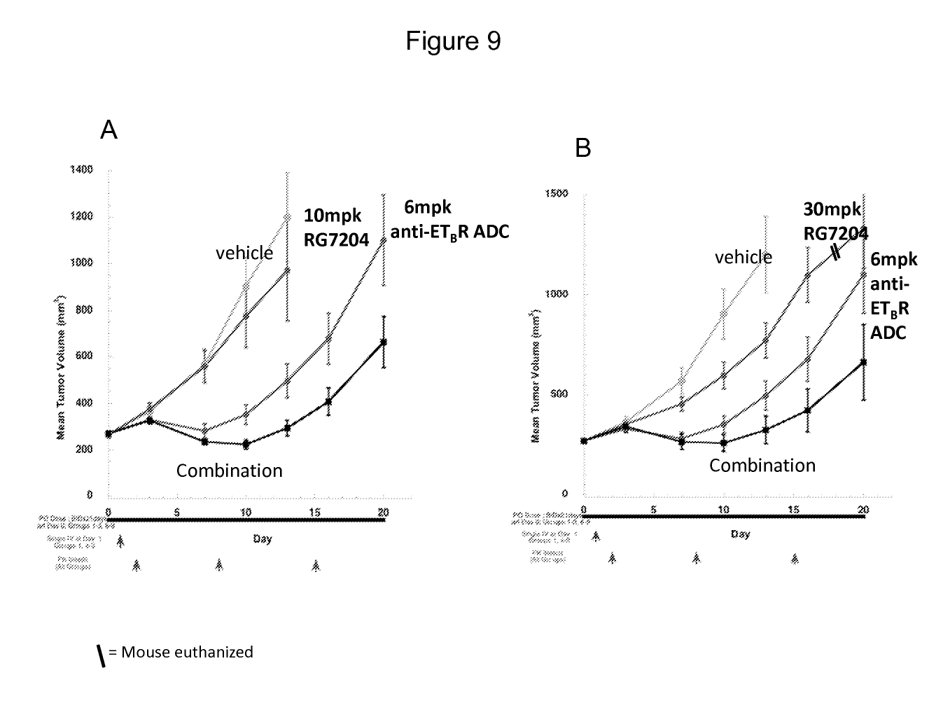

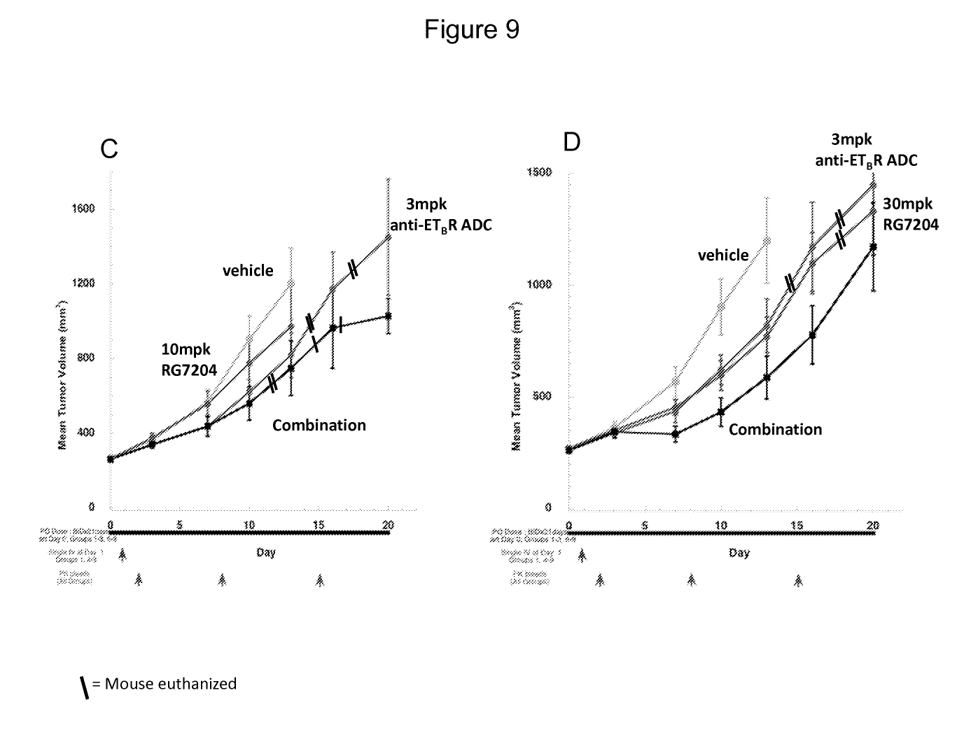

Tumors were grown to an average size of approximately 200 mm³, whereupon animals were randomized into groups of 10 each. An appropriate vehicle control (Klucel LF) or RG7204 at doses of 10 mpk or 30 mpk were administered orally twice a day for 21 days starting on day 0. A single dose of Hu5E9v1-ADC at either 3 mpk or 6 mpk or control, a histidine buffer #8, was administered intraveneously into the tail vein on day 1 (after three doses of RG7204). The results are shown in FIGS. 9A, 9B, 9C, and 9D.

The results show that in all cases tested, the combination of Hu5E9v1-ADC and RG7204 demonstrated greater efficacy than any single agent alone. A 10 mg/kg dose of RG7204 alone did not show single agent efficacy against the A2058 model (see FIGS. 9A and 9C). However, when combined with a 6 mg/kg dose of Hu5E9v1-ADC, a better efficacy was achieved than with either agent alone (see FIGS. 9A and 9C). The combination of 10 mg/kg RG7204 with 6 mg/kg of Hu5E9v1-ADC (FIG. 9A) demonstrated combination efficacy almost indistinguishable from the combination efficacy achieved at the highest dose levels tested, i.e., 30 mpk RG7204 and 6 mpk Hu5E9v1-ADC as shown in FIG. 9B.

Table 3 summarizes the three melanoma xenograft models tested at varying doses, as described above, to demonstrate the combination effects, expressed as a percent delta (last column) of the combination use of anti-ETBR ADC with a BRAF inhibitor as compared to either the percent TGI of the anti-ETBR ADC as a single agent or the percent TGI of a BRAF inhibitor as a single agent. The percent TGI was calculated using a Linear Mixed Effects (LME) modeling approach, as described above.

### Example 7

### Effect of MEK Inhibitor Drugs on the Expression Levels of ETBR

The effect of MEK inhibitor drugs on the expression level of ETBR transcript and protein (total protein and cell surface protein) was evaluated in a variety of melanoma cells that are either BRAF wild-type or mutational and/or RAS wild type or mutational: COLO829 (BRAF^{V600E}), A2058 (BRAF^{V600E}), SK23-MEL (BRAF^{WT}/RAS^{WT}), or IPC-298 (BRAF^{WT}/RAS^{C61L}).

Melanoma cell lines A2058, COLO 829, SK23-MEL and IPC-298 (ATCC) were treated with a MEK inhibitor drug (“MEKi-973” or “MEKi-623”), at varying concentrations (0 μM, 0.01 μM, 0.1 μM or 1 μM) by adding the appropriate drug volume to cells in culture for 24 h on four-well dishes.

To determine the effect of MEKi on ETBR transcript levels, the following experiments were performed as described above in Example 3. Cells treated with either MEKi-973 for 24 h were harvested from plates by scraping and processed for total RNA using Qiashredder and RNeasy mini kits (79654, 74104 from Qiagen, Valencia, Calif.). Taqman assays were set up using reagents from Applied Biosystems (ABI, Foster City, Calif.) and assayed using 7500 Real Time PCR machine and software from ABI. Primer-probe sets were designed with primers flanking a fluorogenic probe dual labeled with Reporter dye FAM and quencher dye TAMRA.

The primer-probe set for RPL19 is as follows:

(SEQ ID NO: 11)
Forward primer-5′ AGC GGA TTC TCA TGG AAC A;
(SEQ ID NO: 12)
Reverse primer-5′ CTG GTC AGC CAG GAG CTT
and
(SEQ ID NO: 13)
probe-5′ TCC ACA AGC TGA AGG CAG ACA AGG.

The primer-probe set for ETBR is as follows:

Forward primer-5′
(SEQ ID NO: 14)
TCA CTG AAT TCC TGC ATT AAC C,
reverse primer-5′
(SEQ ID NO: 15)
GCA TAA GCA TGA CTT AAA GCA GTT
and
probe-5′
(SEQ ID NO: 16)
AAT TGC TCT GTA TTT GGT GAG CAA AAG ATT CAA.

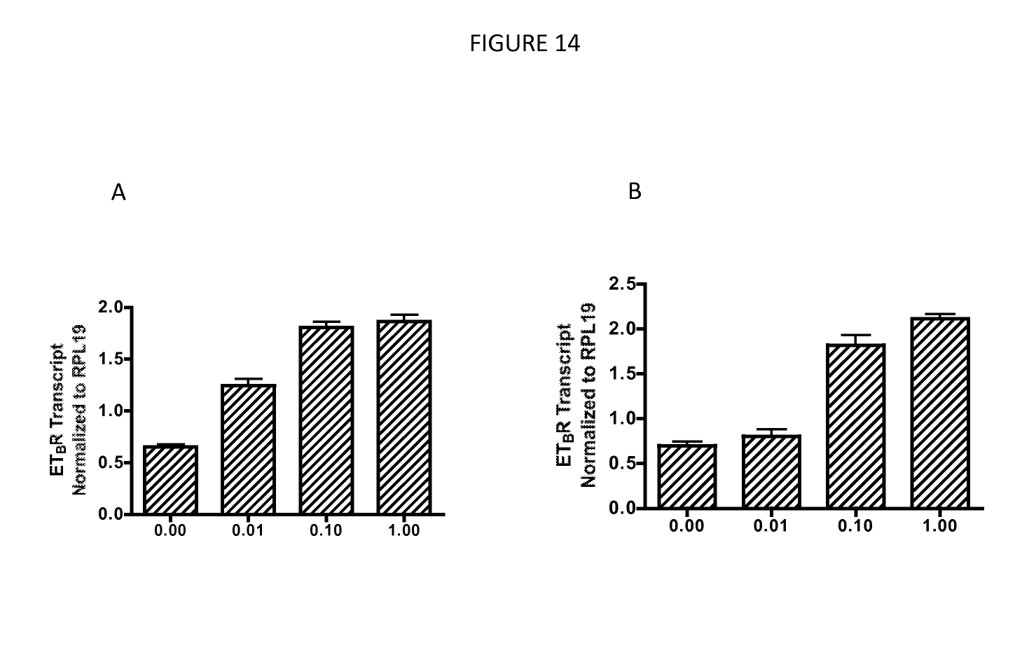

The results for A2058 are shown in FIG. 14A treated with MEKi-623 and 14B treated with MEKi-973 at the indicated doses. These results demonstrate that treatment with a MEK inhibitor for 24 hours appears to increase the ETBR transcripts, as compared to control cells to which no MEK inhibitor was added.

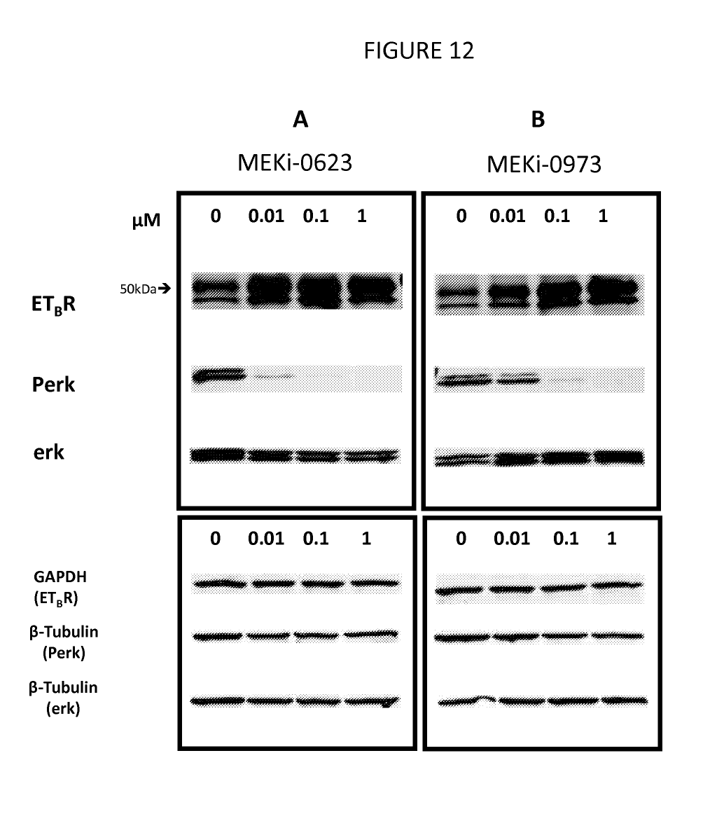

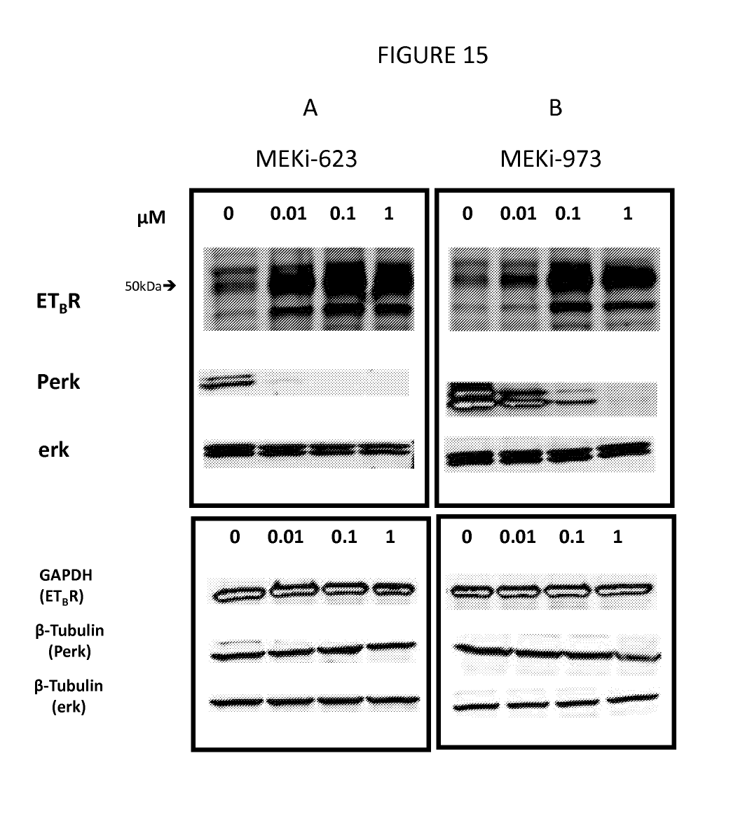

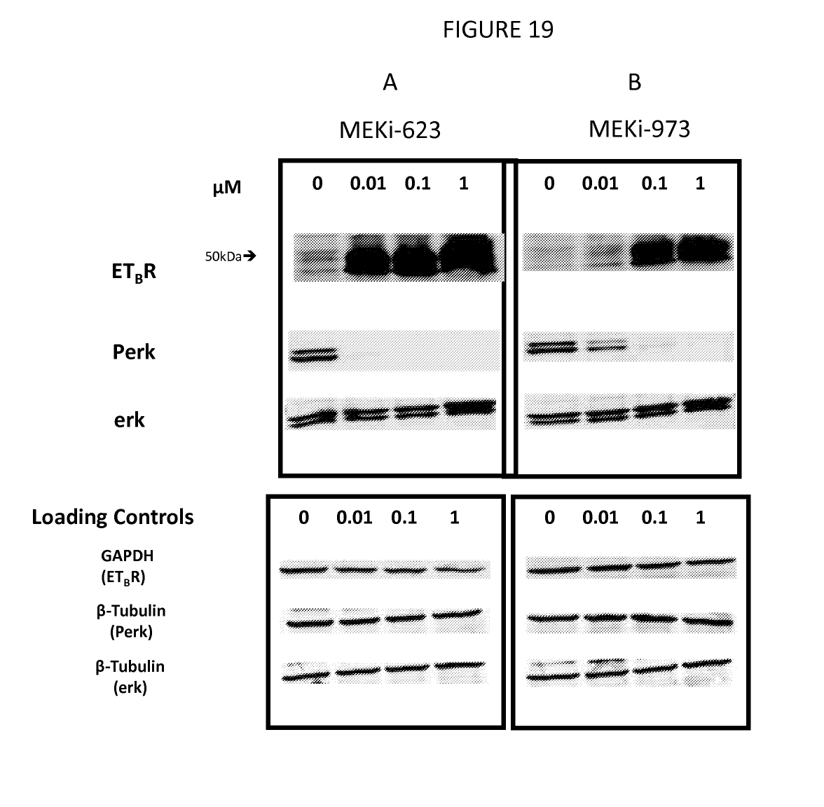

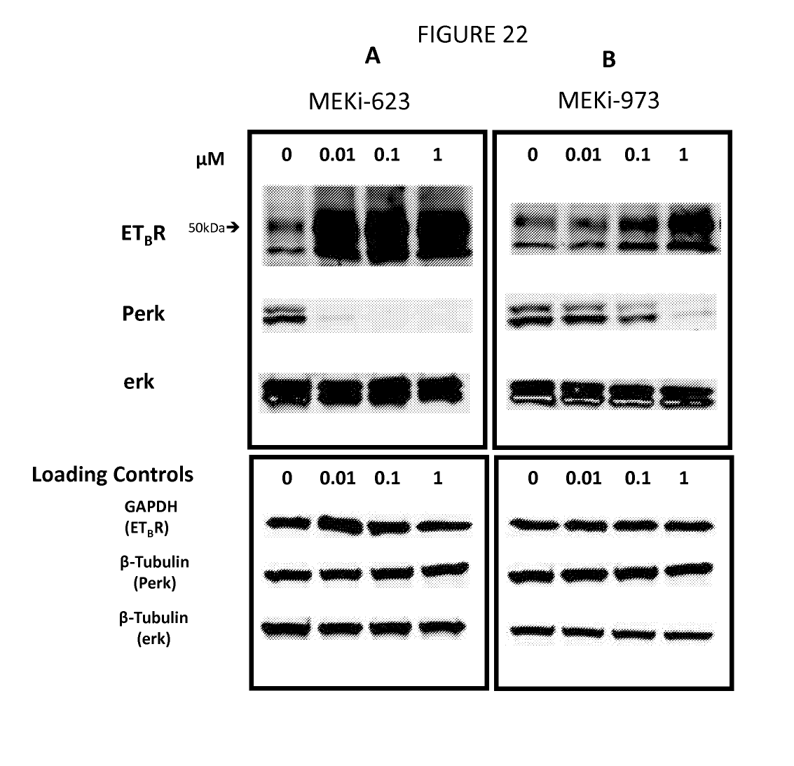

To test whether the increase in ETBR transcripts due to MEKi treatment also results in any changes in ETBR total protein levels, Western blot experiments were performed on cell lines COLO829 (BRAF^{V600E}), A2058 (BRAF^{V600E}), SK23-MEL (BRAF^{WT}/RAS^{WT}), or IPC-298 (BRAF^{WT}/RAS^{C61L}) which were treated with MEKi-973 as described above. For Western blotting, the following reagents were used: for detection of proteins: an anti-ETBR in-house generated monoclonal antibody 1H1.8.5, anti-Phospho-p44/42 MAPK (Erk1/2) (Thr202/Tyr204) antibody (9101, Cell Signaling Technology), anti-p44/42 MAPK (Erk1/2) antibody (9102, Cell Signaling Technology) and as controls, a rabbit polyclonal anti-GAPDH (glyceraldehyde-3-phosphate dehydrogenase) antibody (PA1-987; Affinity Bioreagents) and mouse monoclonal anti-β-Tubulin antibody (556321, BD Pharmingen). The results for A2058 are shown in FIG. 15A treated with MEKi-623 and 15B treated with MEKi-973, the results for COLO 829 are shown in FIG. 12A treated with MEKi-623 and 12B treated with MEKi-973, the results for SK23-MEL are shown in FIG. 19A treated with MEKi-623 and 19B treated with MEKi-973, and the results for IPC-298 are shown in FIG. 22A treated with MEKi-623 and 22B treated with MEKi-973. These results demonstrate that treatment with MEKi-623 or MEKi-973 for 24 hours appears to increase the ETBR total protein levels in all the cell lines tested, as compared to control cells to which no MEKi was added.

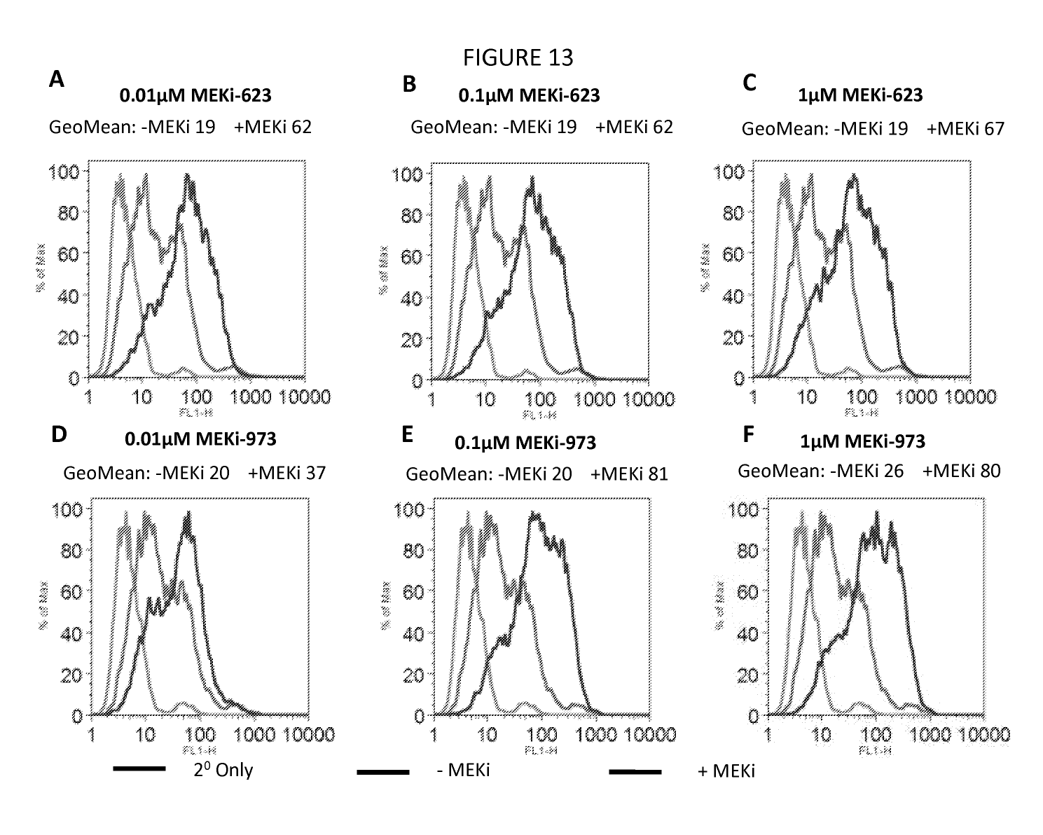

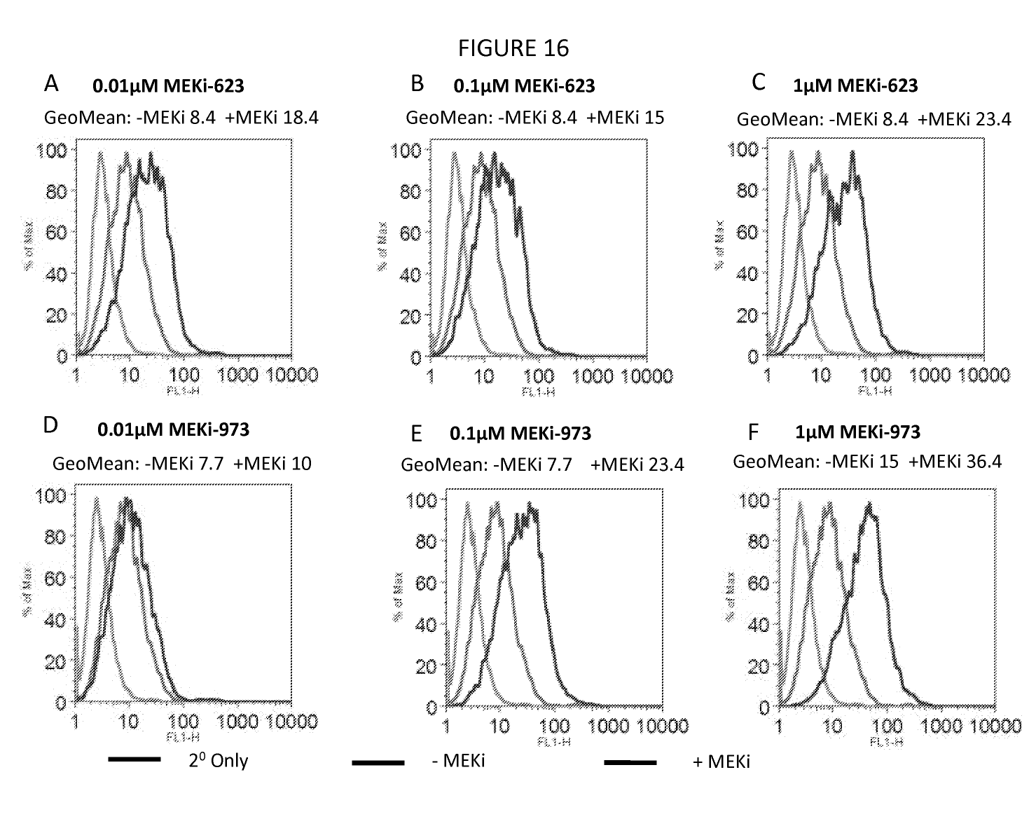

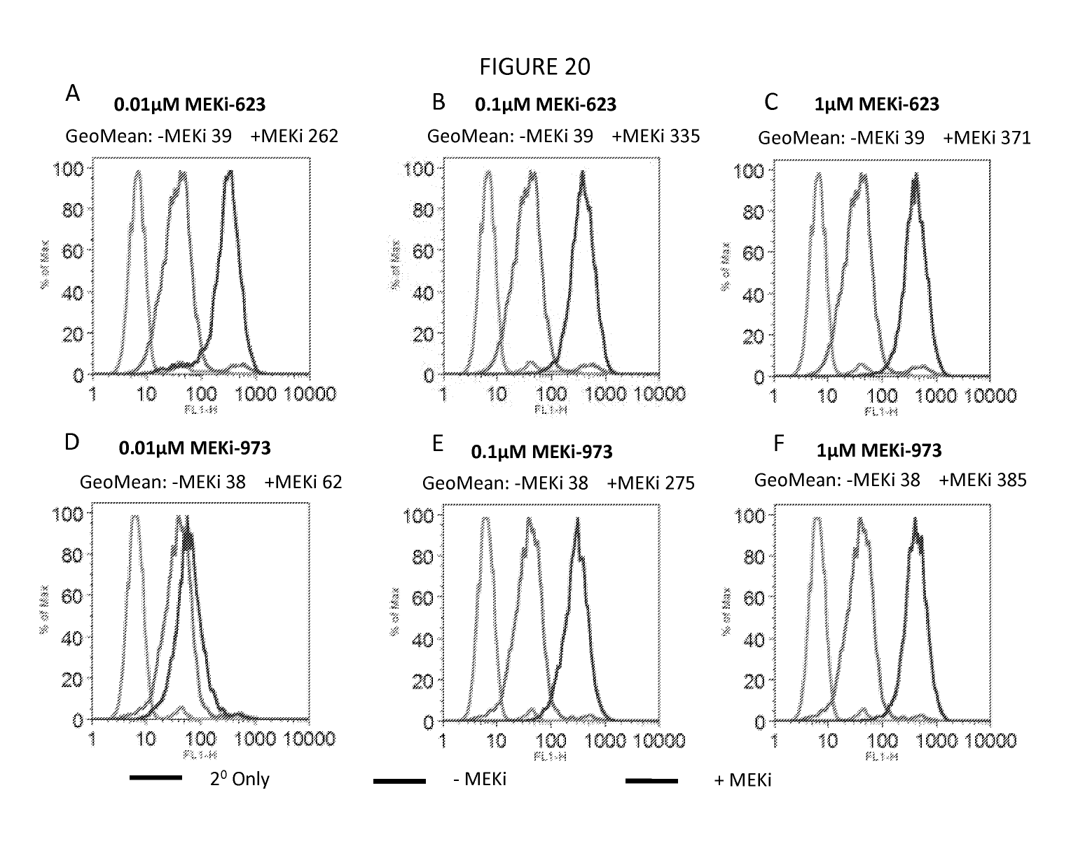

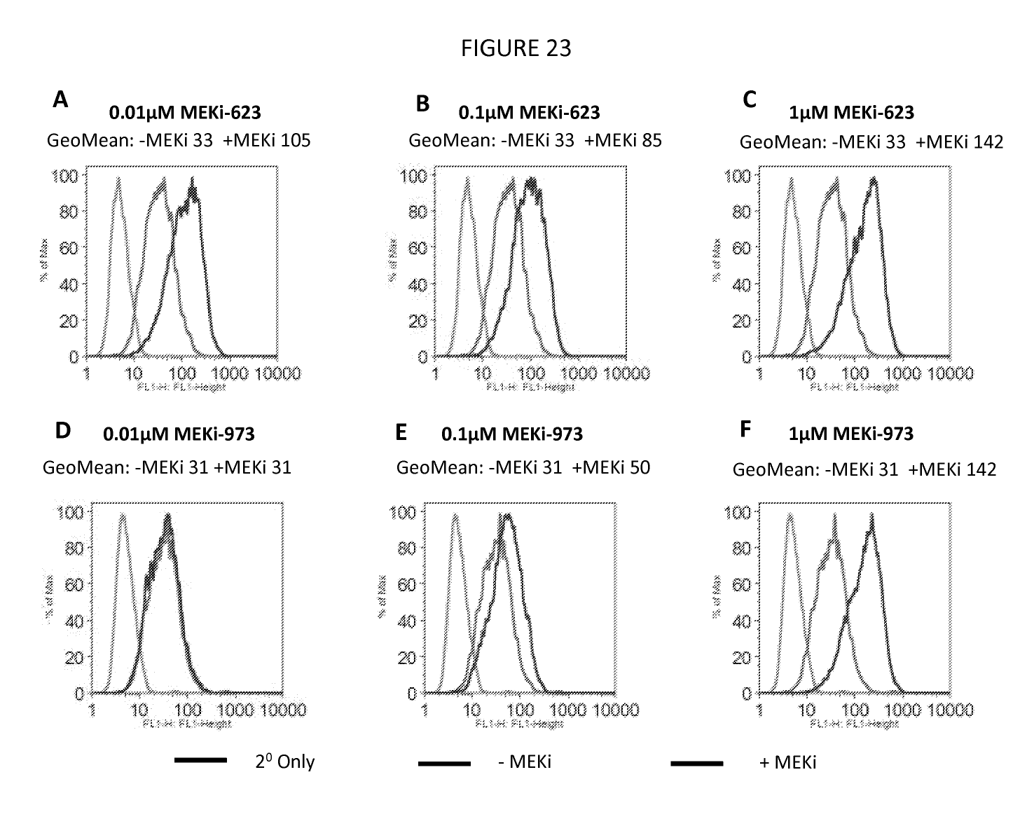

To determine whether the observed increases in total ET_{B}R protein level due to MEKi treatment also results in an increase of ET_{B}R surface protein levels, a fluorescence-activated cell sorting (FACS) analysis was performed as described above. Cells were harvested in PBS with 2.5 mmol/L EDTA and washed in PBS buffer containing 1% FBS. All subsequent steps were carried out at 4° C. Cells were incubated for 1 hour with 3 μg/mL anti ET_{B}R antibody Hu5E9v1, followed by anti-human IgG fluorescent detection reagent (A11013; Invitrogen). Cells were then analyzed with a FACS Calibur flow cytometer (BD Biosciences). The results for A2058 are shown in FIG. 16A-F, results for COLO829 are shown in FIG. 13A-F, the results for SK23-MEL are shown in FIG. 20A-F and the results for IPC-298 are shown in FIG. 23A-F. These results demonstrate that treatment with MEKi-623 or MEKi-973 for 24 h appears to increase the surface levels of ET_{B}R protein expressed in all cell lines tested, as compared to control cells to which no MEK inhibitor was added.

### Example 8

### Effect of MEK Inhibitor Drugs on In Vivo Efficacy of Anti-ETBR ADC

Given the results demonstrated in Example 7 above, the impact of the MEK inhibitors described herein on the in vivo efficacy of an anti-ET_{B}R ADC was tested. To do this, the in vivo efficacy for various combinations of Hu5E9v1-ADC and MEKi-623 and/or MEKi-973 were evaluated against A2058 and SK-MEL-23 and IPC-298 melanoma in vivo models, performed as described above in Example 4. An appropriate methylcellulose tween vehicle control (0.5% methylcellulose, 0.2% Tween-80 (MCT) or MEK inhibitor at doses of 1 mpk, 3 mpk or 7.5 mpk were administered orally once a day×21 days beginning on study Day 0. A single 3 mpk or 6 mpk dose of Hu5E9v1-ADC or control, a histidine buffer #8, was administered intraveneously (after two doses of a MEK inhibitor) via tail vein at study Day 1.

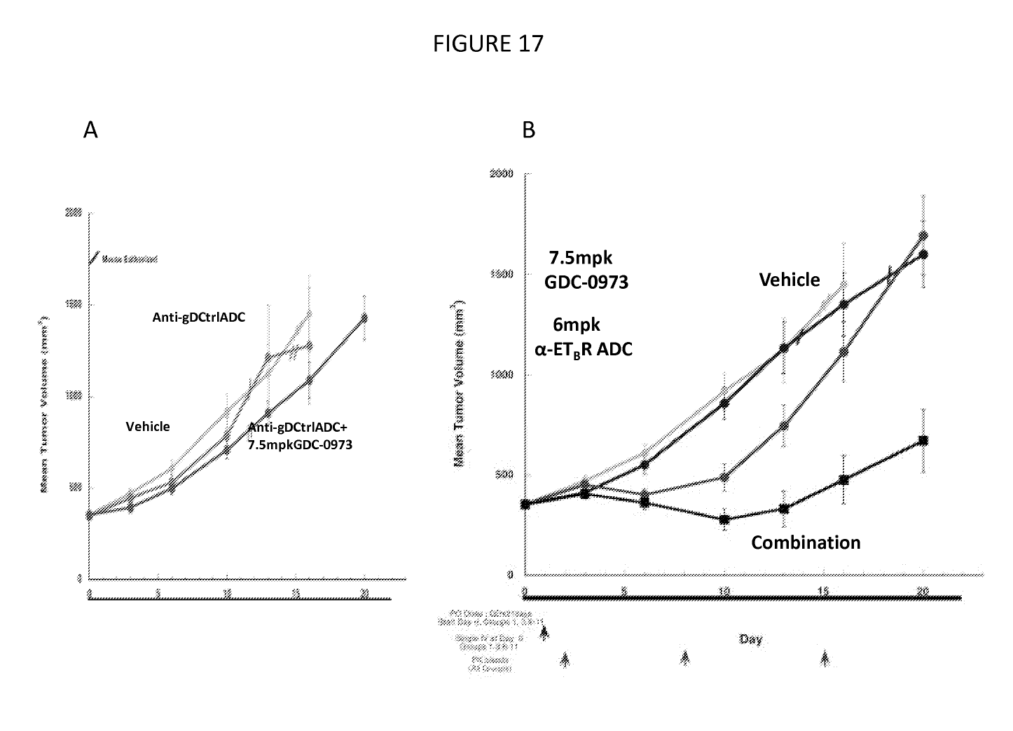

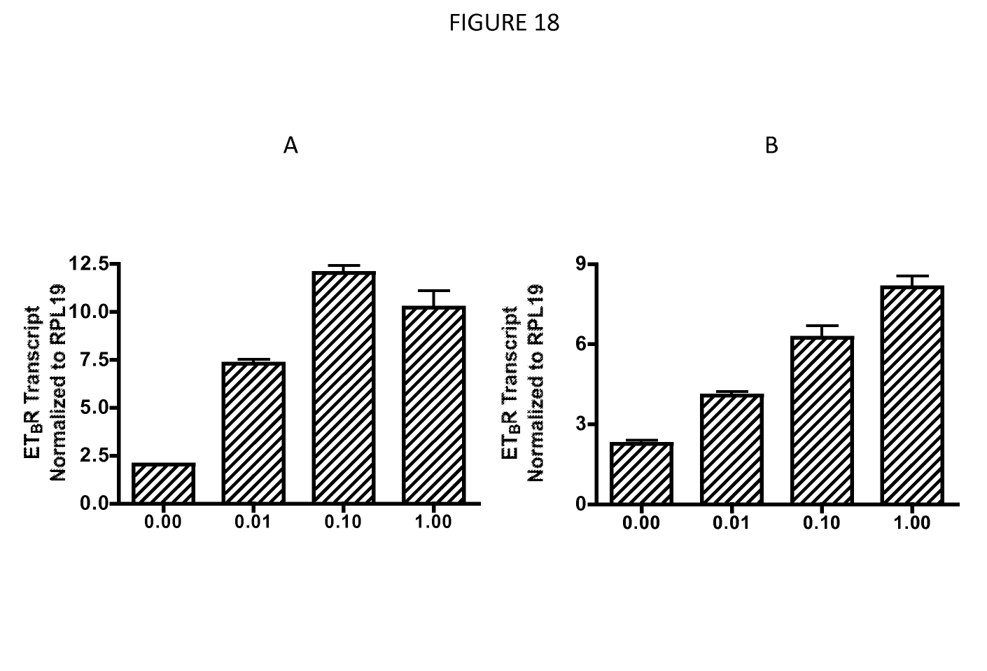

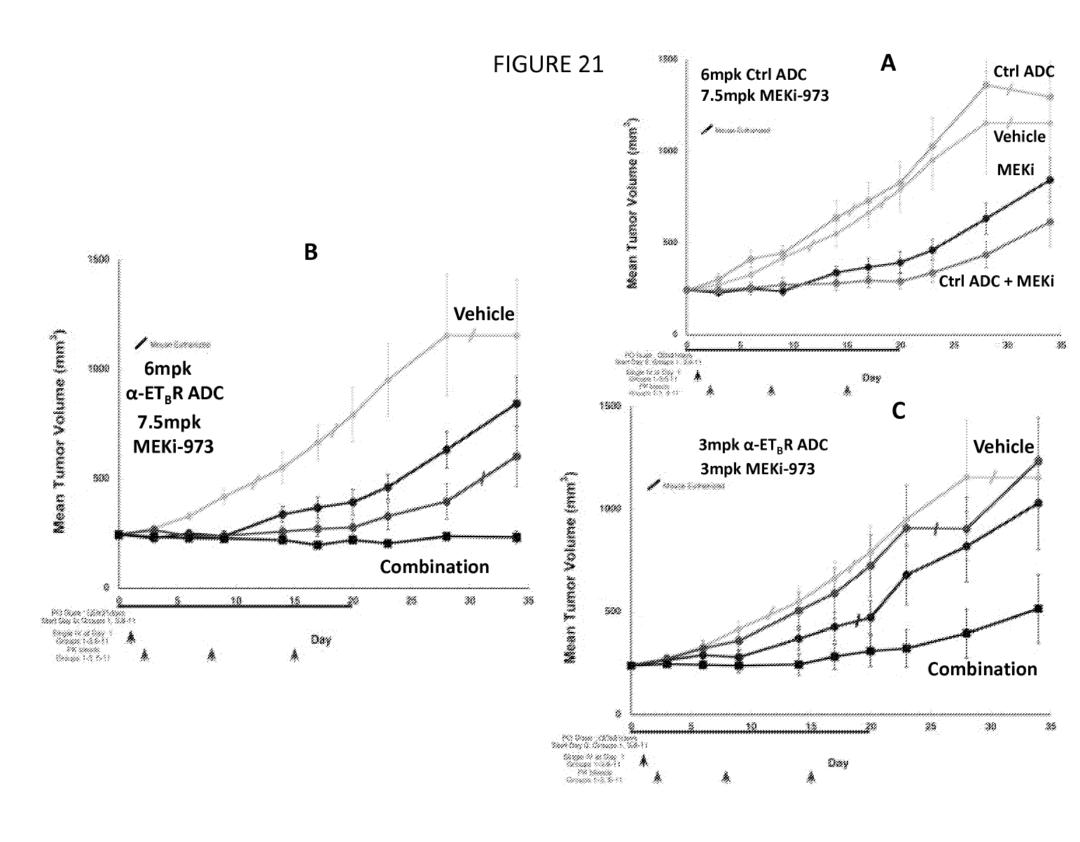

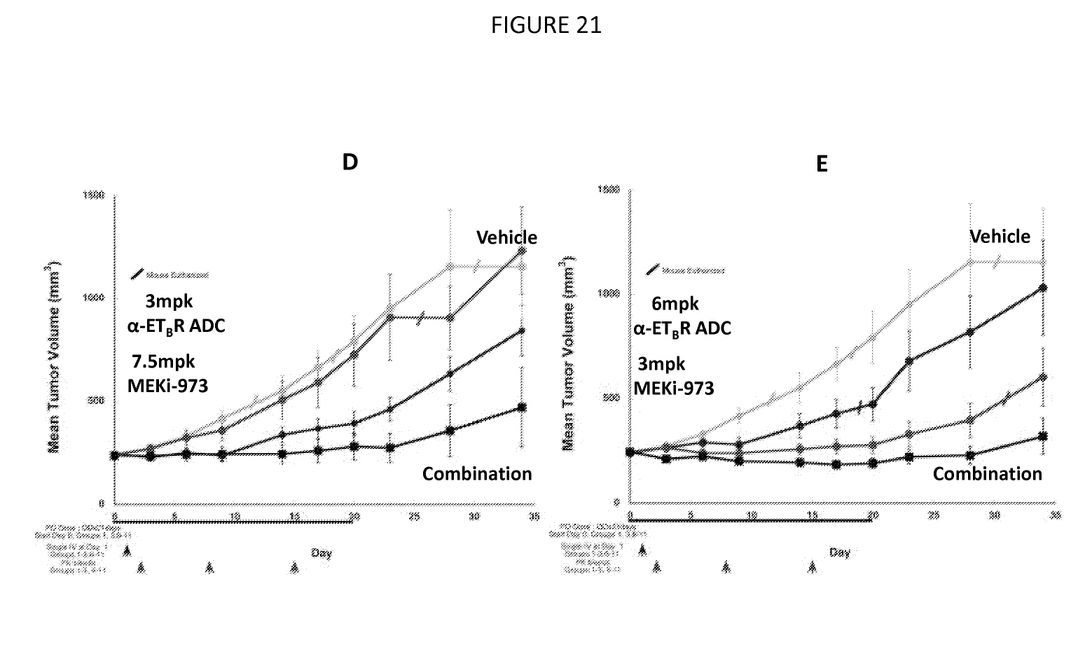

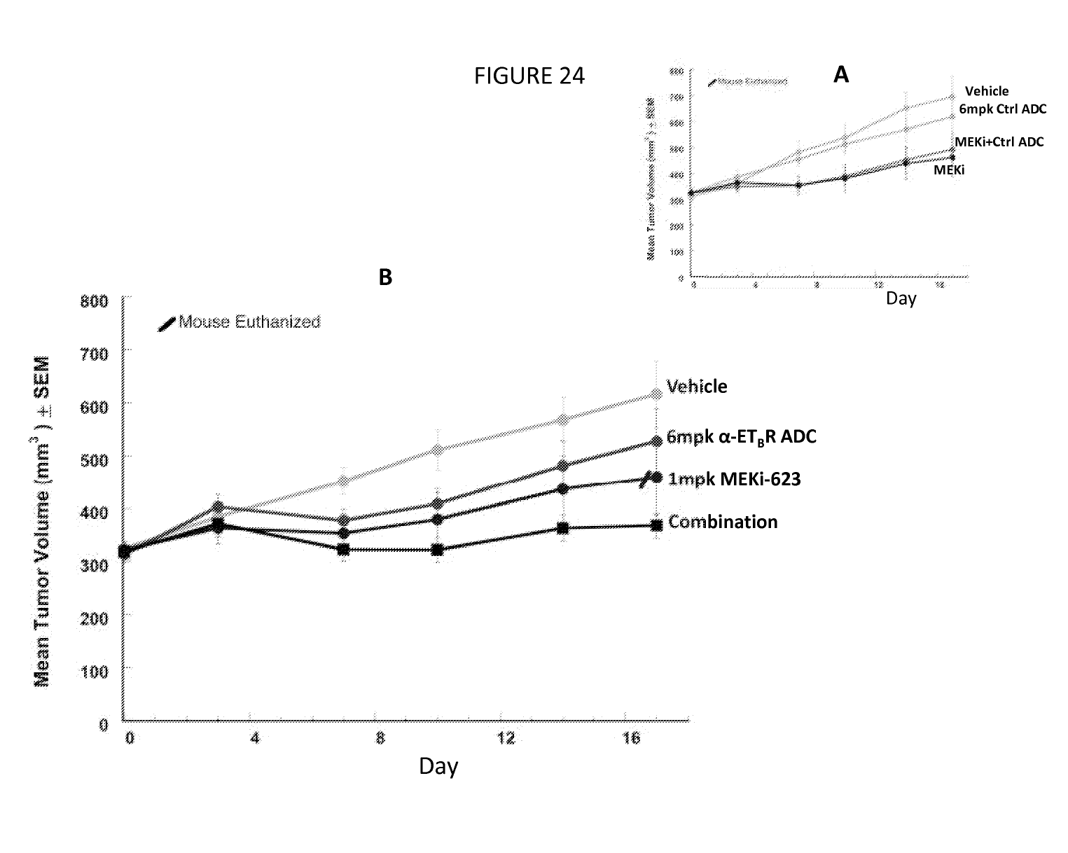

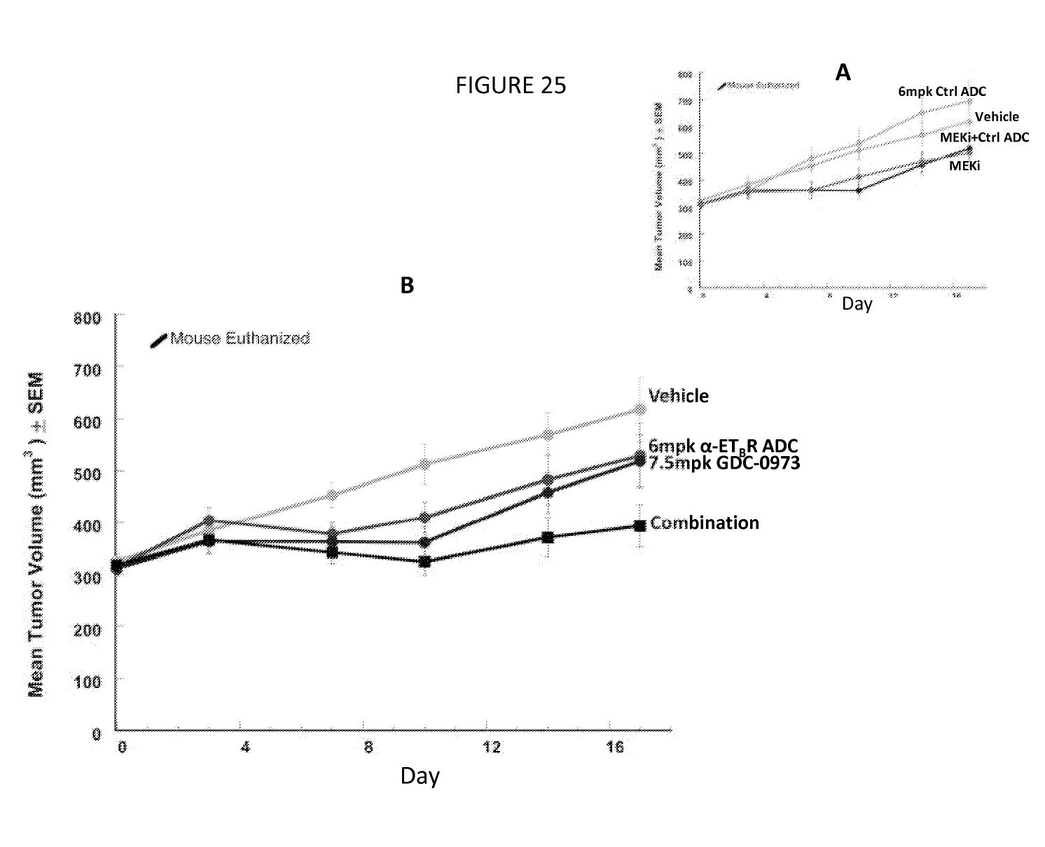

The results are shown in FIGS. 17A-B, FIG. 21, FIG. 24 and FIG. 25. Surprisingly, all combinations of the Hu5E9v1-ADC and MEK inhibitors tested demonstrated efficacy greater than the additive efficacy of either drug as a single agent alone.

### Example 9

### PD Studies of A2058 and COLO 829 Melanoma Xenografts

Tumors collected at the end of studies represented in FIG. 7 (COLO 829 vs combination anti-ETBR-ADC and BRAFi RG7204) did not show an increase of ETBR. This could be due to the fact that the timing of the tumor collection (day 34) was well past the wash out period of the BRAFi drug administered. In order to evaluate whether the in vitro effects of BRAFi/MEKi on cell lines, (i.e. increase of ETBR and decrease of Perk), also occurs in vivo, and therefore allows for a greater efficacy of anti-ETBR ADC and BRAFi/MEKi in combination, the following experiments were performed.

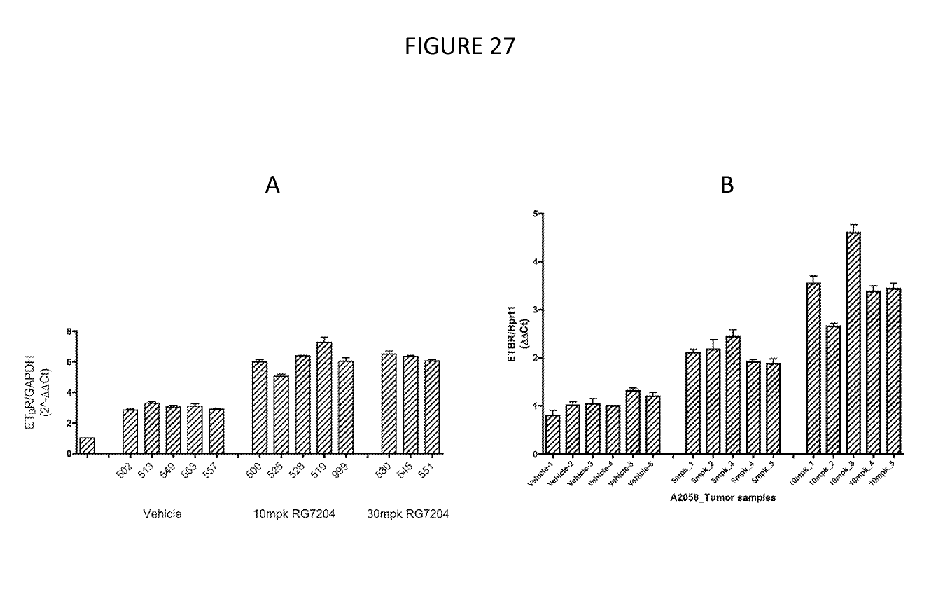

A2058 or COLO 829 tumors were grown to an average size of approximately 200 mm³, whereupon animals were randomized into groups of 5-6 each. For the BRAFi PD study, an appropriate vehicle control (Klucel LF) or RG7204 at doses of 10 mpk or 30 mpk were administered twice a day for 3 days (FIG. 27A). For the MEKi PD study, an appropriate vehicle control or MEKi-973 at doses of 5 mpk and 10 mpk were administered orally once a day for 3 days (FIG. 27B). Flash frozen tumors harvested at end of study were homogenized and processed for RNA and/or protein. Taqman assays were set up using reagents from Applied Biosystems (ABI, Foster City, Calif.) and assayed using 7500 Real Time PCR machine and software from ABI. Primer-probe sets were designed with primers flanking a fluorogenic probe dual labeled with Reporter dye FAM and quencher dye TAMRA. ETBR transcript levels in the tumors were normalized against transcript levels of reference genes such as Hprt1 (hypoxanthine phosphoribosyltransferase 1) or GAPDH (glyceraldehyde 3 phosphate dehydrogenase) using primer and probe sets that were specific to the human homologs of these genes.

The primer-probe set for reference gene Hprt1 (hypoxanthine phosphoribosyltransferase 1) is as follows:

Forward primer-5′
(SEQ ID NO: 17)
CAC ATC AAA GAC AGC ATC TAA GAA;
Reverse primer-5′
(SEQ ID NO: 18)
CAA GTT GGA AAA TAC AGT CAA CAT T
and
probe-5′
(SEQ ID NO: 19)
TTT TGT TCTGTC CTG GAA TTA TTT TAG TAG TGT TTC A.

The primer-probe set for ETBR is as follows:

Forward primer-5′
(SEQ ID NO: 14)
TCA CTG AAT TCC TGC ATT AAC C,
reverse primer-5′
(SEQ ID NO: 15)
GCA TAA GCA TGA CTT AAA GCA GTT
and
probe-5′
(SEQ ID NO: 16)
AAT TGC TCT GTATTT GGT GAG CAA AAG ATT CAA.

The primer-probe set for reference gene GAPDH (Glyceraldehyde 3 phosphate dehydrogenase) is as follows:

(SEQ ID NO: 20)
Forward primer-5′ GAA GAT GGT GAT GGG ATT TC,
(SEQ ID NO: 21)
Reverse primer-5′ GAA GGT GAA GGT CGG AGT C,
and
(SEQ ID NO: 22)
probe-5′ CAA GCT TCC CGT TCT CAG CC.

FIG. 27A shows that BRAFi induces ETBR mRNA in vivo as compared to control vehicle. FIG. 27B shows that MEKi-973 induces ETBR mRNA in vivo as compared to control vehicle as well.

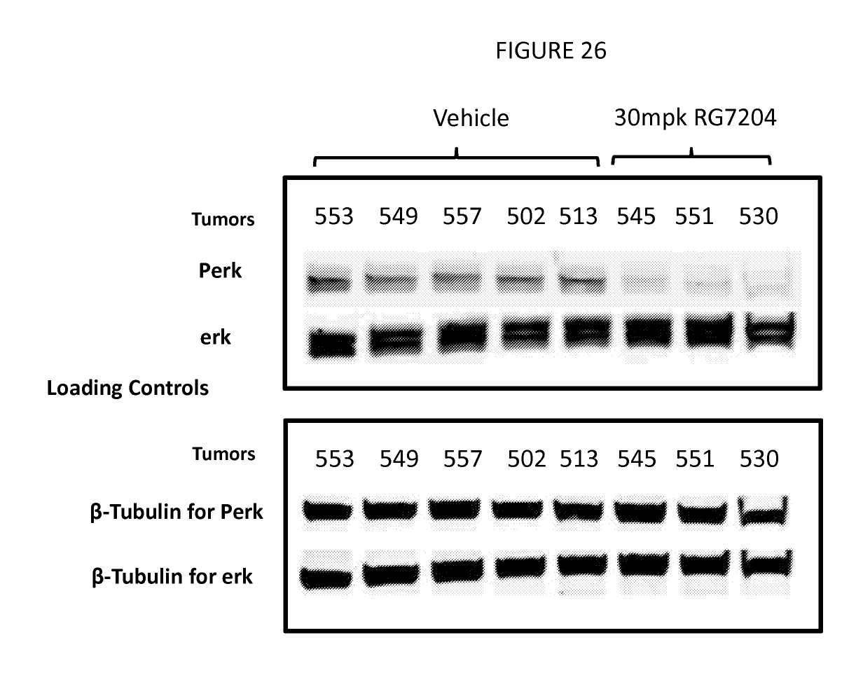

Phosphorylated erk and total erk protein levels were evaluated in the tumors by western blotting using the following reagents: for detection of proteins: anti-Phospho-p44/42 MAPK (Erk1/2) (Thr202/Tyr204) antibody (9101, Cell Signaling Technology), anti-p44/42 MAPK (Erk1/2) antibody (9102, Cell Signaling Technology) and mouse monoclonal anti-β-Tubulin antibody (556321, BD Pharmingen) as control (FIG. 26). Here, BRAFi appears to inhibit Perk in vivo as compared to control.

TABLE 3
PERCENT TUMOR GROWTH INHIBITION (TGI) SUMMARY

| | | | | % TGI | % TGI | | | |
|---|---|---|---|---|---|---|---|---|
| Melanoma | Anti-ETBR ADC | BRAFi | | Single Agent | Single Agent | % TGI | Delta from | |
| Xenograft | Single dose | Qdx21 | | Anti-ETBR ADC | BRAFi | Combination | Best Agent | |
| Model | Day 0 (mg/kg) | (mg/kg) | | vs. control | vs. control | vs. control | (Best Agent) | |
| UACC257-X2.2 | 1 | 1 | 945 | 69 | 66 | 127 | 58 | (ET_{B}R ADC) |
| UACC257-X2.2 | 1 | 6 | ↓ | 69 | 109 | 131 | 22 | 945 |
| UACC257-X2.2 | 3 | 1 | | 112 | 66 | 142 | 30 | (ET_{B}R ADC) |
| UACC257-X2.2 | 3 | 6 | | 112 | 109 | 148 | 36 | (ET_{B}R ADC) |
| UACC257-X2.2 | 3 | 20 | | 112 | 136 | 150 | 14 | 945 |
| COLO 829 | 1 | 10 | (RG7204) | 31 | 27 | 25 | −6 | (ET_{B}R ADC) |
| COLO 829 | 3 | 10 | ↓ | 33 | 27 | 71 | 38 | (ET_{B}R ADC) |
| COLO 829 | 1 | 30 | | 31 | 83 | 96 | 13 | (RG7204) |
| COLO 829 | 3 | 30 | | 33 | 83 | 105 | 22 | (RG7204) |
| A2058 | 3 | 10 | (RG7204) | 39 | 13 | 38 | −1 | (ET_{B}R ADC) |
| A2058 | 6 | 10 | ↓ | 85 | 13 | 102 | 17 | (ET_{B}R ADC) |
| A2058 | 3 | 30 | | 39 | 38 | 70 | 31 | (ET_{B}R ADC) |
| A2058 | 6 | 30 | | 85 | 38 | 100 | 15 | (ET_{B}R AC) |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of tumor growth inhibition (TGI) in a subject suffering from melanoma comprising administering to the subject an effective amount of an anti-endothelin B receptor (ETBR) antibody in combination with an effective amount of a MAP kinase inhibitor.

2. The method of claim 1, wherein said combination is synergistic.

3. The method of claim 1, wherein said TGI is greater than the TGI seen using an anti-ETBR antibody alone.

4. The method of claim 1, wherein said TGI is greater than the TGI seen using a MAP kinase inhibitor alone.

5. The method of claim 3, wherein the TGI is about 10% greater, or about 15% greater, or about 20% greater, or about 25% greater, or about 30% greater, or about 35% greater, or about 40% greater, or about 45% greater, or about 50% greater, or about 55% greater, or about 60% greater, or about 65% greater, or about 70% greater than use of an anti-ETBR antibody alone.

6. The method of claim 4, wherein the TGI is about 10% greater, or about 15% greater, or about 20% greater, or about 25% greater, or about 30% greater, or about 35% greater, or about 40% greater, or about 45% greater, or about 50% greater, or about 55% greater, or about 60% greater, or about 65% greater, or about 70% greater than use of a MAP kinase inhibitor alone.

7. The method of claim 1, wherein said anti-ETBR antibody specifically binds an ETBR epitope consisting of amino acids number 64 to 101 of SEQ ID NO:10.

8. The method of claim 1, wherein said anti-ETBR antibody has three variable heavy chain CDRs and three variable light chain CDRs wherein VH CDR1 is SEQ ID NO:1, VH CDR2 is SEQ ID NO:2, VH CDR3 is SEQ ID NO:3 and wherein VL CDR1 is SEQ ID NO:4, VL CDR2 is SEQ ID NO:5, VL CDR3 is SEQ ID NO:6.

9. The method of claim 1, wherein said anti-ETBR antibody has a variable heavy chain and a variable light chain, wherein said VH is SEQ ID NO:7 or 9.

10. The method of claim 9, wherein said VL is SEQ ID NO:8.

11. The method of claim 1, wherein said anti-ETBR antibody is conjugated to a cytotoxin.

12. The method of claim 11, wherein said cytotoxin is cytotoxic agent is selected from the group consisting of toxins, antibiotics, radioactive isotopes and nucleolytic enzymes.

13. The method of claim 12, wherein said cytotoxin is a toxin.

14. The method of claim 13, wherein said toxin is selected from the group consisting of maytansinoid, calicheamicin and auristatin.

15. (canceled)

16. The method of claim 1, wherein said MAP kinase inhibitor is a BRAF inhibitor.

17. The method of claim 1, wherein said BRAF inhibitor is propane-1-sulfonic acid {3-[5-(4-chlorophenyl)-1H-pyrrolo[2,3-b]pyridine-3-carbonyl]-2,4-difluoro-phenyl}-amide.

18. The method of claim 1, wherein said BRAF inhibitor has the following chemical structure:

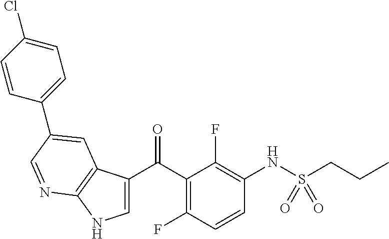 [SMILES: CCCS(=O)(=O)Nc1ccc(F)c(C(=O)c2c[nH]c3ncc(-c4ccc(Cl)cc4)cc23)c1F]

19. The method of claim 1, wherein said MAP kinase inhibitor is a MEK inhibitor.

20. The method of claim 1, wherein said MEK inhibitor is (S)-(3,4-difluoro-2-((2-fluoro-4-iodophenyl)amino)phenyl)(3-hydroxy-3-(piperidin-2yl)azetidin-1-yl)methanone.

21. The method of claim 1, wherein said MEK inhibitor has the following chemical structure:

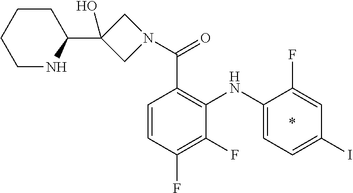 [SMILES: O=C(c1ccc(F)c(F)c1Nc1ccc(I)cc1F)N1CC(O)([C@@H]2CCCCN2)C1]

22. A method of treating melanoma comprising administering to a subject in need thereof a therapeutically effective amount of a MAP kinase inhibitor and an anti-ETBR antibody.

23-59. (canceled)

60. An article of manufacture for TGI in a subject suffering from melanoma comprising a package comprising an anti-ETBR antibody composition and a MAP kinase inhibitor composition.

61. An article of manufacture for treating melanoma in a subject comprising a package comprising an anti-ETBR antibody composition and a MAP kinase inhibitor composition.

62-77. (canceled)
